(19) United States
(12) Patent Application Publication
(10) Pub. No.: US 2020/0183279 A1
(43) Pub. Date: Jun. 11, 2020

(54) PHOTOSENSITIVE RESIN COMPOSITION, RESIST FILM, PATTERN FORMING METHOD, AND METHOD FOR MANUFACTURING ELECTRONIC DEVICE

(71) Applicant: FUJIFILM Corporation, Tokyo (JP)

(72) Inventors: Kazuhiro MARUMO, Haibara-gun (JP); Naohiro TANGO, Haibara-gun (JP); Ryo NISHIO, Haibara-gun (JP); Akira TAKADA, Haibara-gun (JP)

(73) Assignee: FUJI FILM Corporation, Tokyo (JP)

(21) Appl. No.: 16/789,634

(22) Filed: Feb. 13, 2020

Related U.S. Application Data
(63) Continuation of application No. PCT/JP2018/030050, filed on Aug. 10, 2018.

(30) Foreign Application Priority Data
Sep. 29, 2017 (JP) 2017-190835

(51) Int. Cl. G03F 7/039; G03F 7/004; C08L 33/14; C07D 277/66; C07D 277/68; C07D 263/57

(52) U.S. Cl. CPC G03F 7/0392; G03F 7/0045; C08L 33/14; G03F 7/0048; C07D 277/68; C07D 263/57; C07D 277/66

(57) ABSTRACT

A photosensitive resin composition includes a resin having a constitutional unit having an acid-decomposable group, a photoacid generator, a solvent, and a compound represented by Formula D. In Formula D, X^{D} represents an O atom or an S atom, R^{1D} represents a hydrogen atom, a hydrocarbon group, an acyl group, an acyloxy group, or an alkoxycarbonyl group, R^{2D} represents a substituent, nD represents an integer from 0 to 4, and two or more of R^{2D}'s may be bonded to each other to form a ring.

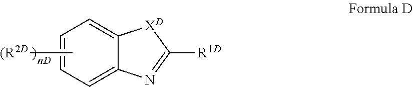

### CROSS-REFERENCE TO RELATED APPLICATIONS

This application is a Continuation of PCT International Application No. PCT/JP2018/030050 filed on Aug. 10, 2018, which claims priority under 35 U.S.C § 119(a) to Japanese Patent Application No. 2017-190835 filed on Sep. 29, 2017. Each of the above application(s) is hereby expressly incorporated by reference, in its entirety, into the present application.

### BACKGROUND OF THE INVENTION

### 1. Field of the Invention

The present disclosure relates to a photosensitive resin composition, a resist film, a pattern forming method, and a method for manufacturing an electronic device.

### 2. Description of the Related Art

In processes for manufacturing semiconductor devices such as an integrated circuit (IC) in the related art microfabrication by lithography using a photosensitive resin composition which is a so-called photoresist composition has been performed.

As such a photosensitive resin composition, for example, JP2014-002323A described a resist composition generating an acid upon exposure, whose solubility in a developer varies by the action of an acid, in which the resist composition contains a base component (A) whose solubility in a developer varies by the action of an acid, a nitrogen-containing compound (N) having a boiling point of 0° C. to 200° C. and a pKa of a conjugate acid of 0 to 7, and a photo base quenching agent (D1).

### SUMMARY OF THE INVENTION

In a case where exposure is performed using a photosensitive resin composition in the related art, it is required to improve a process margin in order to improve the yield.

In order to improve the process margin, a high tolerance of a depth of focus (DOF) considered to be important.

The present inventors have conducted extensive studies, and as a result, they have found that in a case of using the photosensitive resin composition described in JP2014-002323A, the tolerance of a depth of focus in a punched pattern such as a hole is not sufficient in some cases.

An object to be accomplished by embodiments of the present invention is to provide a photosensitive resin composition having a high tolerance of a depth of focus upon formation of a punched pattern, a resist film which is a solidified product of the photosensitive resin composition, a pattern forming method using the resist film, and a method for manufacturing an electronic device, using the resist film.

Examples of a means for accomplishing the object include the following aspects.

<1> A photosensitive resin composition comprising:

a resin having a constitutional unit having an acid-decomposable group;

a photoacid generator;

a solvent; and

a compound represented by Formula D,

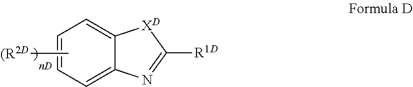

In Formula D, X^{D} represents an O atom or an S atom, R^{1D} represents a hydrogen atom, a hydrocarbon group, an acyl group, an acyloxy group, or an alkoxycarbonyl group, R^{2D} represents a halogen atom, an alkyl group, an alkenyl group, an aryl group, a hydroxyl group, an alkoxy group, an aryloxy group, a mercapto group, an alkylthio group, an arylthio group, an acyl group, an acyloxy group, an alkoxycarbonyl group, a cyano group, or a nitro group, nD represents an integer from 0 to 4, and two or more of R^{2D}'s may be bonded to each other to form a ring.

<2> The photosensitive resin composition as described in <1>,

in which a pKa of a conjugate acid of the compound represented by Formula D is 0.0 to 3.0.

<3> The photosensitive resin composition as described in <1> or <2>,

in which the compound represented by Formula D is a compound represented by Formula D-1.

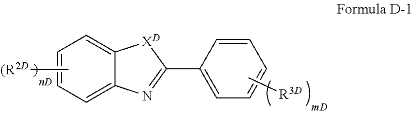

In Formula D-1, X^{D} represents an O atom or an S atom, R^{2D} and R^{3D} each independently represent a halogen atom, an alkyl group, an alkenyl group, an aryl group, a hydroxyl group, an alkoxy group, an aryloxy group, a mercapto group, an alkylthio group, an arylthio group, an acyl group, an acyloxy group, an alkoxycarbonyl group, a cyano group, or a nitro group, nD represents an integer from 0 to 4, mD represents an integer from 0 to 5, and two or more of R^{2D}'s may be bonded to each other to form a ring.

<4> The photosensitive resin composition as described in any one of <1> to <3>,

in which the compound represented by Formula D is a compound represented by Formula D-2.

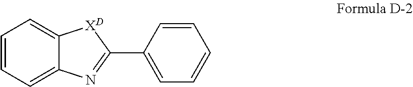

In Formula D-2, X^{D} represents an O atom or an S atom.

<5> The photosensitive resin composition as described in any one of <1> to <4>,

in which the photoacid generator is an ionic compound including a cation and an anion, and the anion includes an ion represented by any one of Formula An-1, Formula An-2, or Formula An-3.

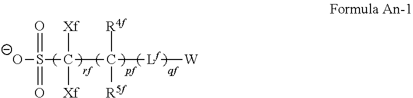

In Formula An-1, pf represents an integer of 0 to 10, qf represents an integer of 0 to 10, rf represents an integer of 1 to 3, Xf's each independently represent a fluorine atom or an alkyl group substituted with at least one fluorine atom, in a case where rf is an integer of 2 or more, a plurality of —C(Xf)₂-'s may be the same as or different from each other, R^{4f} and R^{5f} each independently represent a hydrogen atom, a fluorine atom, an alkyl group, or an alkyl group substituted with at least one fluorine atom, in a case where pf is an integer of 2 or more, a plurality of —CR^{4f}R^{5f}-'s may be the same as or different from each other, L^{f} represents a divalent linking group, in a case where qf is an integer of 2 or more, a plurality of L^{f}'s may be the same as or different from each other, and W represents an organic group including a cyclic structure.

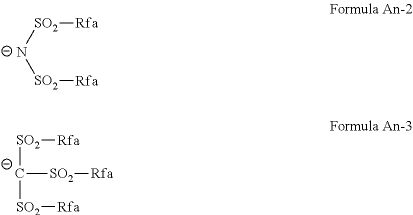

In Formula An-2 and Formula An-3, Rfa's each independently represent a monovalent organic group having a fluorine atom, and a plurality of Rfa's may be bonded to each other to form a ring.

<6> The photosensitive resin composition as described in any one of <1> to <5>,

in which the resin having a constitutional unit having an acid-decomposable group includes a constitutional unit represented by Formula III.

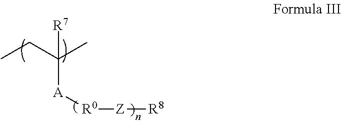

In Formula III. A represents an ester bond or an amide bond, n represents an integer of 0 to 5, R⁰ represents an alkylene group, a cycloalkylene group, or a combination thereof, Z represents a single bond, an ether bond, an ester bond, an amide bond, a urethane bond, or a urea bond, R⁸ represents a monovalent organic group having a lactone structure or a sultone structure, and R⁷ represents a hydrogen atom, a halogen atom, or a monovalent organic group.

<7> The photosensitive resin composition as described in any one of <1> to <6>,

in which the constitutional unit having an acid-decomposable group includes a constitutional unit represented by Formula AI.

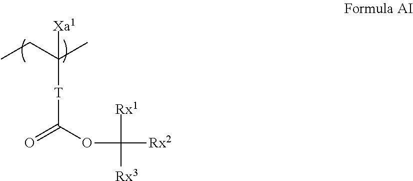

In Formula AI, Xa¹ represents a hydrogen atom, a halogen atom, or a monovalent organic group, T represents a single bond or a divalent linking group, Rx¹ to Rx³ each independently represent an alkyl group or a cycloalkyl group, and any two of Rx¹ to Rx³ may or may not be bonded to each other to form a ring structure.

<8> The photosensitive resin composition as described in any one of <1> to <7>, further comprising a fluorine-containing resin having a constitutional unit represented by Formula X.

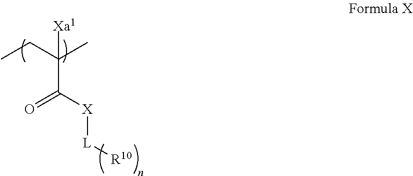

In Formula X, Z represents a halogen atom, a group represented by R¹¹OCH₂—, or a group represented by R¹² OC(═O)CH₂—, R¹¹ and R¹² each independently represent a substituent, X represents an oxygen atom or a sulfur atom, L represents an n+1-valent linking group, R¹⁰ represents a group having a group that decomposes by the action of an aqueous alkali solution to increase a solubility of the fluorine-containing resin in an aqueous alkali solution, n represents a positive integer, and in a case where n is 2 or more, a plurality of R¹⁰'s may be the same as or different from each other.

<9> The photosensitive resin composition as described in any one of <1> to <8>, further comprising a compound represented by Formula B.

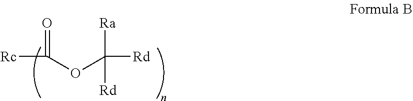

In Formula B, Ra represents an electron-withdrawing group, Rc represents an n-valent hydrocarbon group, Rd's each independently represent a hydrogen atom or a substituent, and n represents an integer of 1 to 3.

<10> The photosensitive resin composition as described in any one of <1> to <9>, further comprising a compound represented by any one of Formula d1-1, Formula d1-2, or Formula d1-3.

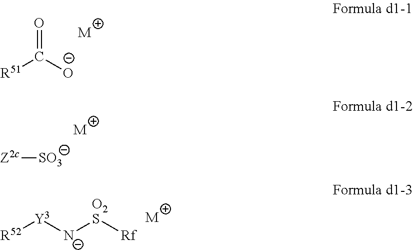

In Formula d1-1 to Formula d1-3, R⁵¹ represents a hydrocarbon group which may have a substituent, Z^{2c} represents a hydrocarbon group having 1 to 30 carbon atoms, which may have a substituent, in which a fluorine atom is not bonded to the carbon atom adjacent to the S atom, R⁵² represents an organic group, Y represents a linear, branched, or cyclic alkylene group or an arylene group, Rf represents a hydrocarbon group including a fluorine atom, and M¹'s each independently represent an ammonium cation, a sulfonium cation, or an iodonium cation.

<11> The photosensitive resin composition as described in any one of <1⁻>to <10>,

in which the solvent includes γ-butyrolactone.

<12> The photosensitive resin composition as described in any one of <1> to <11>, further comprising a low-molecular-weight ester compound having alkali decomposability and having a molecular weight of less than 1,500.

<13> A resist film which is a solidified product of the photosensitive resin composition as described in any one of <1> to <12>.

<14> A pattern forming method comprising:

exposing the resist film as described in <13>; and

developing the exposed resist film.

<15> A method for manufacturing an electronic device, comprising the pattern forming method as described in <14>.

According to the embodiments of the present invention, it is possible to provide a photosensitive resin composition having a high tolerance of a depth of focus upon formation of a punched pattern; a resist film which is a solidified product of the photosensitive resin composition; a pattern forming method using the resist film; and a method for manufacturing an electronic device, using the resist film.

### DESCRIPTION OF THE PREFERRED EMBODIMENTS

Hereinafter, the present invention will be described in detail.

Description of configuration requirements described below may be made on the basis of representative embodiments of the present invention in some cases, but the present invention is not limited to such embodiments.

In citations for a group (atomic group) in the present specification, in a case where the group is denoted without specifying whether it is substituted or unsubstituted, the group includes both a group not having a substituent and a group having a substituent. For example, an “alkyl group” includes not only an alkyl group not having a substituent (unsubstituted alkyl group), but also an alkyl group having a substituent (substituted alkyl group). In addition, an “organic group” in the present specification refers to a group including at least one carbon atom.

“Actinic rays” or “radiation” in the present specification means, for example, a bright line spectrum of a mercury lamp, far ultraviolet rays typified by an excimer laser, extreme ultraviolet rays (EUV rays), X-rays, electron beams (EB), or the like. “Light” in the present specification means actinic rays or radiation unless otherwise specified.

“Exposure” in the present specification encompasses, unless otherwise specified, not only exposure with a bright line spectrum of a mercury lamp, far ultraviolet rays typified by an excimer laser, extreme ultraviolet rays (EUV rays), X-rays, or the like but also exposure with particle rays such as electron beams and ion beams.

In the present specification, “(value) to (value)” is used to indicate a range that includes the preceding and succeeding numerical values of “to” as the lower limit value and the upper limit value, respectively.

In the present specification, (meth)acrylate represents acrylate and methacrylate, and (meth)acryl represents acryl and methacryl.

In the present specification, the weight-average molecular weight (Mw), the number-average molecular weight (Mn), and the dispersity (also referred to as a molecular weight distribution) (Mw/Mn) of a resin component are defined as values in terms of polystyrene by means of gel permeation chromatography (GPC) measurement (solvent: tetrahydrofuran, flow amount (amount of a sample injected): 10 μL, columns: TSK gel Multipore HXL-M manufactured by Tosoh Corporation, column temperature: 40° C., flow rate: 1.0 mL/min, and detector: differential refractive index detector) using a GPC apparatus (HLC-8120GPC manufactured by Tosoh Corporation).

In the present specification, in a case where a plurality of substances corresponding to each of components in a composition are present, the amount of each of components in the composition means the total amount of the plurality of the corresponding substances present in the composition unless otherwise specified.

In the present specification, the term “step” includes not only an independent step but also a step in which the anticipated effect of this step is achieved, even if the step cannot be clearly differentiated from the other steps.

In the present specification, the “total solid contents” refer to the total mass of components excluding a solvent from the total composition of a composition. Further, the “solid contents” are components excluding a solvent as described above, and may be, for example, either a solid or a liquid at 25° C.

In the present specification, “% by mass” and “% by weight” have the same definitions and “parts by mass” and “parts by weight” have the same definitions.

Furthermore, in the present specification, a combination of two or more of preferred aspects is a more preferred aspect.

(Photosensitive Resin Composition)

The photosensitive resin composition according to the present disclosure includes a resin having a constitutional unit having an acid-decomposable group, a photoacid generator, a solvent, and a compound represented by Formula D.

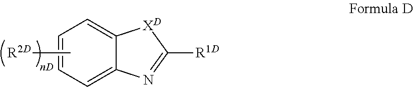

In Formula D, X^{D} represents an O atom or an S atom, R^{1D} represents a hydrogen atom, a hydrocarbon group, an acyl group, an acyloxy group, or an alkoxycarbonyl group, R^{2D} represents a halogen atom, an alkyl group, an alkenyl group, an aryl group, a hydroxyl group, an alkoxy group, an aryloxy group, a mercapto group, an alkylthio group, an arylthio group, an acyl group, an acyloxy group, an alkoxycarbonyl group, a cyano group, or a nitro group, nD represents an integer from 0 to 4, and two or more of R^{2D}'s may be bonded to each other to form a ring.

As described above, in a case where exposure is performed using a photosensitive resin composition in the related art, the tolerance of a depth of focus is required to be high in order to improve the yield.

In addition, the present inventors have found that in a case of using the photosensitive resin composition described in JP2014-002323A, the tolerance of a depth of focus in a punched pattern is not sufficient in some cases.

The present inventors have conducted extensive studies, and as a result, they have found that a photosensitive resin composition having a high tolerance of a depth of focus upon formation of a punched pattern is obtained by taking the configurations.

Detailed mechanism in which the effect is obtained is not clear, but it is presumed that since the compound represented by Formula D is a compound that has weaker basicity than an ordinary basic compound, it hardly inhibits an action of an acid generated from a photoacid generator in an exposed area, and sufficiently functions as a quencher (trapping agent) of an acid in the unexposed area. Further, it is presumed that since the compound represented by Formula D has appropriate diffusibility and can effectively control the diffusion of an acid generated, a photosensitive resin composition having a high tolerance of a depth of focus upon formation of a punched pattern can be obtained.

The photosensitive resin composition according to the present disclosure is preferably a resist composition, and may be either a positive-tone resist composition or a negative-tone resist composition. In addition, the composition may be either a resist composition for alkali development or a resist composition for organic solvent development.

The photosensitive resin composition according to the present disclosure is preferably a chemically amplified photosensitive resin composition.

Hereinafter, details of the respective components included in the photosensitive resin composition according to the present disclosure (also simply referred to as a “composition”) will be described.

<Compound Represented by Formula D>

The photosensitive resin composition according to the present disclosure includes the compound represented by Formula D.

In the compound represented by Formula D, it is presumed that a nitrogen atom in a benzoxazole ring structure or a benzothiazole ring structure acts as a weak base, and thus, a photosensitive resin composition having a high tolerance of a depth of focus upon formation of a punched pattern can be obtained as described above.

From the viewpoints of the tolerance of a depth of focus and the diffusion suppressing properties of the compound, X^{D} in Formula D is preferably an O atom, and from the viewpoints of the rectangularity of a pattern obtained and the tolerance of a depth of focus, X^{D} is preferably an S atom.

From the viewpoints of the tolerance of a depth of focus and the pattern linearity, R^{1D} in Formula D is preferably a hydrogen atom or a hydrocarbon group, more preferably the hydrogen atom, an alkyl group, or an aryl group, still more preferably the aryl group, and particularly preferably a phenyl group.

From the viewpoints of the tolerance of a depth of focus and the pattern linearity, the number of carbon atoms of R^{1D} in Formula D is preferably 0 to 20, more preferably 1 to 10, and still more preferably 4 to 8.

The hydrocarbon group, the acyl group, the acyloxy group, and the alkoxycarbonyl group in R^{1D} in Formula D may each have a substituent.

Examples of the substituent include a halogen atom, an alkyl group, an aryl group, a hydroxyl group, an alkoxy group, an aryloxy group, a mercapto group, an alkylthio group, an arylthio group, an acyl group, an acyloxy group, an alkoxycarbonyl group, a cyano group, and a nitro group.

In addition, the substituent may further be substituted by a substituent. Examples of the substituent include a halogenated alkyl group.

From the viewpoints of tolerance of a depth of focus and pattern linearity, R^{2D} in Formula D is preferably a halogen atom, an alkyl group, an alkenyl group, an aryl group, a hydroxyl group, an alkoxy group, or an aryloxy group, more preferably a fluorine atom, the alkyl group, the alkenyl group, the aryl group, the hydroxyl group, or the alkoxy group, and still more preferably the alkyl group. In a case where a plurality of R^{2D}'s are present, R^{2D}'s may be the same as or different from each other.

Furthermore, R^{2D} in Formula D may further have a substituent. Preferred examples of the substituent include the above-mentioned substituents.

In addition, a ring formed by the bonding of two or more of R^{2D}'s may be a saturated aliphatic ring, an unsaturated aliphatic ring, or an aromatic ring, but the ring is preferably the aromatic ring, and more preferably a benzene ring. That is, it is more preferably bonded to a benzene ring in the benzoxazole ring structure or the benzothiazole ring structure to form a naphthalene ring.

From the viewpoints of the tolerance of a depth of focus and the pattens linearity, nD in Formula D is preferably an integer from 0 to 3, more preferably 0 or 1, and particularly preferably 0.

From the viewpoints of the tolerance of a depth of focus and the pattern linearity, the pKa of a conjugate acid of the compound represented by Formula D is preferably −2.0 to 5.0, and more preferably 0.0 to 3.0.

In addition, the pKa in the present disclosure is a value in water and can be predictively calculated and determined using ACD/Labs ver 8.08 (manufactured by Fujitsu Ltd.).

From the viewpoints of the diffusion suppressing properties of the compound, the tolerance of a depth of focus and the pattern linearity, the boiling point of the compound represented by Formula D is preferably 200° C. or higher, more preferably 230° C. or higher, still more preferably 300° C. or higher, and particularly preferably from 350° C. to 450° C.

From the viewpoints of the tolerance of a depth of focus and the pattern linearity, it is preferable that the compound represented by Formula D is a compound represented by Formula D-1.

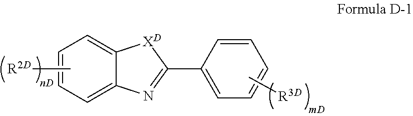

In Formula D-1, X^{D} represents an O atom or an S atom, R^{2D} and R^{3D} each independently represent a halogen atom, an alkyl group, an alkenyl group, an aryl group, a hydroxyl group, an alkoxy group, an aryloxy group, a mercapto group, an alkylthio group, an aiylthio group, an acyl group, an acyloxy group, an alkoxycarbonyl group, a cyano group, or a nitro group, nD represents an integer from 0 to 4, mD represents an integer from 0 to 5, and two or more of R^{2D}'s may be bonded to each other to form a ring.

X^{D}, and nD in Formula D-1 have the same definitions of X^{D}, R^{2D}, and nD, respectively, in Formula D, and preferred aspects thereof are each also the same.

From the viewpoints of the tolerance of a depth of focus and the pattern linearity, R^{3D}'s in Formula D-1 are each independently preferably a halogen atom, an alkyl group, an amyl group, a hydroxyl group, an alkoxy group, or an aryloxy group, and more preferably the alkyl group or the alkoxy group.

In addition, R^{3D} in Formula D-1 may further have a substituent. Preferred examples of the substituent include the above-mentioned substituents.

mD in Formula D-1 is preferably an integer from 0 to 3, more preferably 0 or 1, and particularly preferably 0.

From the viewpoints of the tolerance of a depth of focus and the pattern linearity, it is preferable that the compound represented by Formula D is a compound represented by Formula D-2.

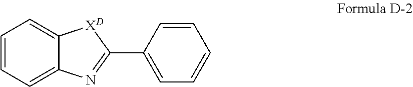

In Formula D-2, X^{D} represents an O atom or an S atom.

X^{D} in Formula D-2 has the same definition as X^{D} in Formula D and a preferred aspect thereof is also the same.

As specific preferred examples of the compound represented by Formula D, D-1 to D-13 are shown below, but the examples are not particularly limited thereto.

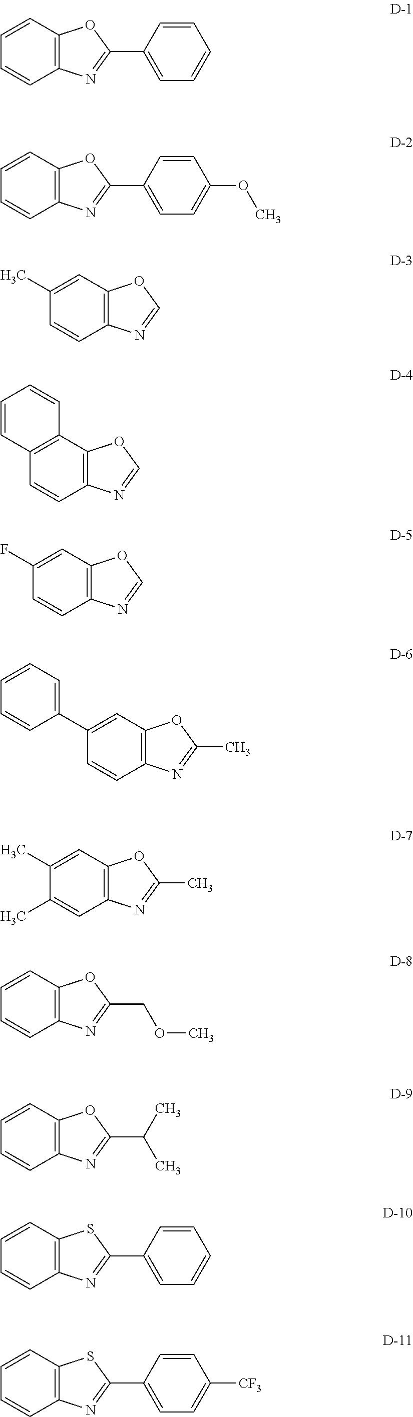

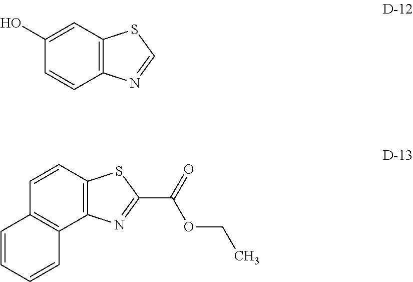 [SMILES: Oc1ccc2ncsc2c1 ; CCOC(=O)c1nc2c(ccc3ccccc32)s1]

Furthermore, the boiling points of D-1 to D-13 and the pKa of the conjugate acid are each values shown in Examples which will be described later.

In the photosensitive resin composition according to the present disclosure, the compounds represented by Formula D may be contained singly or may be used in combination of two or more kinds thereof.

From the viewpoints of the tolerance of a depth of focus and the pattern linearity, the content of the compound represented by Formula D is preferably from 0.1% by mass to 10% by mass, more preferably from 0.2% by mass to 5% by mass, and particularly preferably from 0.3% by mass to 2.5% by mass, with respect to the total solid content of the composition.

<Resin Having Constitutional Unit Having Acid-Decomposable Group>

The photosensitive resin composition according to the present disclosure preferably includes a resin having a constitutional unit having an acid-decomposable group (hereinafter also referred to as a “resin (A)”).

In this case, in a pattern forming method according to the present disclosure which will be described later, in the case where an alkali developer is employed as the developer, a positive-tone pattern is suitably formed, and in the case where an organic developer is employed as the developer, a negative-tone pattern is suitably formed.

[Constitutional Unit Having Acid-Decomposable Group]

The resin (A) has a constitutional unit having an acid-decomposable group.

As the resin (A), a known resin can be appropriately used. For example, the known resins disclosed in paragraphs 0055 to 0191 of US2016/0274458A, paragraphs 0035 to 0085 of US2015/0004544A, or paragraphs 0045 to 0090 of US2016/0147150A can be suitably used as the resin (A).

The acid-decomposable group preferably has a structure in which a polar group is protected with a group (leaving group) that leaves through decomposition by the action of an acid.

Examples of the polar group include an acidic group (a group that dissociates in a 2.38%-by-mass aqueous tetramethylammonium hydroxide solution) such as a carboxyl group, a phenolic hydroxyl group, a sulfonic acid group, a sulfonamido group, a sulfonylimido group, an (alkylsulfonyl)(alkylcarbonyl)methylene group, an (alkylsulfonyl)(alkylcarbonyl)imido group, a bis(alkylcarbonyl)methylene group, a bis(alkylcarbonyl)imido group, a bis(alkylsulfonyl)methylene group, a bis(alkylsulfonyl)imido group, a tris(alkylcarbonyl)methylene group, and a tris(alkylsulfonyl)methylene group, and an alcoholic hydroxyl group.

Moreover, the alcoholic hydroxyl group refers to a hydroxyl group bonded to a hydrocarbon group, which is a hydroxyl group other than a hydroxyl group (phenolic hydroxyl group) directly bonded to an aromatic ring, from which an aliphatic alcohol group (for example, a hexafluoroisopropanol group) having the a-position substituted with an electron-withdrawing group such as a fluorine atom is excluded as a hydroxyl group. The alcoholic hydroxyl group is preferably a hydroxyl group having an acid dissociation constant (pKa) from 12 to 20.

Preferred examples of the polar group include a carboxyl group, a phenolic hydroxyl group, and a sulfonic acid group.

A group which is preferable as the acid-decomposable group is a group in which a hydrogen atom of the leaving group is substituted with a group (leaving group) that leaves by the action of an acid.

Examples of the group (leaving group) that leaves by the action of an acid include —C(R³⁶)(R³⁷)(R³⁸), —C(R³⁶)(R³⁷)(OR³⁹), and —C(R⁰¹)(R⁰²)(OR³⁹).

In the formulae, R³⁶ to R³⁹ each independently represent an alkyl group, a cycloalkyl group, an aryl group, an aralkyl group, or an alkenyl group. R³⁶ and R³⁷ may be bonded to each other to form a ring.

R⁰¹ and R⁰² each independently represent a hydrogen atom, an alkyl group, a cycloalkyl group, an aryl group, an aralkyl group, or an alkenyl group,

As the alkyl group of each of R³⁶ to R³⁹, R⁰¹, and R⁰², an alkyl group having 1 to 8 carbon atoms is preferable, and examples thereof include a methyl group, an ethyl group, a propyl group, an n-butyl group, a sec-butyl group, a hexyl group, and an octyl group.

The cycloalkyl group of each of R³⁶ to R³⁹, R⁰¹, and R⁰² may be monocyclic or polycyclic. As the monocyclic cycloalkyl group, a cycloalkyl group having 3 to 8 carbon atoms is preferable, and examples thereof include a cyclopropyl group, a cyclobutyl group, a cyclopentyl group, a cyclohexyl group, and a cyclooctyl group. As the polycyclic cycloalkyl group, a cycloalkyl group having 6 to 20 carbon atoms is preferable, and examples thereof include an adamantyl group, a norbomyl group, an isobornyl group, a camphonyl group, a dicyclopentyl group, an α-pinel group, a tricyclodecanyl group, a tetracyclododecyl group, and an androstanyl group. Further, at least one carbon atom in the cycloalkyl group may be substituted with a heteroatom such as an oxygen atom.

The aryl group of each of R³⁶ to R³⁹, R⁰¹, and R⁰² is preferably an aryl group having 6 to 10 carbon atoms, and examples thereof include a phenyl group, a naphthyl group, and an anthiyl group.

The aralkyl group of each of R³⁶ to R³⁹, R⁰¹, and R⁰² is preferably an aralkyl group having 7 to 12 carbon atoms, and examples thereof include a benzyl group, a phenethyl group, and a naphthylmethyl group.

The alkenyl group of each of R³⁶ to R³⁹, R⁰¹, and R⁰² is preferably an alkenyl group having 2 to 8 carbon atoms, and examples thereof include a vinyl group, an allyl group, a butenyl group, and a cyclohexenyl group.

As the ring fanned by the bonding of R³⁶ and R³⁷, a (monocyclic or polycyclic) cycloalkyl group is preferable. As the cycloalkyl group, a monocyclic cycloalkyl group such as a cyclopentyl group and a cyclohexyl group, or a polycyclic cycloalkyl group such as a norbornyl group, a tetracyclodecanyl group, a tetracyclododecanyl group, and an adamantyl group is preferable.

The acid-decomposable group is preferably a cumyl ester group, an enol ester group, an acetal group, a tertiary alkyl ester group, or the like, and more preferably the acetal group or the tertiary alkyl ester group.

From the viewpoints of the tolerance of a depth of focus and the pattern linearity, it is preferable that the resin (A) has a constitutional unit represented by Formula AI as the constitutional unit having an acid-decomposable group.

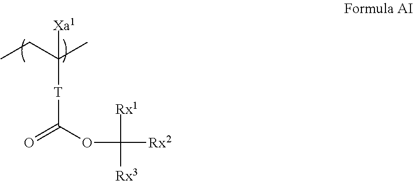

In Formula AI, Xa¹ represents a hydrogen atom, a halogen atom other than a fluorine atom, or a monovalent organic group, T represents a single bond or a divalent linking group, Rx¹ to Rx³ each independently represent an alkyl group or a cycloalkyl group, and any two of Rx¹ to Rx³ may or may not be bonded to each other to form a ring structure.

Examples of the divalent linking group of T include an alkylene group, an arylene group, —COO-Rt-, and —O-Rt-. In the formulae, Rt represents an alkylene group, a cycloalkylene group, or an arylene group, and

T is preferably the single bond or —COO-Rt-. Rt is preferably a chained alkylene group having 1 to 5 carbon atoms, and more preferably —CH₂—, —(CH₂)₂—, or —(CH₂)₃—. T is more preferably the single bond.

Xa¹ is preferably the hydrogen atom or the alkyl group.

The alkyl group of Xa¹ may have a substituent, and examples of the substituent include a hydroxyl group and a halogen atom other than a fluorine atom.

The alkyl group of Xa¹ preferably has 1 to 4 carbon atoms, and examples thereof include a methyl group, an ethyl group, a propyl group, and a hydroxymethyl group. The alkyl group of Xa¹ is preferably the methyl group.

The alkyl group of each of Rx¹, Rx², and Rx³ may be linear or branched, and preferred examples thereof include a methyl group, an ethyl group, an n-propyl group, an isopropyl group, an n-butyl group, an isobutyl group, and a t-butyl group. The number of the carbon atoms of the alkyl group is preferably 1 to 10, more preferably 1 to 5, and still more preferably 1 to 3. The alkyl group of each of Rx¹, Rx², and Rx³ may have some of carbon-carbon bonds that are double-bonded,

As the cycloalkyl group of each of Rx¹, Rx², and Rx³, a monocyclic cycloalkyl group such as a cyclopentyl group and a cyclohexyl group, or a polycyclic cycloalkyl group such as a norbornyl group, a tetracyclodecanyl group, a tetracyclododecanyl group, and an adamantyl group is preferable.

As the ring structure formed by the bonding of two of Rx¹, Rx², and Rx³, a monocyclic cycloalkane ring such as a cyclopentyl ring, a cyclohexyl ring, a cycloheptyl ring, and a cyclooctane ring, or a polycyclic cycloalkyl ring such as a norbornane ring, a tetracyclodecane ring, a tetracyclododecane ring, and an adamantane ring is preferable. The cyclopentyl ring, the cyclohexyl ring, or the adamantane ring is more preferable. As the ring structure formed by the bonding of two of Rx¹, Rx², and Rx³, a structure shown below is also preferable.

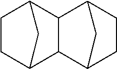 [SMILES: C1CC2CC1C1C3CCC(C3)C21]

Specific examples of a monomer corresponding to the constitutional unit represented by Formula AI are shown below, but the present disclosure is not limited to these specific examples. The following specific examples correspond to a case where Xa¹ in Formula AI is a methyl group, but Xa¹ can be optionally substituted with a hydrogen atom, a halogen atom other than a fluorine atom, or a monovalent organic group.

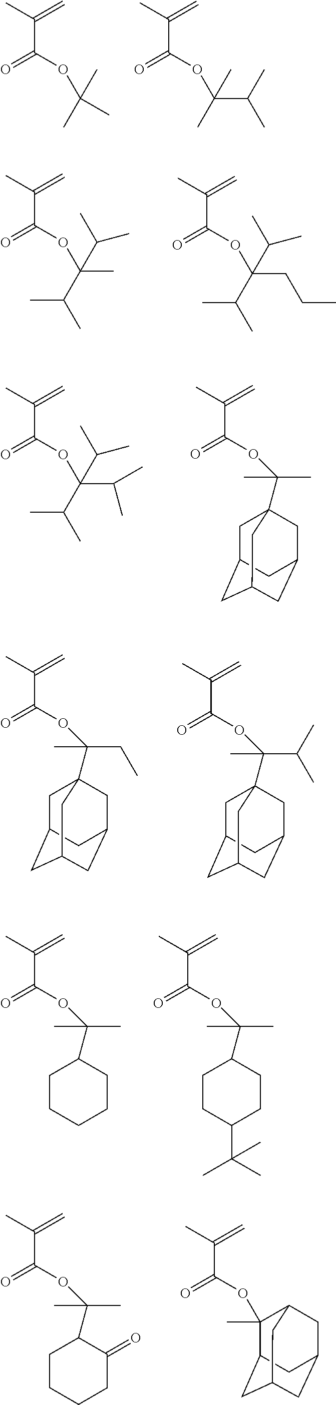

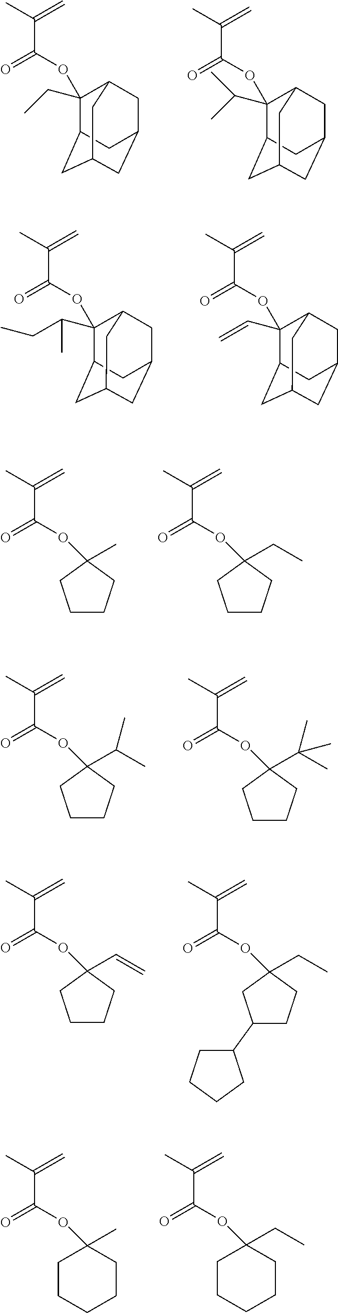

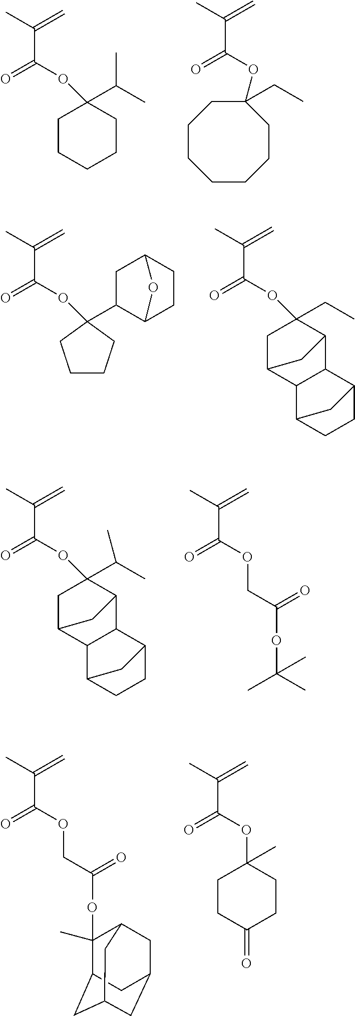

It is also preferable that the resin (A) has the constitutional unit described in paragraphs 0336 to 0369 of US2016/0070167A1 as the constitutional unit having an acid-decomposable group.

Moreover, the resin (A) may have a constitutional unit including a group that generates an alcoholic hydroxyl group through decomposition by the action of an acid, described in paragraphs 0363 and 0364 of US2016/0070167A1, as the constitutional unit having an acid-decomposable group.

The resin (A) may include only one kind or two or more kinds of the constitutional units having an acid-decomposable group.

The content of the constitutional unit having an acid-decomposable group (the total amount of the constitutional units having an acid-decomposable group in a case where a plurality of the constitutional units having an acid-decomposable group are present) included in the resin (A) is preferably 10% by mole to 90% by mole, more preferably 20% by mole to 80% by mole, and still more preferably 30% by mole to 70% by mole, with respect to all the constitutional units of the resin (A).

In addition, in a case where the content of the “constitutional unit” is defined in terms of a molar ratio in the present disclosure, the “constitutional unit” has the same definition as the “monomer unit”. Further, the “monomer unit” in the present disclosure may be modified after polymerization by a polymer reaction or the like. The same applies to the following.

[Constitutional Unit having at Least One Selected from Group Consisting of Lactone Structure, Sultone Structure, and Carbonate Structure]

The resin (A) preferably has a constitutional unit having at least one selected from the group consisting of a lactone structure, a sultone structure, and a carbonate structure.

As the lactone structure or the sultone structure, any structure is available as long as it has a lactone structure or a sultone structure, but the structure is preferably a 5- to 7-membered ring lactone structure or a 5- to 7-membered ring sultone structure, and more preferably a 5- to 7-membered ring lactone structure to which another ring structure is fused in the form of forming a bicyclo structure or a spiro structure or a 5- to 7-membered ring sultone structure to which another ring structure is fused in the form of forming a bicyclo structure or a spiro structure. The resin (A) still more preferably has a constitutional unit having a lactone structure represented by any one of Formulae LC1-1 to LC1-21 or a sultone structure represented by any one of Formulae SL1-1 to SL1-3. Further, the lactone structure or the sultone structure may be bonded directly to the main chain. Preferred examples of the structure include LC1-1, LC1-4, LC1-5, LC1-8, LC1-16, LC1-21, and SL1-1.

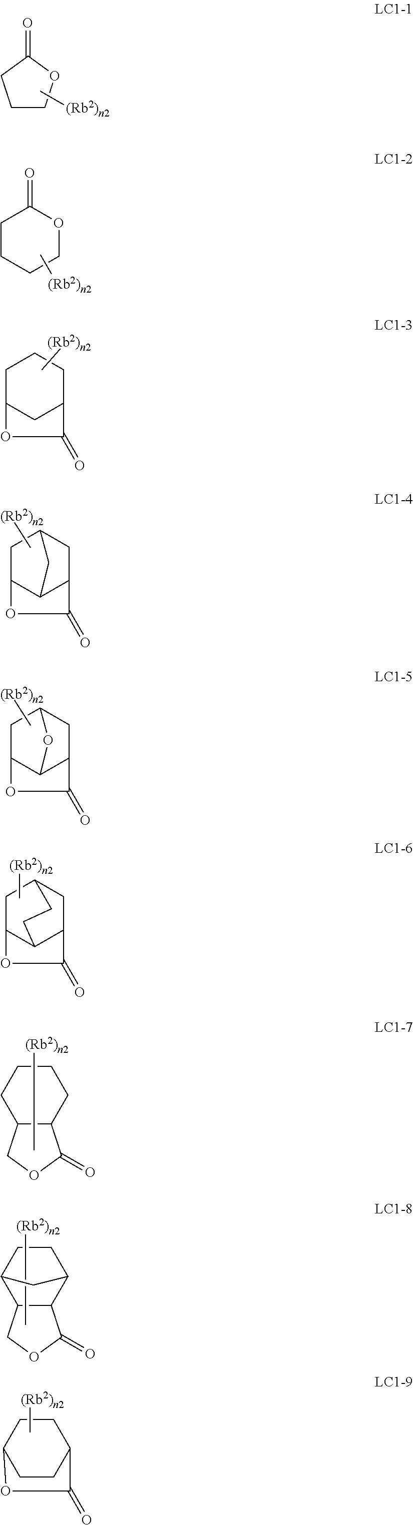

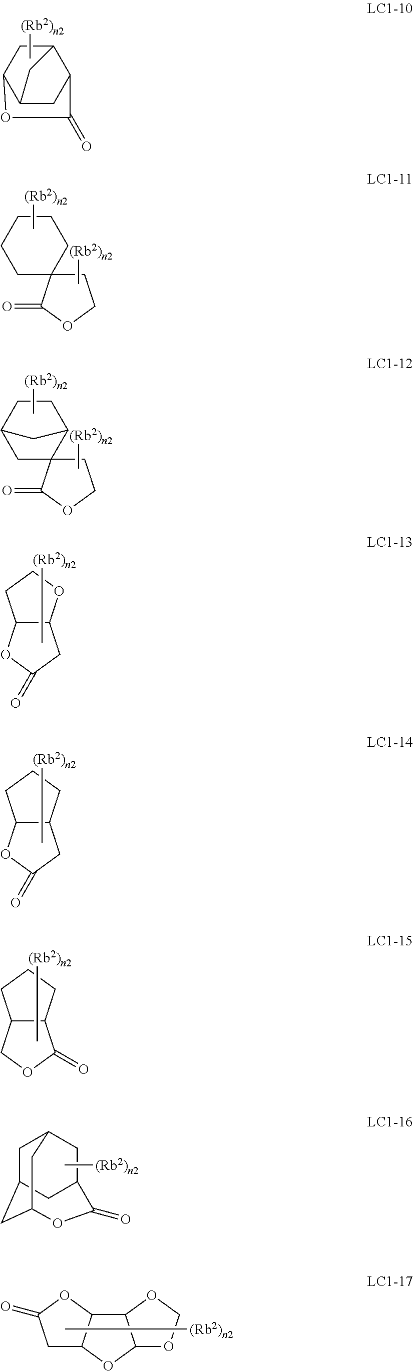

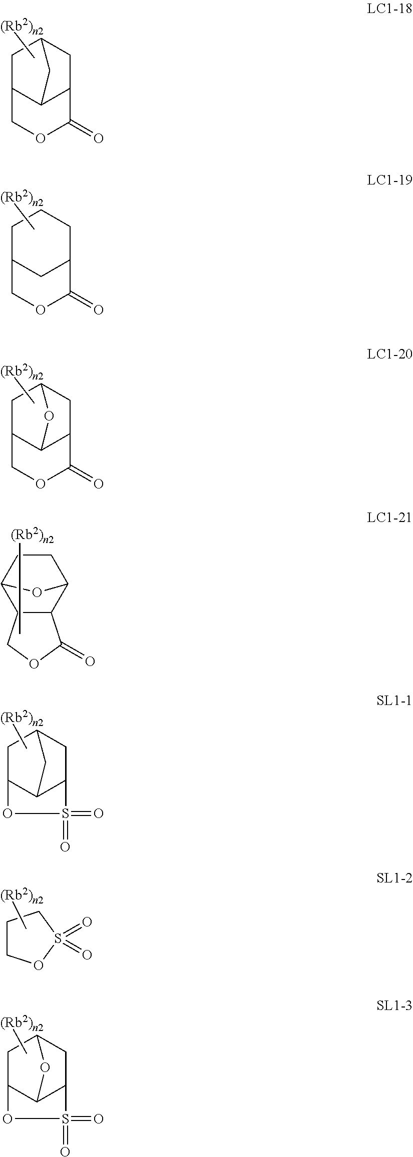

The lactone structural moiety or the sultone structural moiety may or may not have a substituent (Rb²). Preferred examples of the substituent (Rb²) include an alkyl group having 1 to 8 carbon atoms, a cycloalkyl group having 4 to 7 carbon atoms, an alkoxy group having 1 to 8 carbon atoms, an alkoxycarbonyl group having 2 to 8 carbon atoms, a carboxyl group, a halogen atom other than a fluorine atom, a hydroxyl group, a cyano group, and an acid-decomposable group is preferable. The substituent is more preferably the alkyl group having 1 to 4 carbon atoms, the cyano group, or the acid-decomposable group. n2 represents an integer of 0 to 4. In a case where n2 is 2 or more, the substituents (Rb²) which are present in plural number may be the same as or different from each other. Further, the substituents (Rb²) which are present in plural number may be bonded to each other to form a ring.

From the viewpoints of the tolerance of a depth of focus and the pattern linearity, it is preferable that the constitutional unit having a lactone structure or a sultone structure is a constitutional unit represented by Formula III.

In addition, from the viewpoints of the tolerance of a depth of focus and the pattern linearity, it is preferable that the resin having the constitutional unit having an acid-decomposable group includes the constitutional unit represented by Formula III.

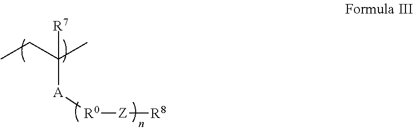

In Formula III,

A represents an ester bond (a group represented by —COO—) or an amide bond (a group represented by —CONH—).

n is the repetition number of the structure represented by —R⁰—Z—, represents an integer of 0 to 5, and is preferably 0 or 1, and more preferably 0. In a case where n is 0, —R⁰—Z— is not present, and A and R⁸ are bonded to each other through a single bond.

R⁰ represents an alkylene group, a cycloalkylene group, or a combination thereof. In a case where a plurality of R⁰'s are present, R⁰'s each independently represent an alkylene group, a cycloalkylene group, or a combination thereof.

Z represents a single bond, an ether bond, an ester bond, an amide bond, a urethane bond, or a urea bond. In a case where a plurality of Z's are present, Z's each independently represent a single bond, an ether bond, an ester bond, an amide bond, a urethane bond, or a urea bond.

R⁸ represents a monovalent organic group having a lactone structure or a sultone structure.

R⁷ represents a hydrogen atom, a halogen atom other than a fluorine atom, or a monovalent organic group (preferably a methyl group).

The alkylene group or the cycloalkylene group of R⁰ may have a substituent.

Z is preferably an ether bond or an ester bond, and more preferably the ester bond.

Specific examples of a monomer corresponding to the constitutional unit represented by Formula III and a monomer corresponding to the constitutional unit represented by Formula A-1 which will be described later are shown below, but the present disclosure is not particularly limited to these specific examples. The following specific examples correspond to a case where R⁷ in Formula III and R_{A}¹ in Formula A-1 which will be described later are each a methyl group, but R⁷ and R_{A}¹ can be optionally substituted with a hydrogen atom, a halogen atom other than a fluorine atom, or a monovalent organic group.

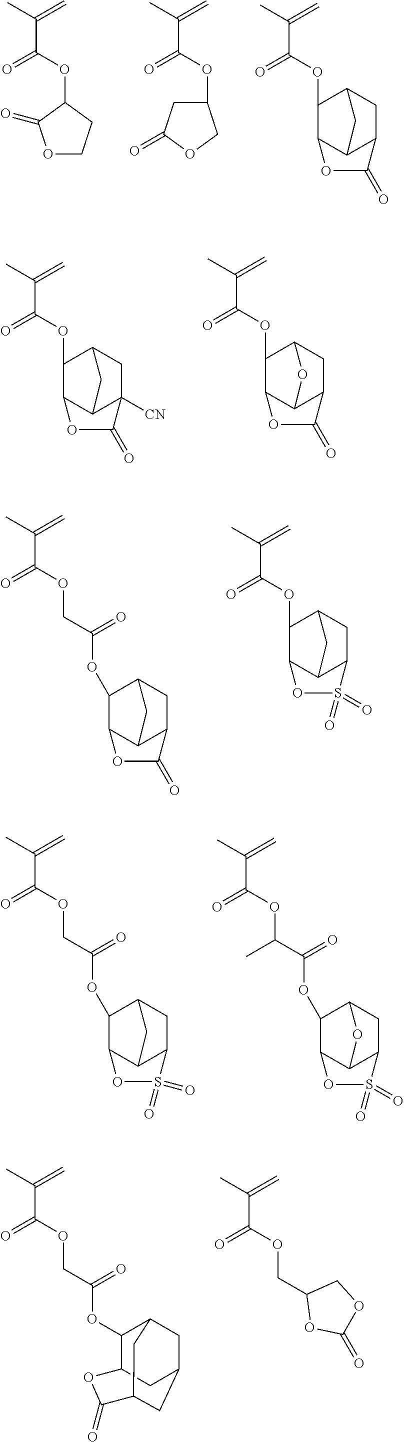

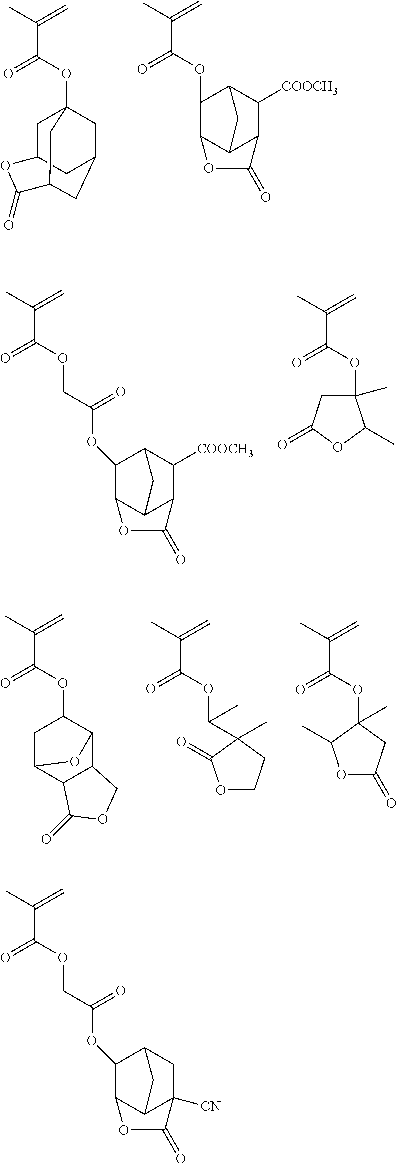

In addition to the monomers, monomers shown below are also suitably used as a raw material of the resin (A).

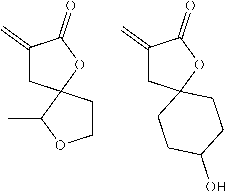

The resin (A) may have a constitutional unit having a carbonate structure. The carbonate structure is preferably a cyclic carbonic acid ester structure.

The constitutional unit having a cyclic carbonic acid ester structure is preferably a constitutional unit represented by Formula A-1.

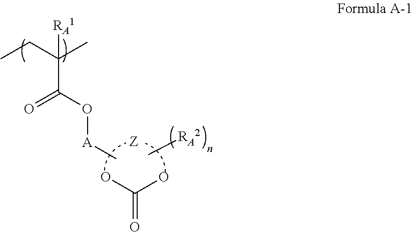

In Formula A-1, R_{A} represents a hydrogen atom, a halogen atom other than a fluorine atom, or a monovalent organic group (preferably a methyl group), n represents an integer of 0 or more, and R_{A}² represents a substituent. In a case where n is 2 or more, R_{A}²'s each independently represent a substituent, A represents a single bond or a divalent linking group, and Z represents an atomic group which forms a monocyclic structure or a polycyclic structure together with a group represented by —O—C(═O)—O— in the formula.

It is also preferable that the resin (A) has the constitutional unit described in paragraphs 0370 to 0414 of US2016/0070167A1 as the constitutional unit having at least one selected from the group consisting of a lactone structure, a sultone structure, and a carbonate structure.

The resin (A) may have only one kind or two or more kinds of the constitutional units having at least one selected from the group consisting of a lactone structure, a sultone structure, and a carbonate structure.

The content of the constitutional unit having at least one selected from the group consisting of a lactone structure, a sultone structure, and a carbonate structure (the total amount of the constitutional units having at least one selected from the group consisting of a lactone structure, a sultone structure, and a carbonate structure in a case where a plurality of the constitutional units having at least one selected from the group consisting of a lactone structure, a sultone structure, and a carbonate structure are present) included in the resin (A) is preferably 5% by mole to 70% by mole, more preferably 10% by mole to 65% by mole, and still more preferably 20% by mole to 60% by mole, with respect to all the constitutional units of the resin (A).

[Constitutional Unit Having Polar Group]

The resin (A) preferably has a constitutional unit having a polar group.

Examples of the polar group include a hydroxyl group, a cyano group, and a carboxyl group.

The constitutional unit having a polar group is preferably a constitutional unit having an alicyclic hydrocarbon structure substituted with a polar group. Further, it is preferable that the constitutional unit having a polar group has no acid-decomposable group. As the alicyclic hydrocarbon structure in the alicyclic hydrocarbon structure substituted with a polar group, an adamantyl group or a norbomyl group is preferable.

Specific examples of a monomer corresponding to the constitutional unit having a polar group are shown below, but the present disclosure is not particularly limited to these specific examples. Further, the following specific examples are described as a methacrylic acid ester compound, but they may be acrylic acid ester compounds.

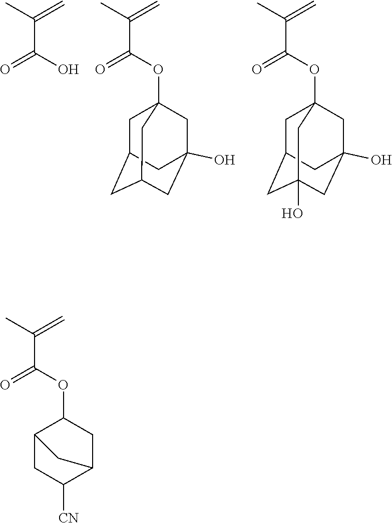

In addition, specific examples of the constitutional unit having a polar group include the constitutional units disclosed in paragraphs 0415 to 0433 of US2016/0070167A. The resin (A) may include only one kind or a combination of two or more kinds of the constitutional unit having a polar group.

The content of the constitutional unit having a polar group is preferably 5% by mole to 40% by mole, more preferably 5% by mole to 30% by mole, and still more preferably 10% by mole to 25% by mole, with respect to all the constitutional units in the resin (A).

[Constitutional Unit Having neither Acid-Decomposable Group nor Polar Group]

The resin (A) can further have a constitutional unit having neither an acid-decomposable group nor a polar group. The constitutional unit having neither an acid-decomposable group nor a polar group preferably has an alicyclic hydrocarbon structure. Examples of the constitutional unit having neither an acid-decomposable group nor a polar group include the constitutional units described in paragraphs 0236 and 0237 of US2016/0026083A. Preferred examples of a monomer corresponding to the constitutional unit having neither an acid-decomposable group nor a polar group are shown below.

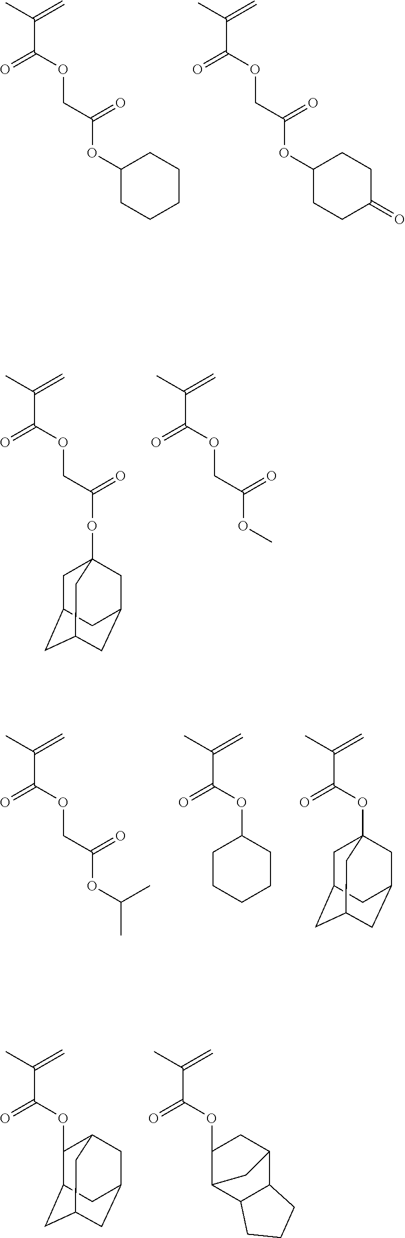

In addition, specific examples of the constitutional unit having neither an acid-decomposable group nor a polar group include the constitutional units disclosed in paragraph 0433 of US2016/0070167A.

The resin (A) may include only one kind or a combination of two or more kinds of the constitutional units having neither an acid-decomposable group nor a polar group.

The content of the constitutional unit having neither an acid-decomposable group nor a polar group is preferably 5% to 40% by mole, more preferably 5% to 30% by mole, and still more preferably 5% to 25% by mole, with respect to all the constitutional units in the resin (A).

The resin (A) may further have various constitutional units, in addition to the constitutional units, for the purpose of adjusting dry etching resistance, suitability for a standard developer, adhesiveness to a substrate, a resist profile, or resolving power, heat resistance, sensitivity, and the like which are general characteristics required for a resist. Examples of such a constitutional unit include constitutional units corresponding to the other monomers, but are not particularly limited thereto.

Examples of such the other monomers include a compound having one addition-polymerizable unsaturated bond, which is selected from acrylic acid esters, methacrylic acid esters, acrylamides, methacrylamides, allyl compounds, vinyl ethers, vinyl esters, and the like.

In addition to these compounds, an addition-polymerizable unsaturated compound that is copolymerizable with the monomers corresponding to various constitutional units as described above may be copolymerized.

In the resin (A), the molar ratio of each repeating constitutional unit contained is appropriately set in order to control various types of performance.

In a case where the photosensitive resin composition according to the present disclosure is for exposure with an argon fluoride (ArF) laser, from the viewpoint of transmittance of ArF light, it is preferable that the resin (A) does not substantially has an aromatic group. More specifically, the content of the constitutional unit having an aromatic group in all the constitutional units of the resin (A) is preferably 5% by mole or less, more preferably 3% by mole or less, and still more preferably ideally 0% by mole, that is, has no constitutional unit having an aromatic group. Further, the resin (A) preferably has a monocyclic or polycyclic alicyclic hydrocarbon structure.

It is preferable that all the constitutional units in the resin (A) are constituted with (meth)acrylate-based constitutional units. In this case, any of a resin in which all of the constitutional units are methacrylate-based constitutional units, a resin in which all of the constitutional units are acrylate-based constitutional units, and a resin in which all of the constitutional units are methacrylate-based constitutional units and acrylate-based constitutional units can be used, but it is preferable that the content of the acrylate-based constitutional units is 50% by mole or less with respect to all the constitutional units of the resin (A).

The weight-average molecular weight of the resin (A) is preferably 1,000 to 200,000, more preferably 2,000 to 20,000, still more preferably 3,000 to 15,000, and particularly preferably 3,000 to 11,000.

The dispersity (Mw/Mn) is preferably 1.0 to 3.0, more preferably 1.0 to 2.6, still more preferably 1.0 to 2.0, and particularly preferably 1.1 to 2.0.

Specific examples of the resin (A) include resins A-1 to A-15 used in Examples, but are not limited thereto.

The resin (A) may be used singly or in combination of two or more kinds thereof.

The content of the resin having a constitutional unit having an acid-decomposable group is preferably 20% by mass or more, more preferably 40% by mass or more, still more preferably 60% by mass or more, and particularly preferably 80% by mass or more, with respect to the total solid content of the photosensitive resin composition according to the present disclosure. An upper limit thereof is not particularly limited, but is preferably 99.5% by mass or less, more preferably 99% by mass or less, and still more preferably 97% by mass or less.

<Photoacid Generator>

The composition according to the present disclosure contains a photoacid generator (hereinafter also referred to as a “photoacid generator (C)”).

The photoacid generator is a compound that generates an acid upon irradiation with actinic rays or radiation.

As the photoacid generator, a compound that generates an organic acid upon irradiation with actinic rays or radiation is preferable. Examples thereof include a sulfonium salt compound, an iodonium salt compound, a diazonium salt compound, a phosphonium salt compound, an imide sulfonate compound, an oxime sulfonate compound, a diazodisulfone compound, a disulfone compound, and an o-nitrobenzyl sulfonate compound.

As the photoacid generator, known compounds that generate an acid upon irradiation with actinic rays or radiation can be appropriately selected and used singly or as a mixture thereof. For example, the known compounds disclosed in paragraphs 0125 to 0319 of US2016/0070167A1, paragraphs 0086 to 0094 of US2015/0004544A1, and paragraphs 0323 to 0402 of US2016/0237190A1 can be suitably used as the photoacid generator (C).

[Compounds Represented by Formulae ZI, ZII, and ZIII]

Suitable aspects of the photoacid generator (C) include, for example, compounds represented by Formulae ZI, ZII, and ZIII.

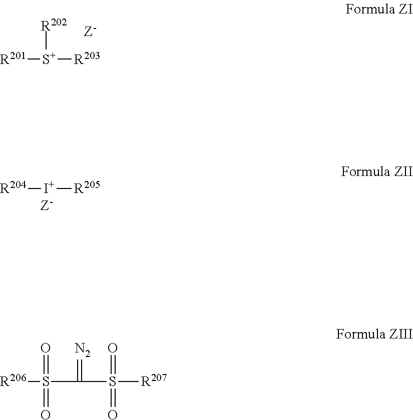

In Formula

R²⁰¹, R²⁰², and R²⁰³ each independently represent an organic group.

The number of carbon atoms of the organic group as each of R²⁰¹, R²⁰², and R²⁰³ is preferably 1 to 30, and more preferably 1 to 20.

In addition, two of R²⁰¹ to R²⁰³ may be bonded to each other to form a ring structure, and the ring may include an oxygen atom, a sulfur atom, an ester bond, an amide bond, or a carbonyl group. Examples of the group formed by the bonding of two of R²⁰¹ to R²⁰³ include an alkylene group (for example, a butylene group and a pentylene group) and —CH₂—CH₂—O—CH₂—CH₂—.

Z⁻ represents an anion.

[Cation in Compound Represented by Formula ZI]

Suitable aspects of the cation in Formula ZI include the corresponding groups in compounds (ZI-1), (ZI-2), (ZI-3), and (ZI-4) Which will be described later.

In addition, the photoacid generator (C) may be a compound having a plurality of the structures represented by Formula ZI. For example, it may be a compound having a structure in which at least one of R²⁰¹, . . . , or R²⁰³ in the compound represented by Formula ZI is bonded to at least one of R²⁰¹, . . . , or R²⁰³ of another compound represented by Formula ZI through a single bond or a linking group.

Compound ZI-1

First, the compound (ZI-1) will be described.

The compound (ZI-1) is an arylsulfonium compound in which at least one of R²⁰¹, . . . , or R²⁰³ in Formula ZI is an aryl group, that is, a compound having arylsulfonium as a cation.

In the arylsulfonium compound, all of R²⁰¹ to R²⁰³ may be aryl groups, or some of R²⁰¹ to R²⁰³ may be aryl groups and the remainders may be alkyl groups or cycloalkyl groups.

Examples of the arylsulfonium compound include a triarylsulfonium compound, a diarylalkylsulfonium compound, an aryldialkylsulfonium compound, a diarylcycloalkylsulfonium compound, and an aryldicycloalkylsulfonium compound.

As the aryl group included in the arylsulfonium compound, a phenyl group or a naphthyl group is preferable, and the phenyl group is more preferable. The aryl group may be an aryl group having a heterocyclic structure having an oxygen atom, a nitrogen atom, a sulfur atom, or the like. Examples of the heterocyclic structure include a pyrrole residue, a furan residue, a thiophene residue, an indole residue, a benzofuran residue, and a benzothiophene residue. In a case where the arylsulfonium compound has two or more aryl groups, these two or more aryl groups may be the same as or different from each other.

The alkyl group or the cycloalkyl group which may be contained, as necessary, in the arylsulfonium compound, is preferably a linear alkyl group having 1 to 15 carbon atoms, a branched alkyl group having 3 to 15 carbon atoms, or a cycloalkyl group having 3 to 15 carbon atoms, and examples thereof include a methyl group, an ethyl group, a propyl group, an n-butyl group, a sec-butyl group, a t-butyl group, a cyclopropyl group, a cyclobutyl group, and a cyclohexyl group.

The aryl group, the alkyl group, and the cycloalkyl group of each of R²⁰¹ to R²⁰³ may each independently have an alkyl group (for example, an alkyl group having 1 to 15 carbon atoms), a cycloalkyl group (for example, a cycloalkyl group having 3 to 15 carbon atoms), an aryl group (for example, an aryl group having 6 to 14 carbon atoms), an alkoxy group (for example, an alkoxy group having 1 to 15 carbon atoms), a halogen atom, a hydroxyl group, or a phenylthio group as a substituent.

Compound ZI-2

Next, the compound (ZI-2) will be described.

The compound (ZI-2) is a compound in which R²⁰¹ to R²⁰³ in Formula (ZI) each independently represent an organic group not having an aromatic ring. Here, the aromatic ring also encompasses an aromatic ring containing a heteroatom.

The organic group as each of R²⁰¹ to R²⁰³, which contains no aromatic ring, preferably has 1 to 30 carbon atoms, and more preferably has 1 to 20 carbon atoms.

R²⁰¹ to R²⁰³ are each independently preferably an alkyl group, a cycloalkyl group, an allyl group, or a vinyl group, more preferably a linear or branched 2-oxoalkyl group, a 2-oxocycloalkyl group, or an alkoxycarbonylmethyl group, and still more preferably the linear or branched 2-oxoalkyl group.

Preferred examples of the alkyl group and the cycloalkyl group of each of R²⁰¹ to R²⁰³ include a linear alkyl group having 1 to 10 carbon atoms or a branched alkyl group having 3 to 10 carbon atoms (for example, a methyl group, an ethyl group, a propyl group, a butyl group, and a pentyl group), and a cycloalkyl group having 3 to 10 carbon atoms (for example, a cyclopentyl group, a cyclohexyl group, and a norbornyl group).

R²⁰¹ to R²⁰³ may further be substituted with a halogen atom, an alkoxy group (for example, an alkoxy group having 1 to 5 carbon atoms), a hydroxyl group, a cyano group, or a nitro group.

Compound ZI-3

Next, the compound (ZI-3) will be described.

The compound (ZI-3) is a compound which is represented by Formula ZI-3 and has a phenacylsulfonium salt structure.

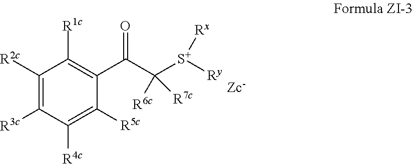

In Formula ZI-3A, R^{1c} to R^{5c} each independently represent a hydrogen atom, an alkyl group, a cycloalkyl group, an aryl group, an alkoxy group, an aryloxy group, an alkoxycarbonyl group, an alkylcarbonyloxy group, a cycloalkylcarbonyloxy group, a halogen atom, a hydroxyl group, a nitro group, an alkylthio group, or an arylthio group, R^{6c} and R^{7c} each independently represents a hydrogen atom, an alkyl group, a cycloalkyl group, a halogen atom, a cyano group, or an aryl group, and R^{x} and R^{y} each independently represent an alkyl group, a cycloalkyl group, a 2-oxoalkyl group, a 2-oxocycloalkyl group, an alkoxycarbonylalkyl group, an allyl group, or a vinyl group.

Each of any two or more of R^{1c}, . . . , or R^{5c}, and R⁵ and R^{6c}, R^{6c} and R^{7c}, R^{5c} and R^{x}, and R^{x} and R^{y} may be bonded to each other to form a ring structure, and this ring structure may each independently include an oxygen atom, a sulfur atom, a ketone group, an ester bond, or an amide bond.

Examples of the ring structure include an aromatic or non-aromatic hydrocarbon ring, an aromatic or non-aromatic heterocycle, or a polycyclic fused ring composed of two or more of these rings. Examples of the ring structure include 3- to 10-membered rings, and the ring structures are preferably 4- to 8-membered ring, and more preferably 5- or 6-membered rings.

Examples of the group formed by the bonding of each of any two or more of R^{1c}, . . . , or R^{5c}, R^{6c} and R^{7c}, and R^{x} and R^{y} include a hutylene group and a pentylene group.

As groups formed by the bonding of R^{5c} and R^{6c}, and R^{5c} and R^{x}, a single bond or an alkylene group is preferable. Examples of the alkylene group include a methylene group and an ethylene group.

Zc⁻ represents an anion.

Compound ZI-4

Next, the compound (ZI-4) will be described.

The compound (ZI-4) is represented by Formula ZI-4.

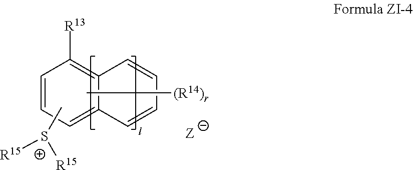

In Formula ZI-4, 1 represents an integer of 0 to 2, r represents an integer of 0 to 8, R¹³ represents a hydrogen atom, a fluorine atom, a hydroxyl group, an alkyl group, a cycloalkyl group, an alkoxy group, an alkoxycarbonyl group, or a group having a cycloalkyl group, each of which may have a substituent, R¹⁴'s each independently represent a hydroxyl group, an alkyl group, a cycloalkyl group, an alkoxy group, an alkoxycarbonyl group, an alkylcarbonyl group, an alkylsulfonyl group, a cycloalkylsulfonyl group, or a group having a cycloalkyl group, each of which may have a substituent, R¹⁵'s each independently represent an alkyl group, a cycloalkyl group, or a naphthyl group, each of which may have a substituent, and two R¹⁵'s may be bonded to each other to form a ring.

In a case where two R¹⁵'s are bonded to each other to form a ring, the ring skeleton may include a heteroatom such as an oxygen atom and a nitrogen atom. In one aspect, it is preferable that two R¹⁵'s are alkylene groups, and are bonded to each other to form a ring structure.

Z⁻ represents an anion.

In Formula ZI-4, the alkyl group of each of R¹³, R¹⁴, and R¹⁵ is linear or branched, the number of carbon atoms of the alkyl group is preferably 1 to 10, and the alkyl group is more preferably a methyl group, an ethyl group, an n-butyl group, a t-butyl group, or the like.

[Cation in Compound Represented by Formula ZII or Formula ZIII]

Next, Formulae ZII and ZIII will be described.

In Formulae ZII and ZIII, R²⁰⁴ to R²⁰⁷ each independently represent an aryl group, an alkyl group, or a cycloalkyl group.

As the amyl group of each of R²⁰⁴ to R²⁰⁷, a phenyl group or a naphthyl group is preferable, and the phenyl group is more preferable. The aryl group of each of R²⁰⁴ to R²⁰⁷ may be an aryl group having a heterocyclic structure having an oxygen atom, a nitrogen atom, a sulfur atom, or the like. Examples of the skeleton of the aryl group having a heterocyclic structure include pyrrole, furan, thiophene, indole, benzofuran, and benzothiophene.

Preferred examples of the alkyl group and the cycloalkyl group of each of R²⁰⁴ to R²⁰⁷ include a linear alkyl group having 1 to 10 carbon atoms or a branched alkyl group having 3 to 10 carbon atoms (for example, a methyl group, an ethyl group, a propyl group, a butyl group, and a pentyl group), and a cycloalkyl group having 3 to 10 carbon atoms (a cyclopentyl group, a cyclohexyl group, and a norbornyl group).

The aryl group, the alkyl group, and the cycloalkyl group of each of R²⁰⁴ to R²⁰⁷ may each independently have a substituent. Examples of the substituent which may be contained in the aryl group, the alkyl group, or the cycloalkyl group of each of R²⁰⁴ to R²⁰⁷ include an alkyl group (for example, having 1 to 15 carbon atoms), a cycloalkyl group (for example, having 3 to 15 carbon atoms), an aryl group (for example, having 6 to 15 carbon atoms), an alkoxy group (for example, having 1 to 15 carbon atoms), a halogen atom, a hydroxyl group, and a phenylthio group.

Z⁻ represents an anion.

[Anions in Compounds Represented by Formula ZI to Formula ZIII]

As Z⁻ in Formula ZI, Z⁻in Formula ZII, Zc⁻ in Formula ZI-3, and Z⁻ in Formula ZI-4, an anion represented by Formula An-1 is preferable.

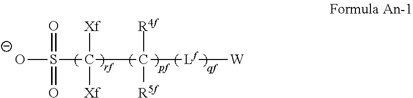

In Formula An-1, pf represents an integer of 0 to 10, of represents an integer of 0 to 10, if represents an integer of 1 to 3, Xf's each independently represent a fluorine atom or an alkyl group substituted with at least one fluorine atom, in a case where rf is an integer of 2 or more, a plurality of —C(Xf)₂-'s may be the same as or different from each other, R^{4f} and R^{5f} each independently represent a hydrogen atom, a fluorine atom, an alkyl group, or an alkyl group substituted with at least one fluorine atom, in a case where pf is an integer of 2 or more, a plurality of —CR^{4f}R^{5f}-'s may be the same as or different from each other, L^{f} represents a divalent linking group, in a case where of is an integer of 2 or more, a plurality of L^{f}'s may be the same as or different from each other, and W represents an organic group including a cyclic structure.

Xf represents a fluorine atom or an alkyl group substituted with at least one fluorine atom. The number of carbon atoms of the alkyl group is preferably 1 to 10, and more preferably 1 to 4. Further, the alkyl group substituted with at least one fluorine atom is preferably a perfluoroalkyl group.

Xf is preferably the fluorine atom or the perfluoroalkyl group having 1 to 4 carbon atoms. Xf is more preferably the fluorine atom or CF₃. It is particularly preferable that both Xf's are fluorine atoms.

R^{4f} and R^{5f} each independently represent a hydrogen atom, a fluorine atom, an alkyl group, or an alkyl group substituted with at least one fluorine atom. In a case where a plurality of each of R^{4f}'s and R^{5f}'s are present, R^{4f}'s and R^{5f}'s may be the same as or different from each other.

The alkyl group represented by each of R^{4f} and R^{5f} may have a substituent, and preferably has 1 to 4 carbon atoms. R^{4f} and R^{5f} are each preferably a hydrogen atom.

Specific examples and suitable aspects of the alkyl group substituted with at least one fluorine atom are the same as the specific examples and the suitable aspects of Xf in Formula An-1.

L^{f} represents a divalent linking group, and in a case where a plurality of L^{f}'s are present, L^{f}'s may be the same as or different from each other.

Examples of the divalent linking group include —COO—(—C(═O)—O—), —OCO—, —CONH—, —NHCO—, —CO—, —O—, —S—, —SO—, —SO₂—, an alkylene group (preferably having 1 to 6 carbon atoms), a cycloalkylene group (preferably having 3 to 15 carbon atoms), an alkenylene group (preferably having 2 to 6 carbon atoms), or a divalent linking group formed by combination of these plurality of groups. Among these, —COO—, —OCO—, —CONH—, —NHCO—, —CO—, —O—, —SO₂—, —COO-alkylene group-, —OCO-alkylene group-, —CONH-alkylene group-, or —NHCO-alkylene group- is preferable, and —COO—, —OCO—, —CONH—, —SO₂—, —COO-alkylene group-, or —OCO-alkylene group- is more preferable.

W represents an organic group including a cyclic structure. Among these, W is preferably a cyclic organic group.

Examples of the cyclic organic group include an alicyclic group, an aryl group, and a heterocyclic group.

The alicyclic group may be monocyclic or polycyclic. Examples of the monocyclic alicyclic group include monocyclic cycloalkvl groups such as a cyclopentyl group, a cyclohexyl group, and a cyclooctyl group. Examples of the polycyclic alicyclic group include polycyclic cycloalkyl groups such as a norbornyl group, a tricyclodecanyl group, a tetracyclodecanyl group, a tetracyclododecanyl group, and an adamantyl group. Among those, an alicyclic group having a bulky structure having 7 or more carbon atoms, such as a norbornyl group, a tricyclodecanyl group, a tetracyclodecanyl group, a tetracyclododecanyl group, and an adamantyl group is preferable.

The aryl group may be monocyclic or polycyclic. Examples of the aryl group include a phenyl group, a naphthyl group, a phenanthryl group, and an anthrvl group.

The heterocyclic group may be monocyclic or polycyclic. In a case where it is polycyclic, it is possible to suppress acid diffusion. Further, the heterocyclic group may have aromaticity or may not have aromaticity. Examples of the heterocycle having aromaticity include a furan ring, a thiophene ring, a benzofuran ring, a benzothiophene ring, a dibenzofuran ring, a dibenzothiophene ring, and a pyridine ring. Examples of the heterocycle not having aromaticity include a tetrahydropyran ring, a lactone ring, a sultone ring, and a decahydroisoquinoline ring. Examples of the lactone ring and the sultone ring include the above-mentioned lactone structures and sultone structures exemplified in the resin. As the heterocycle in the heterocyclic group, a furan ring, a thiophene ring, a pyridine ring, or a decahydroisoquinoline ring is particularly preferable.

The cyclic organic group may have a substituent. Examples of the substituent include, an alkyl group (which may be linear or branched, and preferably has 1 to 12 carbon atoms), a cycloalkyl group (which may be any one of a monocycle, a polycycle, and a spiro ring, and preferably has 3 to 20 carbon atoms), an aryl group (preferably having 6 to 14 carbon atoms), a hydroxyl group, an alkoxy group, an ester group, an atnido group, a urethane group, a ureido group, a thioether group, a sulfonamido group, and a sulfonic acid ester group. Incidentally, the carbon constituting the cyclic organic group (carbon contributing to ring formation) may be carbonyl carbon.

Preferred examples of the anion represented by Formula An-1 include SO₃⁻—CF₂—CH₂—OCO-(L^{f})q′-W, SO₃⁻—CF₂—CHF—CH₂—OCO-(L^{f})q′-W, SO₃⁻-CF₂—COO-(L^{f})q′-W, SO₃⁻—CF₂-CF₂-CH₂-CH₂-(L^{f})_{qf}-W, and SO₃⁻-CF₂—CH(CF₃)—OCO-(L^{f})q′-W. Here, L^{f}, qf, and W are each the same as those in Formula An-1. q′ represents an integer of 0 to 10.

In one aspect, as Z⁻ in Formula ZI, Z⁻ in Formula ZII, Zc⁻ in Formula ZI-3, and Z⁻ in Formula ZI-4, an anion represented by Formula 4 is also preferable.

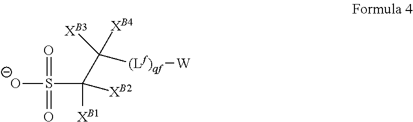

In Formula 4, X^{B1} and X^{B2} each independently represent a hydrogen atom or a monovalent organic group having no fluorine atom. X^{B1} and X^{B2} are each preferably a hydrogen atom.

X^{B3} and X^{B4} each independently represent a hydrogen atom or a monovalent organic group. It is preferable that at least one of X^{B3} or X^{B4} is a fluorine atom or a monovalent organic group having a fluorine atom, and it is more preferable that both of X^{B3} and X^{B4} are a fluorine atom or a monovalent organic group having a fluorine atom. It is still more preferable that X^{B3} and X^{B4} are both an alkyl group substituted with fluorine.

L^{f}, qf, and W are the same as those in Formula 3.

As Z⁻ in Formula ZI, Z⁻in Formula ZII, Zc⁻ in Formula ZI-3, and Z⁻ in Formula ZI-4, an anion represented by Formula 5 is preferable.

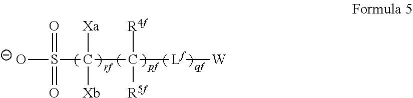

In Formula 5, Xa's each independently a fluorine atom or an alkyl group substituted with at least one fluorine atom, and Xb's each independently a hydrogen atom or an organic group having no fluorine atom. The definitions and preferred aspects of rf, pf, qf, R^{4f}, R^{5f}, L^{f}, and W are each the same as those in Formula 3.

Z⁻ in Formula ZI, Z⁻ in Formula ZII, Ze⁻ in Formula ZI-3, and Z⁻ in Formula ZI-4 may be each a benzenesulfonate anion, and are each preferably a benzenesulfonate anion substituted with a branched alkyl group or a cycloalkyl group.

As Z⁻ in Formula ZI, Z⁻ in Formula ZII, Zc⁻ in Formula ZI-3, and Z⁻ in Formula. ZI-4, an aromatic sulfonate anion represented by Formula SA1 is also preferable.

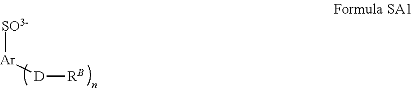

In Formula SA1, Ar represents an aryl group and may further have a substituent other than a sulfonate anion and -(D-R^{B}). Examples of the substituent that may further be contained include a fluorine atom and a hydroxyl group.

n represents an integer of 0 or more n is preferably 1 to 4, more preferably 2 or 3, and particularly preferably 3.

D represents a single bond or a divalent linking group. Examples of the divalent linking group include an ether group, a thioether group, a carbonyl group, a sulfoxide group, a sulfone group, a sulfonic acid ester group, an ester group, and a group formed by combination of two or more kinds of these groups.

R^{B} represents a hydrocarbon group.

It is preferable that D is the single bond and R^{B} is an aliphatic hydrocarbon structure, It is more preferable that R^{B} is an isopropyl group or a cyclohexyl group.

Preferred examples of the sulfonium cation in Formula ZI and the sulfonium cation or iodonium cation in Formula ZII are shown below.

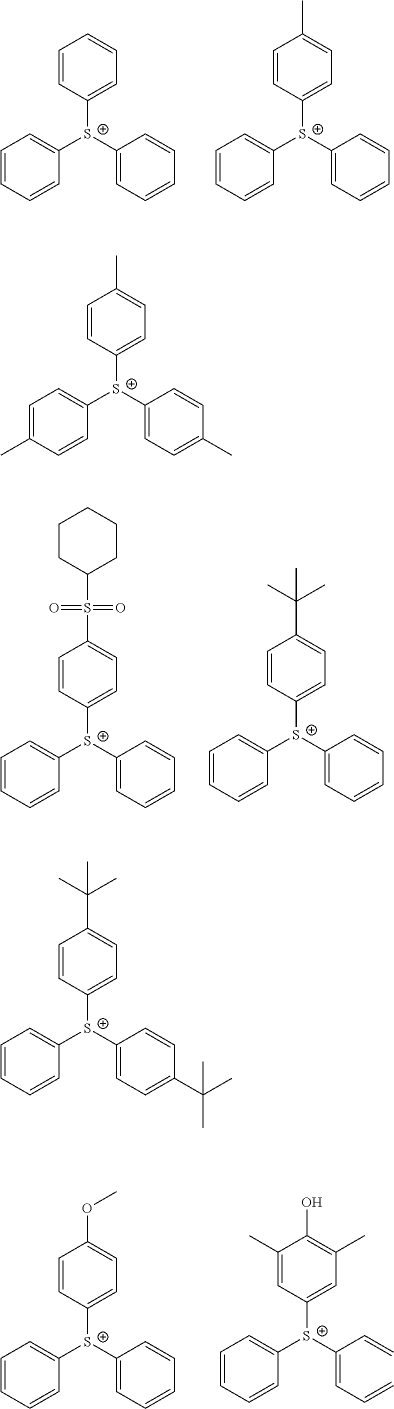

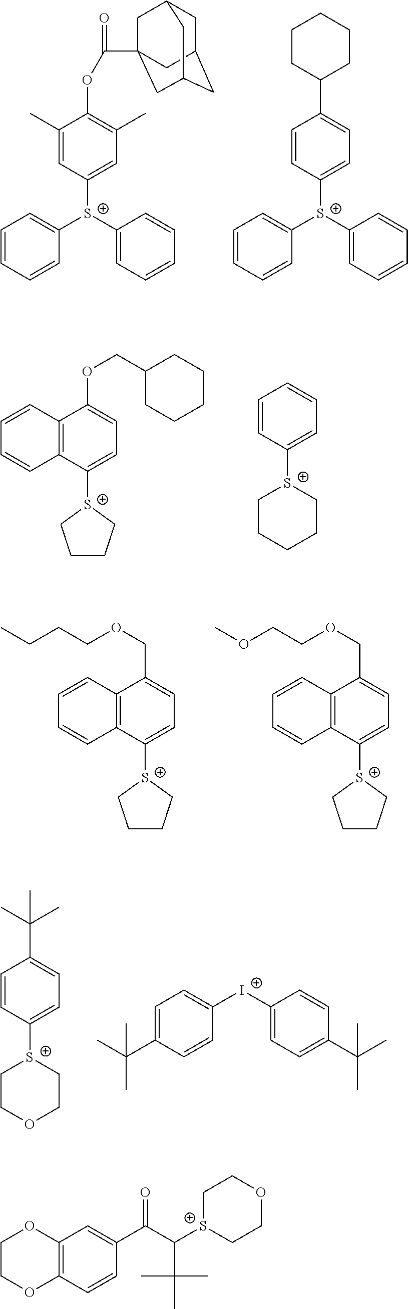

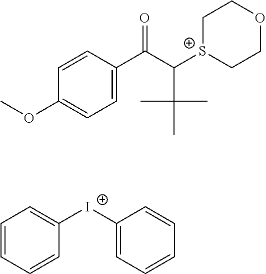 [SMILES: COc1ccc(C(=O)C([S+]2CCOCC2)C(C)(C)C)cc1 ; c1ccc([I+]c2ccccc2)cc1]

Preferred examples of the anion Z⁻ in Formula ZI and Formula ZII, Zc⁻ in Formula ZI-3, and Z⁻ in Formula ZI-4 are shown below.

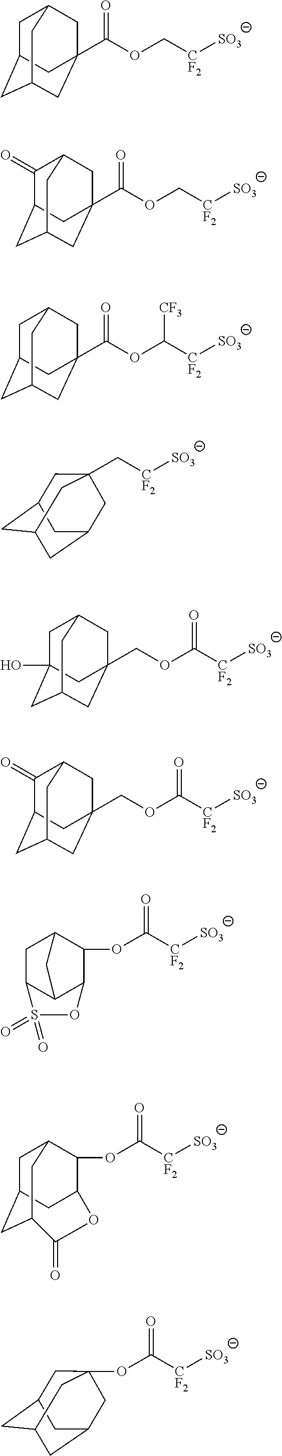

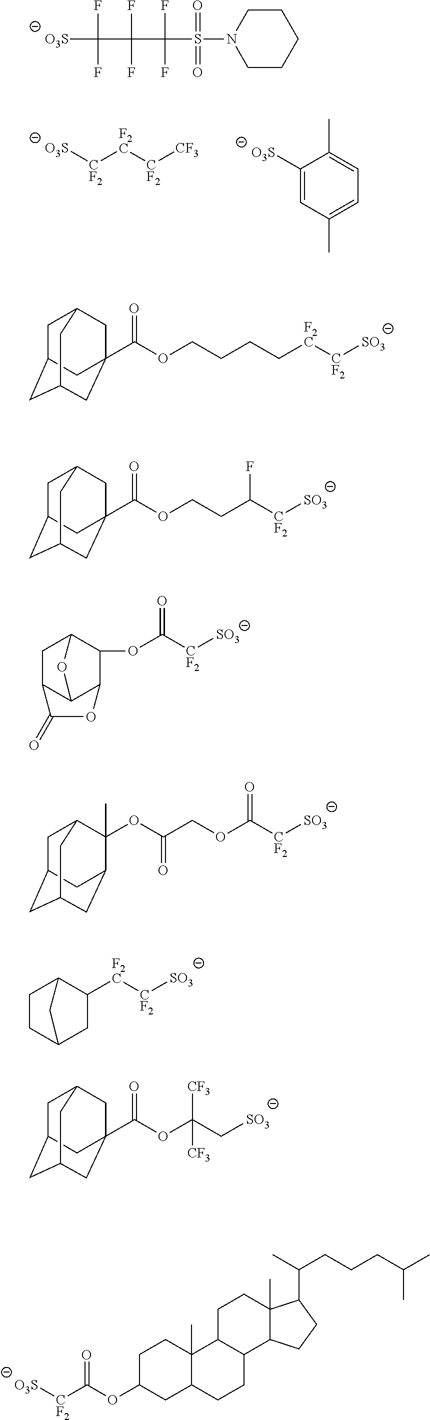

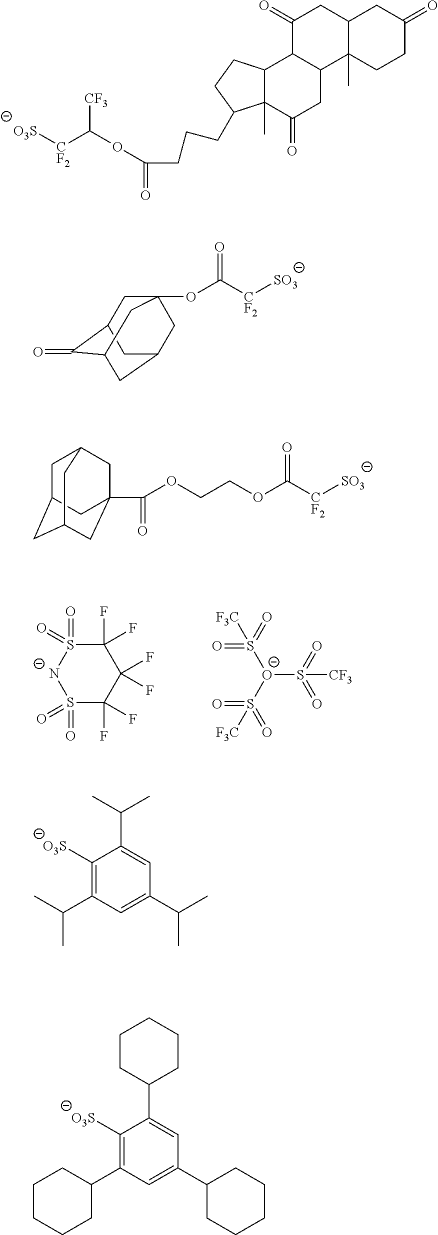

The cation and the anion can be optionally combined and used as a photoacid generator.

Among those, it is preferable that the photoacid generator is an ionic compound including a cation and an anion, and the anion includes an ion represented by any one of Formula An-1, Formula An-2, or Formula An-3.

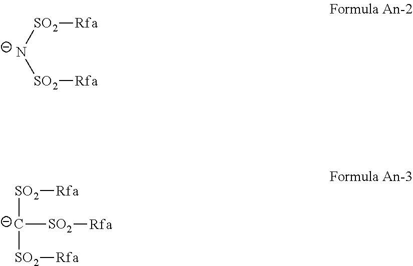

In Formula An-2 and Formula An-3, Rfa's each independently represent a monovalent organic group having a fluorine atom, and a plurality of Rfa's may be bonded to each other to form a ring.

Rfa is preferably an alkyl group substituted with at least one fluorine atom. The number of carbon atoms of this alkyl group preferably 1 to 10, and more preferably 1 to 4. In addition, the alkyl group substituted with at least one fluorine atom is preferably a perfluoroalkyl group.

In addition, it is preferable that a plurality of Rfa's are bonded to each other to form a ring.

The photoacid generator may be in a form of a low-molecular-weight compound or in a form incorporated into a part of a polymer. Further, the form of a low-molecular-weight compound and the form incorporated into a part of a polymer may also be used in combination.

The photoacid generator is preferably in the form of the low-molecular-weight compound.

In a case where the photoacid generator is in the form of the low-molecular-weight compound, the molecular weight is preferably 3,000 or less, more preferably 2,000 or less, and still more preferably 1,000 or less.

In a case where the photoacid generator is in the form incorporated into a part of a polymer, it may be incorporated into the above-mentioned resin (A) or into a resin other than the resin (A).

The photoacid generators may be used singly or in combination of two or more kinds thereof.

The content of the photoacid generator (the total content in a case where a plurality of the photoacid generators are present) in the composition is preferably 0.1% by mass to 35% by mass, more preferably 0.5% by mass to 25% by mass, still more preferably 3% by mass to 20% by mass, and particularly preferably 3% by mass to 15% by mass, with respect to the total solid contents of the composition.

In a case where the compound represented by Formula ZI-3 or Formula ZI-4 is included as the photoacid generator, the content of the photoacid generator (the total content in a case where a plurality of the photoacid generators are present) included in the composition is preferably 5% by mass to 35% by mass, and more preferably 7% by mass to 30% by mass, with respect to the total solid contents of the composition.

<Solvent>

The photosensitive resin composition according to the present disclosure includes a solvent.

In the photosensitive resin composition according to the present disclosure, a known resist solvent can be appropriately used. For example, the known solvents disclosed in paragraphs 0665 to 0670 of US2016/0070167A1, paragraphs 0210 to 0235 of US2015/0004544A1, paragraphs 0424 to 0426 of US2016/0237190A1, and paragraphs 0357 to 0366 of US2016/0274458A1 can be suitably used.

Examples of the solvent which can be used in the preparation of the composition include organic solvents such as alkylene glycol monoalkyl ether carboxylate, alkylene glycol monoalkyl ether, alkyl lactate ester, alkyl alkoxypropionate, a cyclic lactone (preferably having 4 to 10 carbon atoms), a monoketone compound (preferably having 4 to 10 carbon atoms) which may have a ring, alkylene carbonate, alkyl alkoxyacetate, and alkyl pyruvate.

A mixed solvent obtained by mixing a solvent containing a hydroxyl group in the structure and a solvent containing no hydroxyl group in the structure may be used as the organic solvent.

As the solvent containing a hydroxyl group and the solvent containing no hydroxyl group, the above-mentioned exemplary compounds can be appropriately selected, but as the solvent containing a hydroxyl group, an alkylene glycol monoalkyl ether, alkyl lactate, or the like is preferable, and propylene glycol monomethyl ether (PGME), propylene glycol monoethyl ether (PGEE), methyl 2-hydroxyisobutyrate, or ethyl lactate is more preferable. Further, as the solvent containing no hydroxyl group, an alkylene glycol monoalkyl ether acetate, alkyl alkoxy propionate, a monoketone compound which may have a ring, cyclic lactone, alkyl acetate, or the like is preferable; among these, propylene glycol monomethyl ether acetate (PGMEA), ethyl ethoxypropionate, 2-heptanone, γ-butyrolactone, cyclohexanone, cyclopentanone, or butyl acetate is more preferable; and propylene glycol monomethyl ether acetate, γ-butyrolactone, ethyl ethoxypropionate, cyclohexanone, cyclopentanone, or 2-heptanone is still more preferable. As the solvent containing no hydroxyl group propylene carbonate is also preferable. Among those, the solvent particularly preferably includes γ-butyrolactone from the viewpoint of the evenness of a layer thus formed.

The mixing ratio (mass ratio) of the solvent containing a hydroxyl group to the solvent containing no hydroxyl group is 1/99 to 99/1, preferably 10/90 to 90/10, and more preferably 20/80 to 60/40. A mixed solvent containing 50% by mass or more of the solvent containing no hydroxyl group is preferable from the viewpoint of coating evenness.

The solvent preferably includes propylene glycol monomethyl ether acetate, and may be a single solvent formed of propylene glycol monornethyl ether acetate or a mixed solvent of two or more kinds of solvents containing propylene glycol monomethyl ether acetate.

The concentration of the solid contents of the photosensitive resin composition according to the present disclosure is not particularly limited, and is preferably 1.0% by mass to 10% by mass, more preferably 2.0% by mass to 5.7% by mass, and still more preferably 2.0% by mass to 5.3% by mass.

<Hydrophobic Resin>

The photosensitive resin composition according to the present disclosure preferably contains a hydrophobic resin (also referred to as a “hydrophobic resin (E)”. Further, the hydrophobic resin (E) is preferably a resin which is different from the resin (A) and the resin (B).

By incorporating the hydrophobic resin (E) into the photosensitive resin composition according to the present disclosure, it is possible to control the static/dynamic contact angle at a surface of an actinic ray-sensitive or radiation-sensitive film. Thus, it becomes possible to improve development characteristics, suppress generation of out gas, improve immersion liquid tracking properties upon liquid immersion exposure, and reduce liquid immersion defects, for example.

It is preferable that the hydrophobic resin (E) is designed to be unevenly distributed on a surface of a resist film, but unlike the surfactant, the hydrophobic resin (E) does not necessarily have a hydrophilic group in a molecule thereof and does not necessarily contribute to uniform mixing of polar/non-polar materials.

In addition, in the present disclosure, the resin having a fluorine atom is intended to be regarded to a hydrophobic resin and a fluorine-containing resin which will be described later. Further, it is preferable that the resin having a constitutional unit having an acid-decomposable group does not have a fluorine atom.

The hydrophobic resin (E) is preferably a resin including a constitutional unit having at least one selected from the group consisting of a “fluorine atom”, a “silicon atom”, and a “CH₃ partial structure which is contained in a side chain moiety of a resin” from the viewpoint of uneven distribution on a film surface layer.

In a case where the hydrophobic resin (E) includes a fluorine atom or a silicon atom, the fluorine atom or the silicon atom described above in the hydrophobic resin (E) may be included in the main chain of a resin or may be included in a side chain.

It is preferable that the hydrophobic resin (E) has at east one group selected from the following group of (x) to (z):

(x) an acid group,

(y) a group whose solubility in an alkali developer increases through decomposition by the action of the alkali developer (hereinafter also referred to as a polarity converting group), and

(z) a group that decomposes by the action of an acid.

Examples of the acid group (x) include a phenolic hydroxyl group, a carboxylic acid group, a fluorinated alcohol group, a sulfonic acid group, a sulfonamido group, a sulfonylimido group, an (alkylsulfonyl)(alkylcarbonyl)methylene group, an (alkylsulfonyl)(alkylcarbonyl)imido group, a bis (alkylcarbonyl)methylene group, a bis (alkylcarbonyl)imido group, a bis (alkylsulfonyl)methylene group, a bis (alkylsulfonyl)imido group, a tris (alkylcarbonyl)methylene group, and a tris (alkvlsulfonyl)methylene group.

As the acid group, a fluorinated alcohol group (preferably hexafluoroisopropanol group), a sulfonimido group, or a bis (alkylcarbonyl)methylene group is preferable.

Examples of the group (y) whose solubility in an alkali developer increases through decomposition by the action of the alkali developer include a lactone group, a carboxylic acid ester group (—COO—), an acid anhydride group (—C(O)OC(O)—), an acid imido group (—NHCONH—), a carboxylic acid thioester group (—COS—), a carbonic acid ester group (—OC(O)O—), a sulfuric acid ester group (—OSO—₂O—), and a sulfonic acid ester group (—SO₂O—), and the lactone group or the carboxylic acid ester group (—COO—) is preferable.

Examples of the constitutional units including these groups include a constitutional unit in which the group is directly bonded to the main chain of a resin, such as a constitutional unit with an acrylic acid ester or a methacrylic acid ester. In this constitutional unit, the group may be bonded to the main chain of the resin through a linking group. Alternatively, the constitutional unit may also be incorporated into a terminal of the resin by using a polymerization initiator or chained transfer agent having the group during polymerization.

Examples of the constitutional unit having a lactone group include the same ones as the constitutional unit having a lactone structure as described earlier in the section of the resin (A).

The content of the constitutional unit having a group (y) whose solubility in an alkali developer increases through decomposition by the action of the alkali developer is preferably 1% to 100% by mole, more preferably 3% to 98% by mole, and still more preferably 5% to 95% by mole, with respect to all the constitutional units in the hydrophobic resin (E).

With respect to the hydrophobic resin (E), examples of the constitutional unit having a group (z) that decomposes by the action of an acid include the same ones as the constitutional units having an acid-decomposable group, as mentioned in the resin (A). The constitutional unit having a group (z) that decomposes by the action of an acid may have at least one of a fluorine atom or a silicon atom. The content of the constitutional units having a group (z) that decomposes by the action of an acid is preferably 1% by mole to 80% by mole, more preferably 10% by mole to 80% by mole, and still more preferably 20% by mole to 60% by mole, with respect to all the constitutional units in the resin (E).

The hydrophobic resin (E) may further have a constitutional unit which is different from the above-mentioned constitutional units.

The content of the constitutional units including a fluorine atom is preferably 10% by mole to 100% by mole, and more preferably 30% by mole to 100% by mole, with respect to all the constitutional units included in the hydrophobic resin (E). Further, the content of the constitutional units including a silicon atom is preferably 10% by mole to 100% by mole, and more preferably 20% by mole to 100% by mole, with respect to all the constitutional units included in the hydrophobic resin (E).

On the other hand, in a case where the hydrophobic resin (E) includes a CH₃ partial structure in the side chain moiety thereof, it is also preferable that the hydrophobic resin (E) has a form not having substantially any one of a fluorine atom and a silicon atom. Further, it is preferable that the hydrophobic resin (E) is substantially constituted with only constitutional units, which are composed of only atoms selected from a carbon atom, an oxygen atom, a hydrogen atom, a nitrogen atom, and a sulfur atom.

The weight-average molecular weight of the hydrophobic resin (E) in terms of standard polystyrene is preferably 1,000 to 100,000, and more preferably 1,000 to 50,000.

The total content of residual monomers and oligomer components included in the hydrophobic resin (E) is preferably 0.01% by mass to 5% by mass, and more preferably 0.01% by mass to 3% by mass. Further, the dispersity (Mw/Mn) is preferably in the range of 1 to 5, and more preferably in the range of I to 3.

As the hydrophobic resin (E), known resins can be appropriately selected and used singly or as a mixture. For example, the known resins disclosed in paragraphs 0451 to 0704 of US2015/0168830A1 and paragraphs 0340 to 0356 of US2016/0274458A1 can be suitably used as the hydrophobic resin (E). Further, the constitutional units disclosed in paragraphs 0177 to 0258 of US2016/0237190A1 are also preferable as a constitutional unit constituting the hydrophobic resin (E).

[Fluorine-Containing Resin]

The hydrophobic resin (E) is preferably a resin including a fluorine atom (also referred to as a fluorine-containing resin).

In a case where the hydrophobic resin (E) includes a fluorine atom, it is preferable that the hydrophobic resin (E) is a resin having an alkyl group having a fluorine atom, a cycloalkyl group having a fluorine atom, or an aryl group having a fluorine atom as a partial structure having a fluorine atom.

The alkyl group having a fluorine atom is a linear or branched alkyl group, in which at least one hydrogen atom is substituted with a fluorine atom, preferably has 1 to 10 carbon atoms, and more preferably has 1 to 4 carbon atoms.

The cycloalkyl group having a fluorine atom is a monocyclic or polycyclic cycloalkyl group in which at least one hydrogen atom is substituted with a fluorine atom.

The aryl group having a fluorine atom is an aryl group such as a phenyl group and a naphthyl group, in which at least one hydrogen atom is substituted with a fluorine atom.

As each of the alkyl group having a fluorine atom, the cycloalkyl group having a fluorine atom, and the aryl group having a fluorine atom, groups represented by Formulae F2 to F4 are preferable.

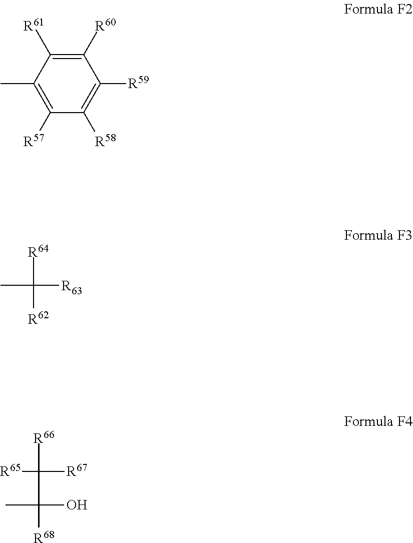

In Formulae F2 to F4,

R⁵⁷ to R⁶⁸ each independently represent a hydrogen atom, a fluorine atom, or an (linear or branched) alkyl group. It should be noted that at least one of R⁵⁷, . . . , or R⁶¹, at least one of R⁶², . . . , or R⁶⁴, and at least one of R⁶⁵, . . . , or R⁶⁸ each independently represent a fluorine atom or an alkyl group in which at least one hydrogen atom is substituted with a fluorine atom.

It is preferable that all of R⁵⁷ to R⁶¹ and R⁶⁵ to R⁶⁷ are fluorine atoms. R⁶² R⁶³, and R⁶⁸ are each preferably an alkyl group (preferably having 1 to 4 carbon atoms) in which at least one hydrogen atom is substituted with a fluorine atom, and more preferably a perfluoroalkyl group having 1 to 4 carbon atoms. R⁶² and R⁶³ may be linked to each other to form a ring.

Among those, from the viewpoint that the effects according to the present disclosure are more excellent, it is preferable that the fluorine-containing resin has alkali decomposability.

The expression, the fluorine-containing resin having alkali decomposability, means that after 10 minutes from adding 100 mg of a fluorine-containing resin to a mixed liquid of 2 mL of a buffer solution at pH 10 and 8 mL of THF and leaving the mixture to stand at 40° C., 30% by mole or more of the total amount of the decomposable groups in the fluorine-containing resin is hydrolyzed. Further, the decomposition rate can be calculated from a ratio of the raw materials to the decomposed products by means of NMR analysis.

From the viewpoints of improvement of the tolerance of a depth of focus, the pattern linearity, and the development characteristics, suppression of generation of out gas, improvement of immersion liquid tracking properties upon liquid immersion exposure, and reduction in liquid immersion defects, it is preferable that the fluorine-containing resin has a constitutional unit represented by Formula X.

Furthermore, from the viewpoints of improving the tolerance of a depth of focus, the pattern linearity, and the development characteristics, suppressing the generation of out gas, improving immersion liquid tracking properties upon liquid immersion exposure, and reducing the liquid immersion defects, it is preferable that the photosensitive resin composition according to the present disclosure further includes a fluorine-containing resin having the constitutional unit represented by Formula X.

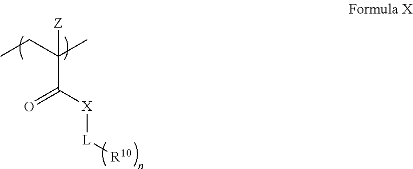

In Formula X, Z represents a halogen atom, a group represented by R¹¹OCH₂—, or a group represented by R¹²OC(═O)CH₂—, R¹¹ and R¹² each independently represent a substituent, and X represents an oxygen atom or a sulfur atom. L represents an (n+1)-valent linking group. R¹⁰ represents a group having a group whose solubility of the fluorine-containing resin in an aqueous alkali solution increases through decomposition by the action of the aqueous alkali solution, n represents a positive integer, and in a case where n is 2 or more, a plurality of R's may be the same as or different from each other.

Examples of the halogen atom of Z include a fluorine atom, a chlorine atom, a bromine atom, and an iodine atom, and the fluorine atom is preferable.

Examples of the substituent as each of the R¹¹ and R¹² include an alkyl group (preferably having 1 to 4 carbon atoms), a cycloalkyl group (preferably having 6 to 10 carbon atoms), and an aryl group (preferably having 6 to 10 carbon atoms). Further, the substituent as each of R¹¹ and R¹² may further have a substituent, and examples of such additional substituent include an alkyl group (preferably having 1 to 4 carbon atoms), a halogen atom, a hydroxyl group, an alkoxy group (preferably having 1 to 4 carbon atoms), and a carboxyl group.

The linking group as L is preferably a divalent or trivalent linking group (in other words, n is preferably 1 or 2), and more preferably the divalent linking group (in other words, n is preferably 1). The linking group as L is preferably a linking group selected from the group consisting of an aliphatic group, an aromatic group, and a combination thereof.

For example, in a case where n is 1 and the linking group as L is a divalent linking group, examples of the divalent aliphatic group include an alkylene group, an alkenylene group, an alkynylene group, and a polyalkyleneoxy group. Among those, the alkylene group or the alkenylene group is preferable, and the alkylene group is more preferable.

The divalent aliphatic group may have either a chained structure or a cyclic structure, but preferably has the chained structure rather than the cyclic structure, and preferably has a linear structure rather than the branch-chained structure. The divalent aliphatic group may have a substituent and examples of the substituent include a halogen atom (a fluorine atom, a chlorine atom, a bromine atom, or an iodine atom), a hydroxyl group, a carboxyl group, an amino group, a cyano group, an aryl group, an alkoxy group, an aryloxy group, an acyl group, an alkoxycarbonyl group, an aryloxycarbonyl group, an acyloxy group, a monoalkylamino group, a dialkylamino group, an arylamino group, and a diarylamino group.

Examples of the divalent aromatic group include an amylene group. Among those, the phenylene group and the naphthylene group are preferable.

The divalent aromatic group may have a substituent, and examples of the substituent include an alkyl group, in addition to the examples of the substituent with regard to the divalent aliphatic group.

In addition, L may be a divalent group formed by removing two or more hydrogen atoms at any position from the structure represented by each of Formula LC1-1 to Formula LC1-21 or Formula SL1-1 to Formula SL1-3 as described above.

In a case where n is 2 or more, specific examples of the (n+1)-valent linking group include groups formed by removing any (n-1) hydrogen atoms from the specific examples of the divalent linking group as described above.

Specific examples of L include the following linking groups.

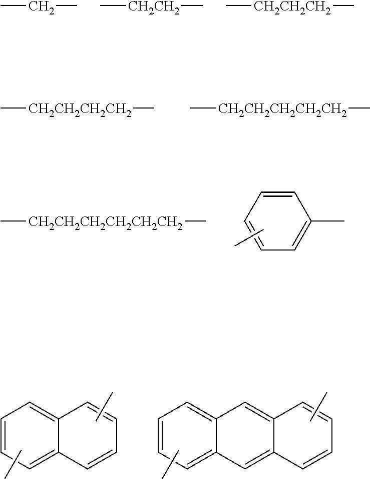

Moreover, these linking groups may further have a substituent as described above.

As R¹⁰, a group represented by Formula W is preferable.

—Y—R²⁰ Formula W

In Formula W, Y represents a group whose solubility in an aqueous alkali solution of a fluorine-containing resin increases through decomposition by the action of the aqueous alkali solution. R²⁰ represents an electron-withdrawing group.

Examples of Y include a carboxylic acid ester group (—COO— or OCO—), an acid anhydride group (—C(O)OC(O)—), an acid imido group (—NHCONH—), a carboxylic acid thioester group (—COS—), a carbonic acid ester group (—OC(O)O—), a sulfuric acid ester group (—OSO₂O—), and a sulfonic acid ester group (—SO₂O—), and the carboxylic acid ester group is preferable.

As the electron-withdrawing group, a partial structure represented by Formula EW is preferable. *in Formula EW represents the number of bonds directly linked to a group Y in Formula W.

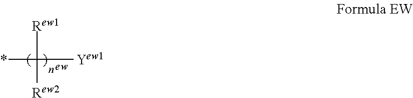

In Formula EW,

n^{ew} is a repetition number of the linking groups represented by —C(R^{ew1})(R^{ew2})— and represents an integer of 0 or 1. A case where n^{ew} is 0 indicates that the bonding is formed by a single bond and Y^{ew1} is directly bonded.

Examples of Y^{ew1} include a halogen atom, a cyano group, a nitro group, a halo(cyclo)alkyl group or haloaryl group represented by —C(R^{f1})(R^{f2})—R^{f3} which will be described later, an oxy group, a carbonyl group, a sulfonyl group, a sulfinyl group, and a combination thereof. It should be noted that in a case where Y^{ew1} is a halogen atom, a cyano group, or a nitro group, n^{ew} is 1.

R^{ew1} and R^{ew2} each independently represent any group, and examples of the group include a hydrogen atom, an alkyl group (preferably having 1 to 8 carbon atoms), a cycloalkyl group (preferably having 3 to 10 carbon atoms), or an aryl group (preferably having 6 to 10 carbon atoms).

At least two of R^{ew1}, R^{ew2}, or Y^{ew1} may be linked to each other to form a ring.

In addition, the “halo(cyclo)alkyl group” represents an alkyl group or cycloalkyl group which is at least partially halogenated, and the “haloaryl group” represents an aryl group which is at least partially halogenated.

As Y^{ew1}, a halogen atom, a halo(cyclo)alkyl group represented by —C(R^{f1})(R^{f2})—R^{f3}, or a haloaryl group is preferable.

R^{f1} represents a halogen atom, a perhaloalkyl group, a perhalocycloalkyl group, or a perhaloaryl group, and is preferably a fluorine atom, a perfluoroalkyl group, or a perfluorocycloalkyl group, and more preferably the fluorine atom or a trifluoromethyl group.

R^{f2} and R^{f3} each independently represent a hydrogen atom, a halogen atom, or an organic group, and R^{f2} and R^{f3} may be linked to each other to form a ring. Examples of the organic group include an alkyl group, a cycloalkyl group, and an alkoxy group, and these may be substituted with a halogen atom (preferably a fluorine atom). It is preferable that R^{f2} and R^{f3} are each a (halo)alkyl group or a (halo)cycloalkyl group. It is more preferable that R^{f2} represents the same group as R^{f1} or is linked to R^{f3} to form a ring.

Examples of the ring formed by the linking of R^{f2} and R^{f3} include a cycloalkyl ring.

The (halo)alkyl group in each of R^{f1} to R^{f3} may be linear or branched, and the linear (halo)alkyl group preferably has 1 to 30 carbon atoms, and more preferably 1 to 20 carbon atoms.

The (halo)cycloalkyl group in each of R^{f1} to R^{f3}, or the ring formed by the linking of R^{f2} and R^{f3} may be monocyclic or polycyclic. In a case where the (halo)cycloalkyl group is polycyclic, the (halo)cycloalkyl group may be bridged. That is, in this case, the (halo)cycloalkyl group may have a crosslinked structure.

Examples of these (halo)cycloalkyl groups include those represented by the following formulae, and groups formed by halogenating the groups. Further, some of carbon atoms in the cycloalkyl group may be substituted with heteroatoms such as an oxygen atom.

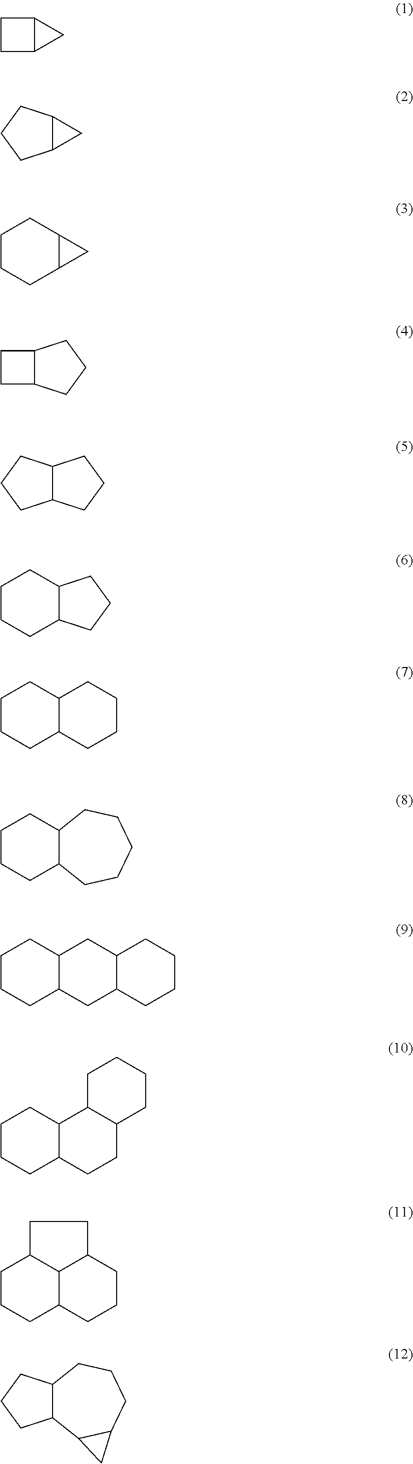

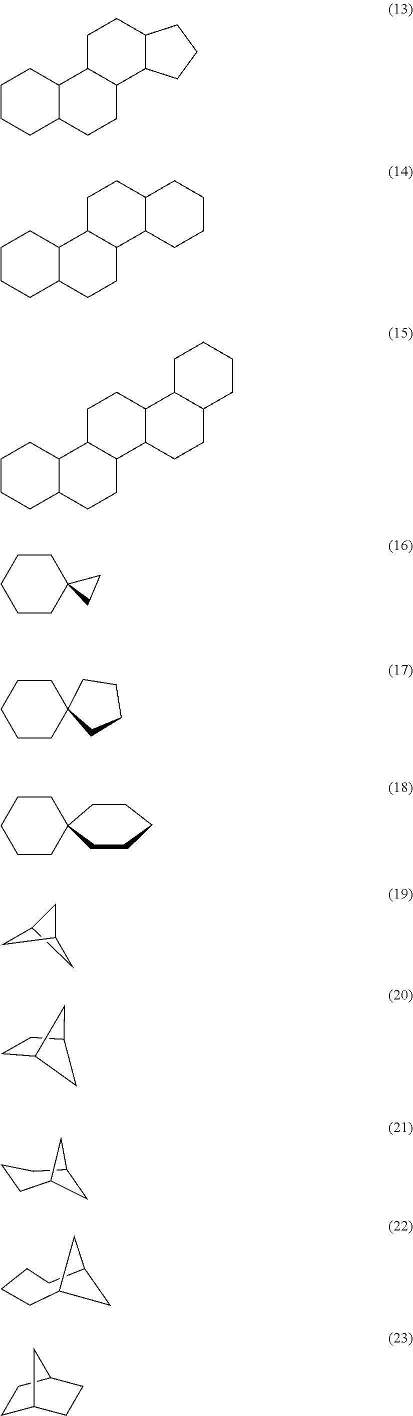

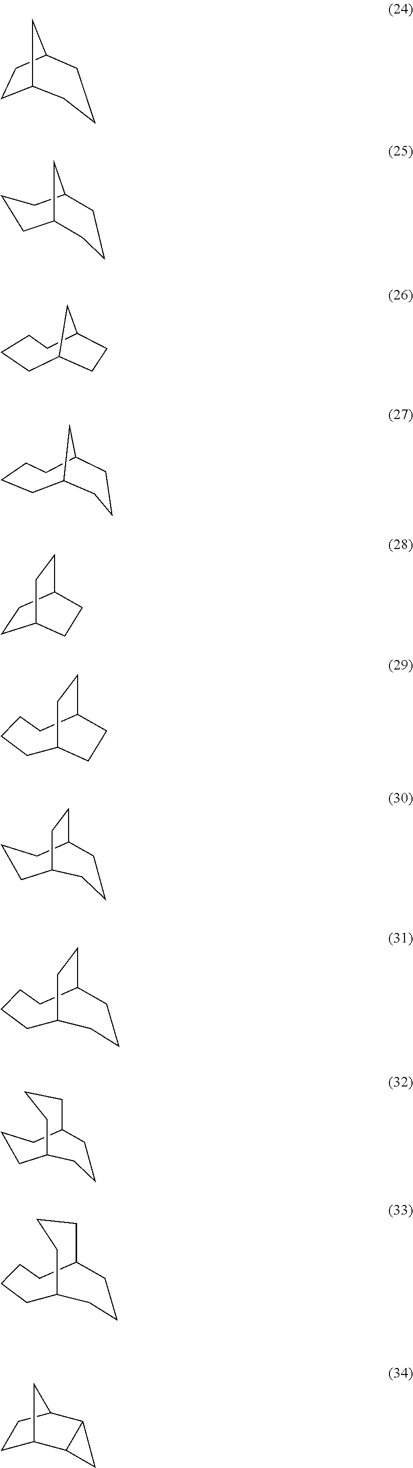

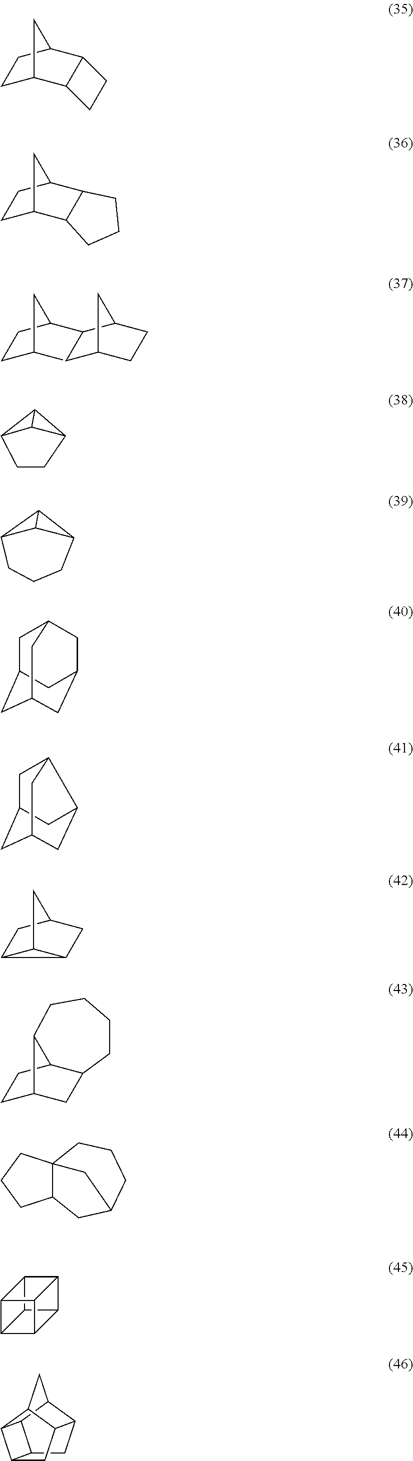

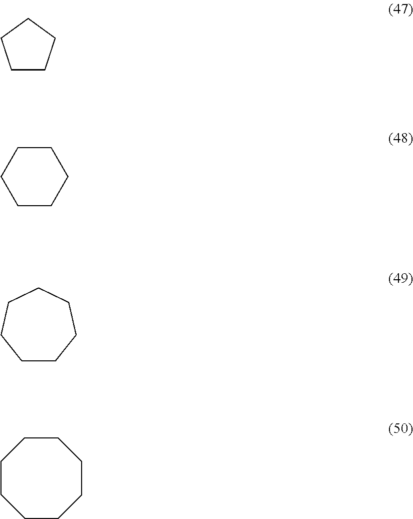 [SMILES: C1CCCC1 ; C1CCCCC1 ; C1CCCCCC1 ; C1CCCCCCC1]

As the (halo)cycloalkyl group in each of R^{f2} and R^{f3}, or a ring formed by the linking of R^{f2} and R^{f3}, a fluorocycloalkyl group represented by —C₍ₙ₎F₍₂ₙ₋₂₎H is preferable. Here, the number of carbon atoms, n, is not particularly limited, but is preferably 5 to 13, and more preferably 6.

Examples of the (per)haloaryl group in Y^{ew1} or R^{f1} include a perfluoroaryl group represented by —C₍ₙ₎F₍ₙ₋₁₎. Here, the number of carbon atoms, n, is not particularly limited, but is preferably 5 to 13, and more preferably 6.

As a ring formed by the mutual linking of at least two of R^{ew1}, R^{ew2}, or Y^{ew1}, a cycloalkyl group or a heterocyclic group is preferable.

Each of the groups and the rings constituting the partial structure represented by Formula EW may further have a substituent.

In Formula W, R²⁰ is preferably an alkyl group substituted with one or more selected from the group consisting of a halogen atom, a cyano group, and a nitro group, more preferably an alkyl group substituted with a halogen atom (haloalkyl group), and still more preferably a fluoroalkyl group. The alkyl group substituted with one or more selected from the group consisting of a halogen atom, a cyano group, and a nitro group preferably has 1 to 10 carbon atoms, and more preferably 1 to 5 carbon atoms.

More specifically, R²⁰ is preferably an atomic group represented by —C(R′¹)(R′^{f1})(R′^{f2}) or —C(R′¹)(R′²)(R′^{f1}). R′¹ and R′² each independently represent a hydrogen atom or an alkyl group not substituted with an electron-withdrawing group (preferably an unsubstituted alkyl group). R′^{f1} and R′^{f2} each independently represent a halogen atom, a cyano group, a nitro group, or a perfluoroalkyl group.

The alkyl group as each of R′¹ and R′² may be linear or branched, and preferably has 1 to 6 carbon atoms.

The perfluoroalkyl group as each of R′^{f1} and R′^{f2} may be linear or branched, and preferably has 1 to 6 carbon atoms.

Specific preferred examples of R²⁰ include —CF₃, —C₂F₅, —C₃F₇, —C₄F₉, —CF(CF₃)₂, —CF(CF₃)C₂F₅, —CF2CF(CF₃)₂, —C(CF₃)₃, —C₅F₁₁, —C₆F₁₃, —C₇F₁₅, —C₈F₁₇, —CH₂CF₃, —CH₂C₂F₅, —CH₂C₃F₇, —CH(CF₃)₂, —CH(CF₃)C₂F₅, —CH₂CF(CF₃)₂, and —CH₂CN. Among those, —CF₃, —C₂F₅, —C₃F₇, —C₄F₉, —CH₂CF₃, —CH₂C₂F₅, —CH₂C₃F₇, —CH(CF₃)₂, or —CH₂CN is preferable, —CH₂CF₃, —CH₂C₂F₅, —CH₂C₃F₇, —CH(CF₃)₂, or —CH₂CN is more preferable, —CH₂C₂F₅, —CH(CF₃)₂, or —CH₂CN is still more preferable, and —CH₂C₂F₅ or —CH(CF₃)₂ is particularly preferable.

As the constitutional unit represented by Formula X, a constitutional unit represented by Formula X-1 or Formula X-2 is preferable, and the constitutional unit represented by Formula X-1 is more preferable.

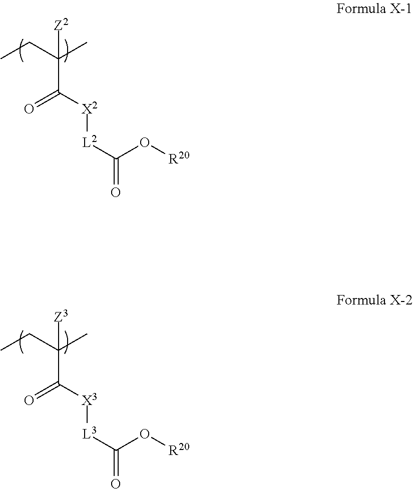

In Formula X-1, R²⁰ represents an electron-withdrawing group, L² represents a divalent linking group, X² represents an oxygen atom or a sulfur atom, and Z² represents a halogen atom.

In Formula X-2, R²⁰ represents an electron-withdrawing group, L³ represents a divalent linking group, X³ represents an oxygen atom or a sulfur atom, and Z³ represents a halogen atom.

Specific examples and preferred examples of the divalent linking group of each of L² and L³ include the same ones as described in L as the divalent linking group in Formula X.

The electron-withdrawing group as each of R² and R³ is preferably the partial structure represented by Formula EW, specific examples and preferred examples thereof are the same as described above, but the halo(cyclo)alkyl group is more preferable.

In Formula X-1, L² and R² are not bonded to each other to form a ring in any case, and in Formula X-2, L³ and R³ are not bonded to each other to form a ring in any case.

X² and X³ are each preferably an oxygen atom.

As each of Z² and Z³, a fluorine atom or a chlorine atom is preferable, and the fluorine atom is more preferable.

In addition, as the constitutional unit represented by Formula X, a constitutional unit represented by Formula X-3 is also preferable.

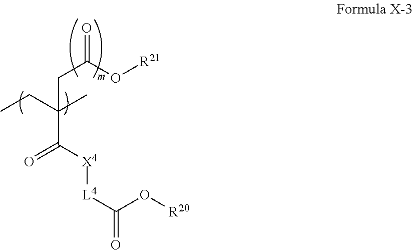

In Formula X-3, R²⁰ represents an electron-withdrawing group, R²¹ represents a hydrogen atom, an alkyl group, or an aryl group, L⁴ represents a divalent linking group, X₄ represents an oxygen atom or a sulfur atom, and m represents 0 or 1.

Specific examples and preferred examples of the divalent linking group of L⁴ include the same ones as described in L as the divalent linking group in Formula X).

The electron-withdrawing group as R⁴ is preferably the partial structure represented by Formula EW, specific examples and preferred examples thereof are the same as described above, but the halo(cyclo)alkyl group is more preferable.

Furthermore, in Formula X-3, L⁴ and R⁴ are not bonded to each other to form a ring in any case.

As X⁴, an oxygen atom is preferable.

Moreover, as the constitutional unit represented by Formula X, a constitutional unit represented by Formula Y-1 or a constitutional unit represented by Formula Y-2 is also preferable.

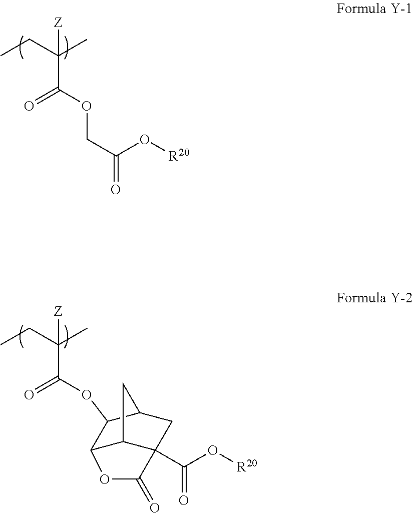

In Formula Y-1 and Formula Y-2, Z represents a halogen atom, a group represented by R¹¹OCH₂—, or a group represented by R¹²OC(═O)CH₂—, R¹¹ and R¹² each independently represent a substituent, and R²⁰ represents an electron-withdrawing group.

The electron-withdrawing group as R²⁰ is preferably the partial structure represented by Formula EW, specific examples and preferred examples thereof are the same as described above, but the halo(cyclo)alkyl group is more preferable.

Specific examples and preferred examples of the halogen atom, the group represented by R¹¹OCH₂—, or the group represented by R¹²OC(═O)CH₂— as Z include the same ones as described in Formula 1.

The content of the constitutional unit represented by Formula X is preferably 10% by mole to 100% by mole, more preferably 20% by mole to 100% by mole, and still more preferably 30% by mole to 100% by mole, with respect to all the constitutional units of the uorine-containing resin.

Preferred examples of the constitutional unit constituting the hydrophobic resin (E) are shown below.

Preferred examples of the hydrophobic resin (E) include resins formed by optionally combining these constitutional units or resins E-1 to E-11 used in Examples, but are not limited thereto.

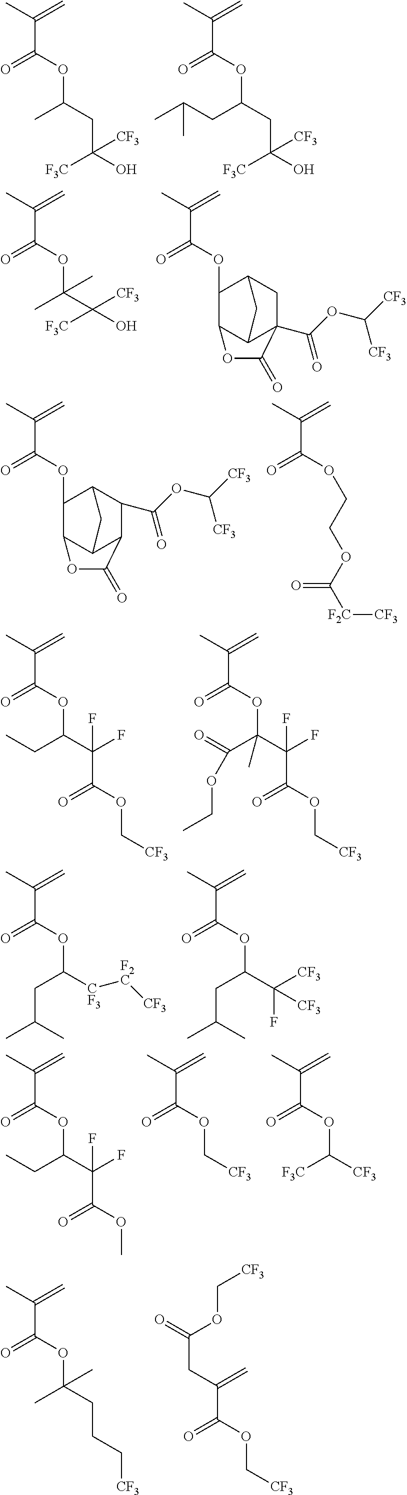

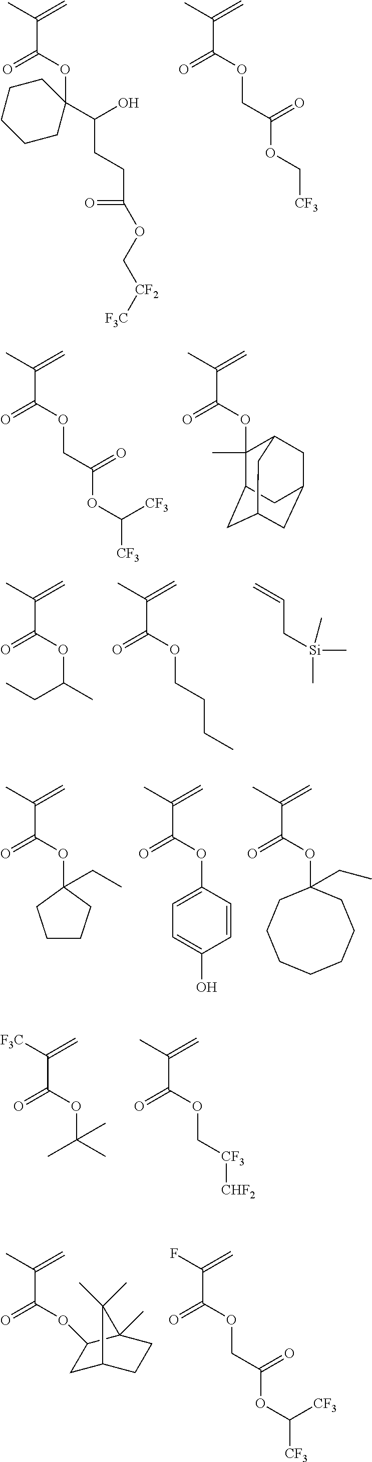

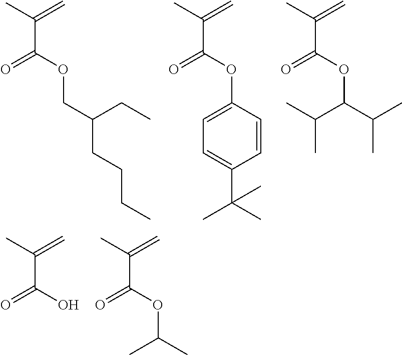

Moreover, specific examples of the fluorine-containing resin and the repeating units which can be included in the fluorine-containing resin are shown below. In the following tables, the compositional ratios of the constitutional units represent molar ratios. Further, the constitutional units in the compositions described in the following tables will be described later (TMS represents a trimethylsilyl group). In the tables, Pd represents the dispersity (Mw/Mn) of the fluorine-containing resin.

TABLE 1

| | Composition | | |
|---|---|---|---|
| Resin | (Compositional ratio) | Mw | Pd |
| P-1 | P-1 (100) | 7,500 | 1.6 |
| P-2 | p-4 (100) | 11,000 | 1.5 |
| P-3 | p-7/p-60 (90/10) | 12,000 | 1.6 |
| P-4 | p-13/p-60 (80/20) | 9,000 | 1.6 |
| P-5 | p-12 (100) | 9,000 | 1.6 |
| P-6 | p-30 (100) | 7,000 | 1.5 |
| P-7 | p-47/p-63 (90/10) | 8,000 | 1.6 |
| P-8 | p-50 (100) | 9,000 | 1.5 |
| P-9 | p-45 (100) | 10,000 | 1.6 |
| P-10 | p-3/p-38 (90/10) | 11,000 | 1.5 |
| P-11 | p-20 (100) | 12,000 | 1.6 |
| P-12 | p-16/p-62 (90/10) | 12,000 | 1.4 |
| P-13 | p-23 (100) | 11,500 | 1.6 |
| P-14 | p-27 (100) | 15,000 | 1.5 |
| P-15 | p-57 (100) | 8,500 | 1.6 |
| P-16 | p-3/p-39 (98/2) | 17,0,0 | 1.4 |
| P-17 | p-8 (100) | 12,000 | 1.4 |
| P-18 | p-17 (100) | 15,000 | 1.6 |
| P-19 | p-40/p-65 (98/2) | 10,000 | 1.6 |
| P-20 | p-42 (100) | 8,500 | 1.5 |
| P-21 | p-42/p-61 (80/20) | 9,000 | 1.6 |
| P-22 | p-1/p-63 (90/10) | 11,000 | 1.6 |
| P-23 | p-41 (100) | 16,000 | 1.6 |
| P-24 | p-11 (100) | 14,500 | 1.6 |
| P-25 | p-43 (100) | 12,500 | 1.6 |
| P-26 | p-54 (100) | 12,000 | 1.4 |
| P-27 | p-54/p-38 (80/20) | 13,500 | 1.6 |
| P-28 | p-42/p-61 (80/20) | 9,000 | 1.6 |
| P-29 | p-18 (100) | 10,000 | 1.6 |
| P-30 | p-23/p-63 (98/2) | 95,000 | 1.4 |
| P-31 | p-20/p-64 (90/10) | 8,000 | 1.6 |
| P-32 | p-10 (100) | 9,500 | 1.6 |
| P-33 | p-5/p-69 (80/20) | 12,000 | 1.6 |
| P-34 | p-44 (100) | 10,000 | 1.6 |
| P-35 | p-44/p-71 (98/2) | 9,500 | 1.6 |
| P-36 | p-44/p-64 (90/10) | 15,000 | 1.6 |
| P-37 | p-54/p-74 (70/30) | 16,000 | 1.6 |
| P-38 | p-55 (100) | 17,000 | 1.6 |
| P-39 | p-25 (100) | 9,500 | 1.6 |
| P-40 | p-29 (100) | 9,000 | 1.6 |
| P-41 | p-41/p-70 (90/10) | 11,000 | 1.6 |
| P-42 | p-41/p-71 (80/20) | 12,000 | 1.6 |
| P-43 | p-30 (100) | 12,000 | 1.6 |
| P-44 | p-36/p-73 (90/10) | 11,500 | 1.6 |
| P-45 | p-37 (100) | 15,000 | 1.6 |
| P-46 | p-22/p-75 (90/10) | 8,500 | 1.6 |
| P-47 | p-8/p-63 (90/10) | 6,500 | 1.6 |
| P-48 | p-15/p-38 (98/2) | 7,000 | 1.6 |
| P-49 | p-2 (100) | 11,000 | 1.5 |
| P-50 | p-31/p-70 (98/2) | 12,000 | 1.6 |
| P-51 | p-55/0-69 (90/10) | 9,000 | 1.6 |
| P-52 | p-56/p-68 (80/20) | 9,000 | 1.4 |
| P-53 | p-6/p-2 (50/50) | 7,000 | 1.6 |
| P-54 | p-57/p-74 (80/20) | 8,000 | 1.6 |
| P-55 | p-57/p-75 (70/30) | 9,000 | 1.6 |
| P-56 | p-57/p-67 (90/10) | 10,000 | 1.6 |
| P-57 | p-48/p-72 (98/2) | 15,000 | 1.6 |
| P-58 | p-48 (100) | 16,000 | 1.6 |
| P-59 | p-16 (100) | 10,000 | 1.4 |
| P-60 | p-19/p-67 (98/2) | 9,000 | 1.6 |
| P-61 | p-39/p-32 (80/20) | 9,000 | 1.5 |
| P-62 | p-19 (100) | 11,000 | 1.6 |
| P-63 | p-8/p-74 (80/20) | 12,000 | 1.4 |
| P-64 | p-49 (100) | 12,000 | 1.6 |
| P-65 | p-47 (100) | 14,500 | 1.4 |
| P-66 | p-53/p-61 (80/20) | 12,500 | 1.6 |
| P-67 | p-4/p-70 (80/20) | 12,000 | 1.6 |
| P-68 | p-4/p-71 (80/20) | 13,500 | 1.6 |
| P-69 | p-31 (100) | 11,500 | 1.6 |
| P-70 | p-37/p-64 (80/20) | 11,000 | 1.6 |
| P-71 | p-50/p-75 (80/20) | 8,000 | 1.6 |
| P-72 | p-51/p-61 (98/2) | 10,000 | 1.6 |
| P-73 | p-51/p-62 (90/10) | 9,000 | 1.6 |
| P-74 | p-25/p-57 (90/10) | 9,000 | 1.6 |
| P-75 | P-5 (100) | 11,000 | 1.6 |
| P-76 | p-45/p-65 (80/20) | 12,000 | 1.6 |
| P-77 | p-46 (100) | 8,000 | 1.6 |
| P-78 | p-43/p-63 (98/2) | 9,000 | 1.4 |
| P-79 | p-9 (100) | 9,000 | 1.6 |
| P-80 | p-9/p-62 (98/2) | 7,000 | 1.5 |

TABLE 2

| | Composition | | |
|---|---|---|---|
| Resin | (Compositional ratio) | Mw | Pd |
| P-81 | p-11/p-61 (90/10) | 8,000 | 1.6 |
| P-82 | p-43/p-60 (90/10) | 9,000 | 1.4 |
| P-83 | p-14/p-56 (80/20) | 11,000 | 1.4 |
| P-84 | p-29/p-63 (98/2) | 12,000 | 1.6 |
| P-85 | p-52/p-56 (90/10) | 12,000 | 1.6 |
| P-86 | p-3 (100) | 12,000 | 1.6 |
| P-87 | p-26 (100) | 15,000 | 1.6 |
| P-88 | p-33 (100) | 10,000 | 1.6 |
| P-89 | p-33/p-73 (90/10) | 9,000 | 1.6 |
| P-90 | p-52 (100) | 9,000 | 1.6 |
| P-91 | p-52/p-57 (50/50) | 11,000 | 1.6 |
| P-92 | p-59 (100) | 12,000 | 1.6 |
| P-93 | p-8/p-63 (98/2) | 10,000 | 1.5 |
| P-94 | p-24 (100) | 9,000 | 1.6 |
| P-95 | p-1/p-65 (98/2) | 9,000 | 1.6 |
| P-96 | p-24/p-70 (50/50) | 11,000 | 1.4 |
| P-97 | p-32 (100) | 12,000 | 1.6 |
| P-98 | p-32/p-69 (90/10) | 12,000 | 1.6 |
| P-99 | p-53C100) | 11,500 | 1.6 |
| P-100 | p-53/p-66 (80/20) | 12,000 | 1.6 |
| P-101 | p-13/p-66 (80/20) | 9,000 | 1.6 |
| P-102 | p-34 (100) | 9,000 | 1.6 |
| P-103 | p-39 (100) | 11,000 | 1.6 |
| P-104 | p-22 (100) | 8,,500 | 1.6 |
| P-105 | p-46/p-66 (90/10) | 11,000 | 1.4 |
| P-106 | p-58 (100) | 12,000 | 1.6 |
| P-107 | p-58/p-68 (98/2) | 9,000 | 1.5 |
| P-108 | p-10/p-68 (90/10) | 7,000 | 1.6 |
| P-109 | p-6 (100) | 8,000 | 1.4 |
| P-110 | p-49 (100) | 8,000 | 1.6 |
| P-111 | p-49/p-63 (90/10) | 7,000 | 1.4 |
| P-112 | p-49/p-60 (98/2) | 12,000 | 1.6 |
| P-113 | p-58/p-75 (80/20) | 15,000 | 1.6 |
| P-114 | p-59/p-66 (90/10) | 10,000 | 1.6 |
| P-115 | p-28/p-62 (80/20) | 9,000 | 1.6 |
| P-116 | p-7 (100) | 14,000 | 1.6 |
| P-117 | p-21/p-72 (80/20) | 7,,500 | 1.6 |
| P-118 | p-2/p-40 (90/10) | 8,000 | 1.6 |
| P-119 | p-35 (100) | 9,000 | 1.6 |
| P-120 | p-49/p-76 (90/10) | 10,000 | 1.6 |
| P-121 | p-49/p-60/p-87 (90/8/2) | 9,000 | 1.6 |
| P-122 | p-59/p-81 (80/20) | 9,000 | 1.6 |
| P-123 | p-58/p-81 (91/9) | 11,000 | 1.6 |
| P-124 | p-8/p-85 (83/17) | 13,000 | 1.6 |
| P-125 | p-22/p-84 (90/10) | 8,000 | 1.6 |
| P-126 | p-2/p-77 (90/10) | 9,000 | 1.4 |
| P-127 | p-25/p-80 (95/5) | 9,000 | 1.6 |
| P-128 | p-2 (100) | 7,000 | 1.5 |
| P-129 | p-31/p-70/p-79 (80/18/2) | 12,000 | 1.6 |
| P-130 | p-55/p-69/p-63 (90/5/5) | 11,500 | 1.6 |
| P-131 | p-56/p-83 (80/20) | 15,000 | 1.6 |
| P-132 | p-37/p-82 (81/19) | 8,500 | 1.6 |
| P-133 | p-50/p-75/p-76 (80/15/5) | 6,500 | 1.5 |
| P-134 | p-10/p-86 (94/6) | 7,000 | 1.6 |
| P-135 | p-17/p-79 (89/11) | 11,000 | 1.6 |
| P-136 | p-25/p-87 (92/8) | 8,500 | 1.4 |
| P-137 | p-54/p-82 (82/18) | 9,000 | 1.6 |
| P-138 | p-42/p-76 (80/20) | 10,000 | 1.6 |
| P-139 | p-16/p-62/p-77 (88/10/2) | 11,000 | 1.6 |
| P-140 | p-23/p-85 (90/10) | 12,000 | 1.6 |
| P-141 | p-20/p-80 (90/10) | 11,000 | 1.6 |
| P-142 | p-32/p-82 (89/11) | 9,000 | 1.6 |
| P-143 | p-5/p-84 (83/17) | 9,000 | 1.6 |
| P-144 | p-3/p-86 (90/10) | 11,000 | 1.5 |
| P-145 | p-44/p-71/p-80 (91/7/2) | 10,000 | 1.6 |
| P-146 | p-1/p-84 (85/15) | 9,000 | 1.6 |
| P-147 | p-33/p-76 (70/30) | 9,000 | 1.4 |
| P-148 | p-30/p-79 (82/18) | 11,000 | 1.6 |
| P-149 | p-57/p-78 (78/22) | 9,000 | 1.6 |
| P-150 | p-57/p-84 (88/12) | 9,000 | 1.6 |
| P-151 | p-19/p-83 (88/12) | 11,000 | 1.6 |
| P-152 | p-55/p-77/p-78 (90/5/5) | 8,500 | 1.6 |
| P-153 | p-38/p-87 (80/20) | 12,000 | 1.6 |
| P-154 | p-6/p-7/p-84 (50/40/10) | 12,000 | 1.4 |
| P-155 | p-41/p-83 (96/4) | 15,000 | 1.6 |
| P-156 | p-40/p-78 (80/20) | 9,000 | 1.5 |
| P-157 | p-21/p-82 (86/14) | 14,000 | 1.6 |
| P-158 | p-2/p-81 (89/11) | 8,500 | 1.6 |
| P-159 | p-9/p-88 (70/30) | 11,000 | 1.6 |

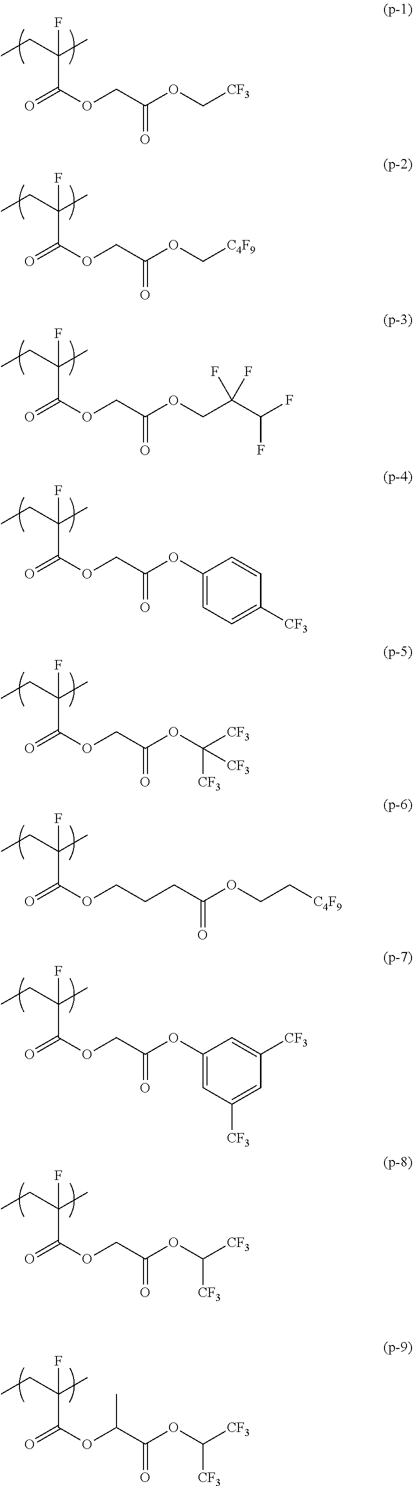

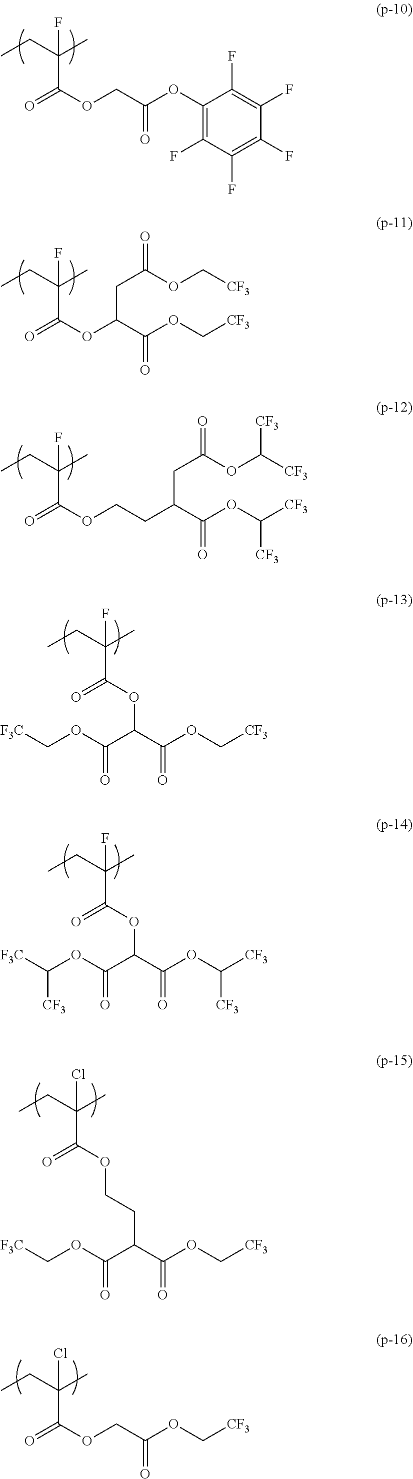

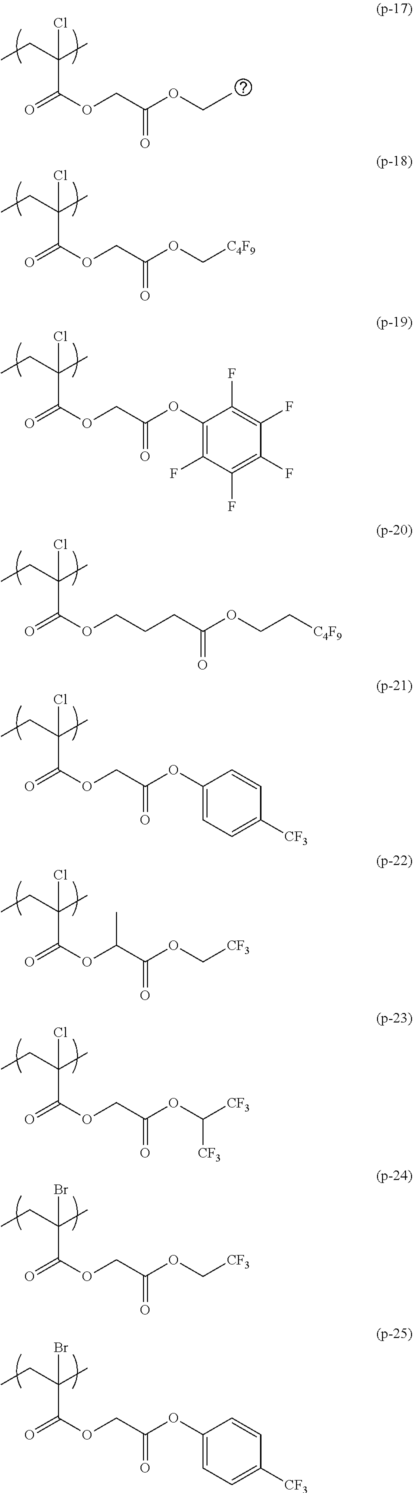

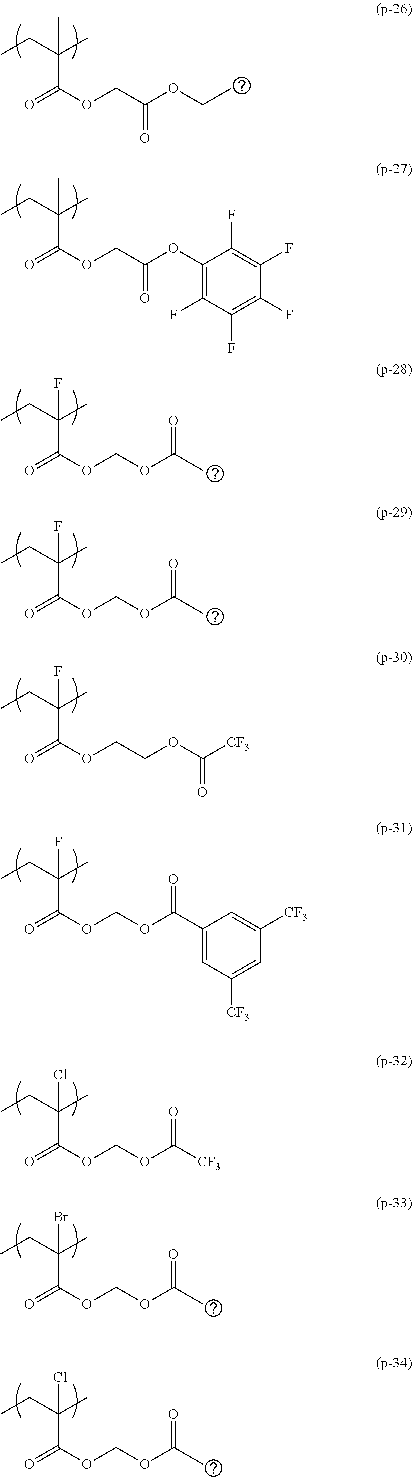

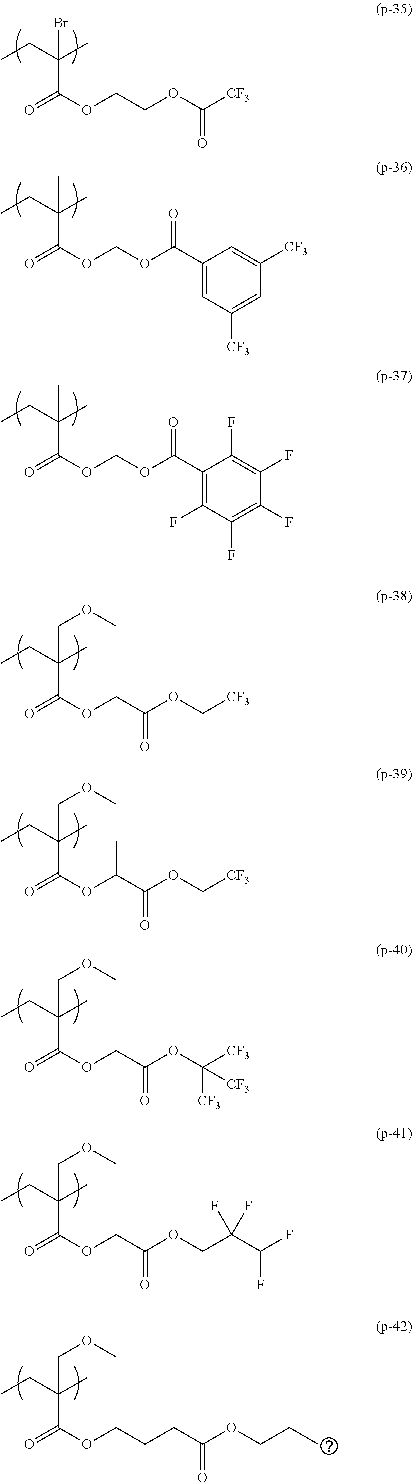

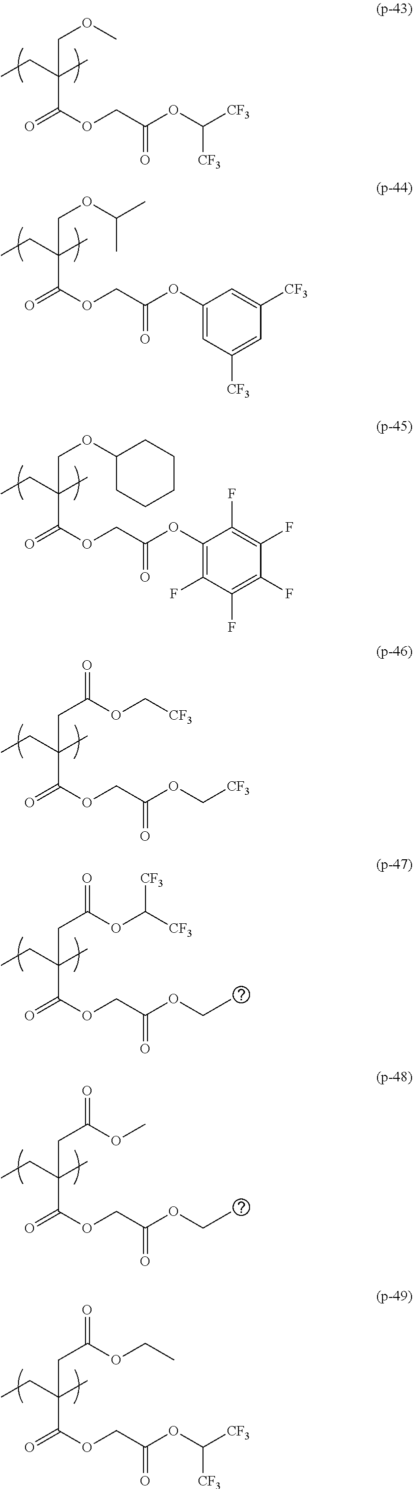

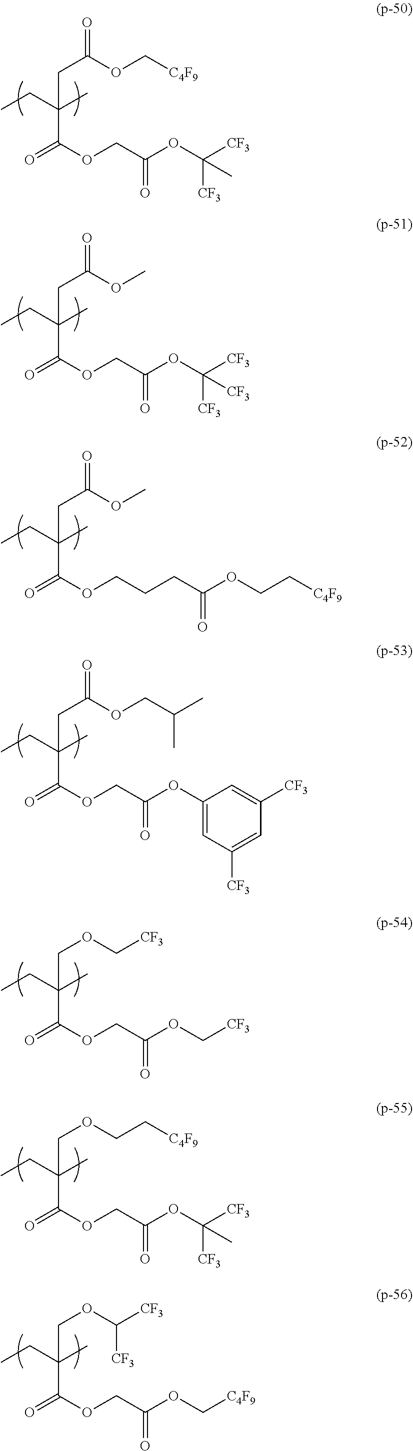

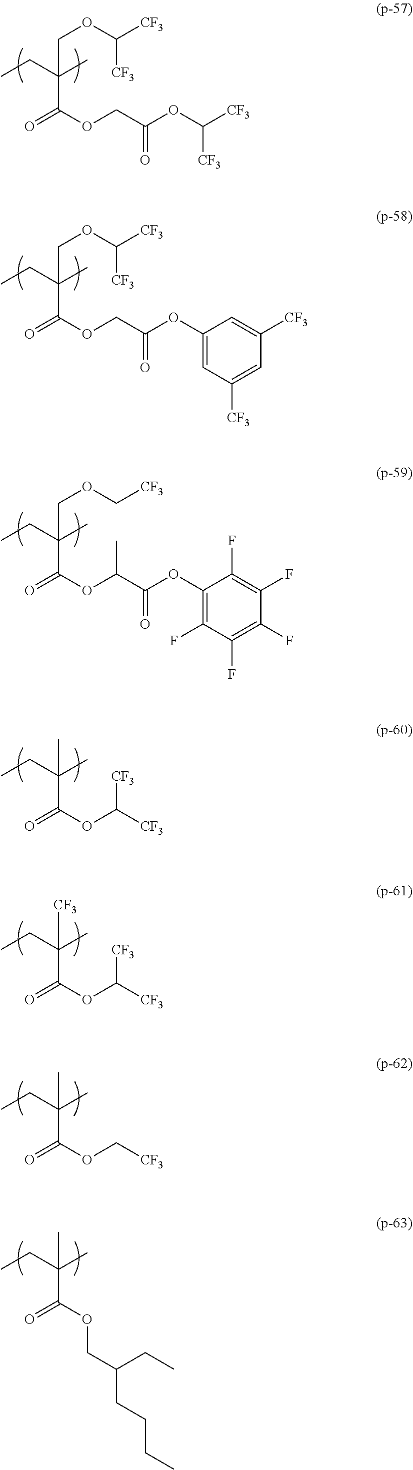

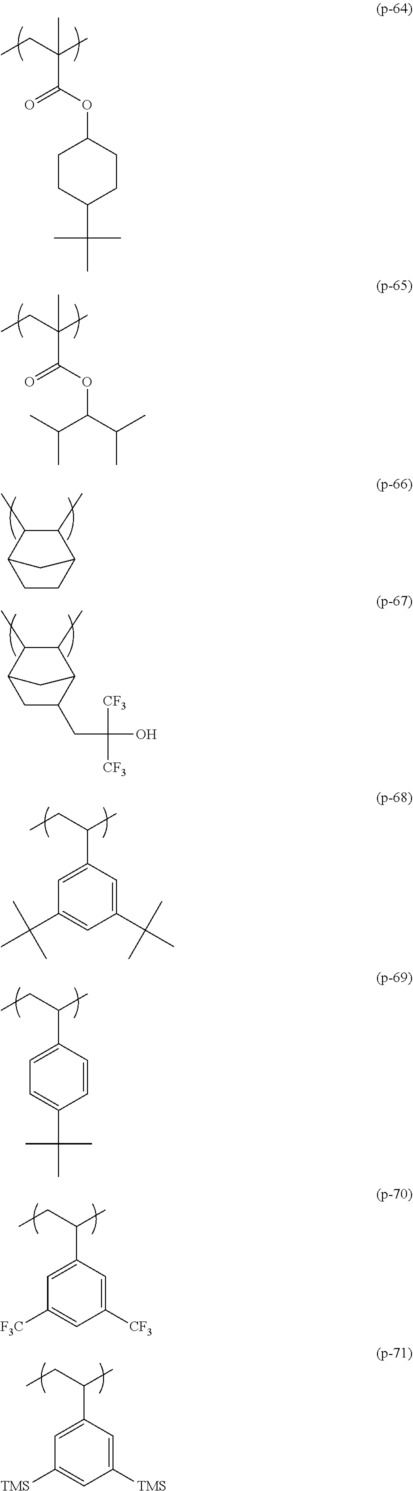

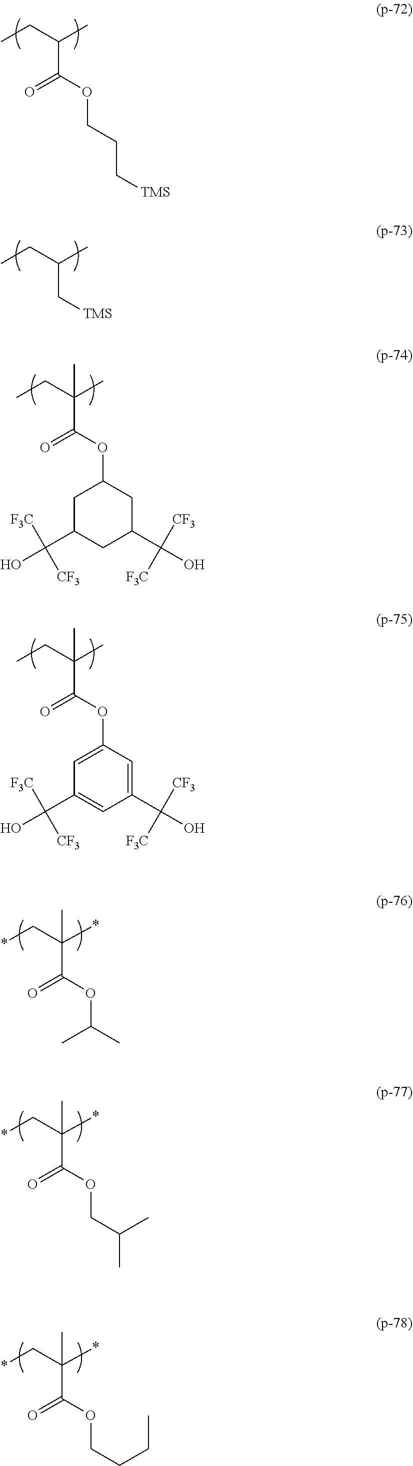

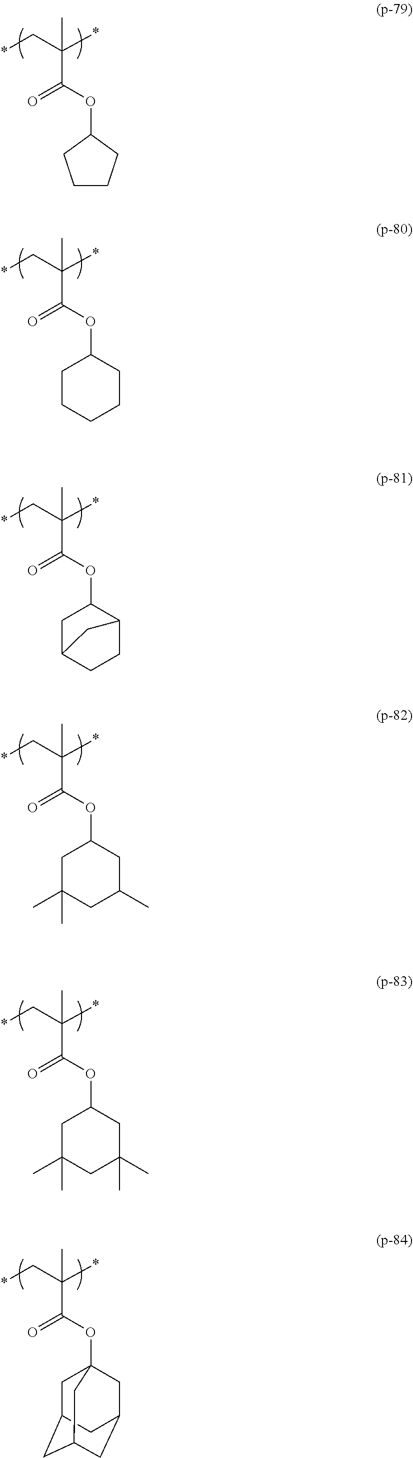

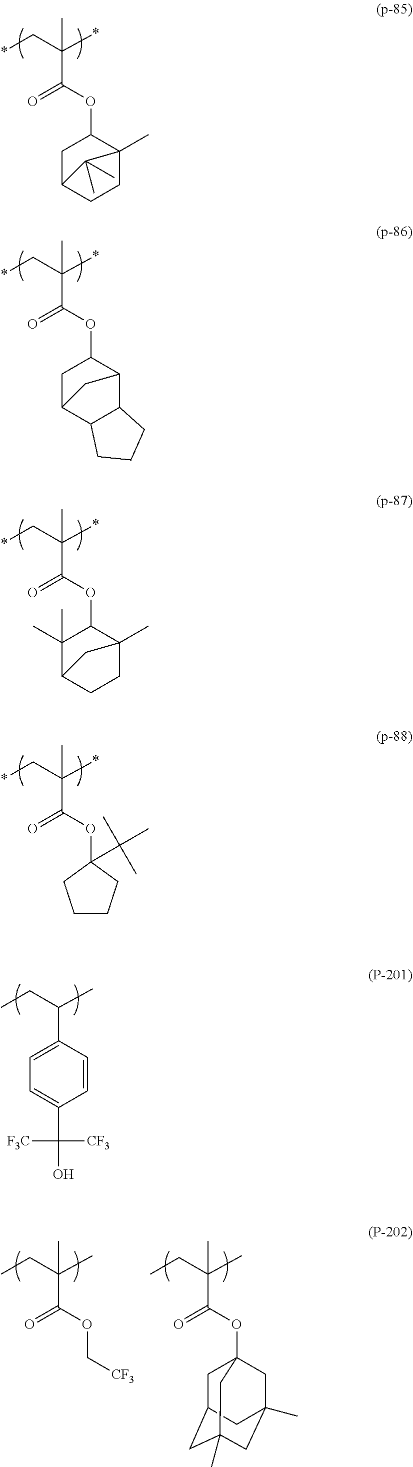

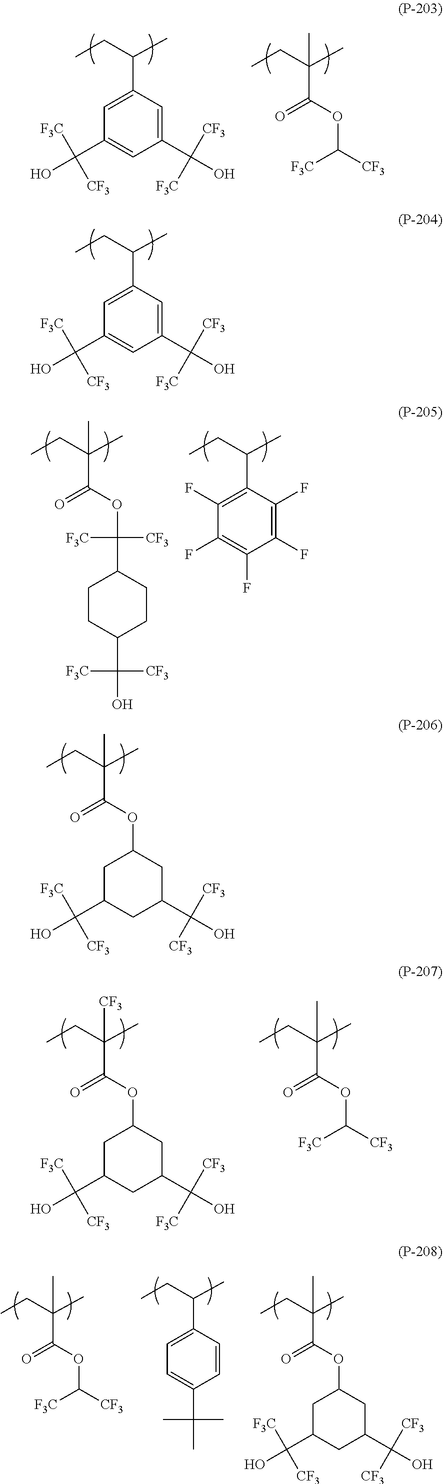

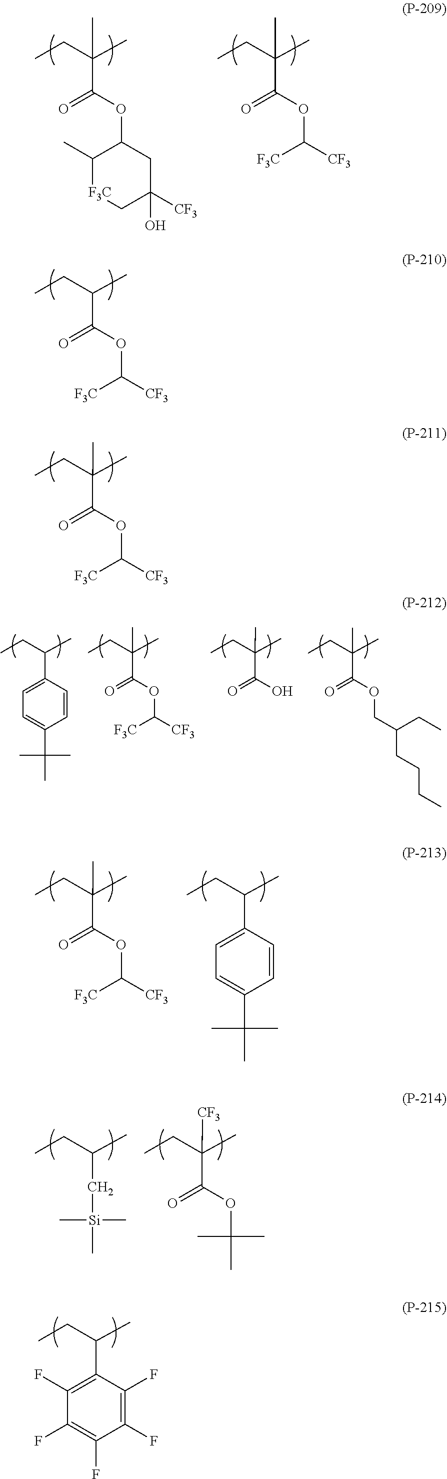

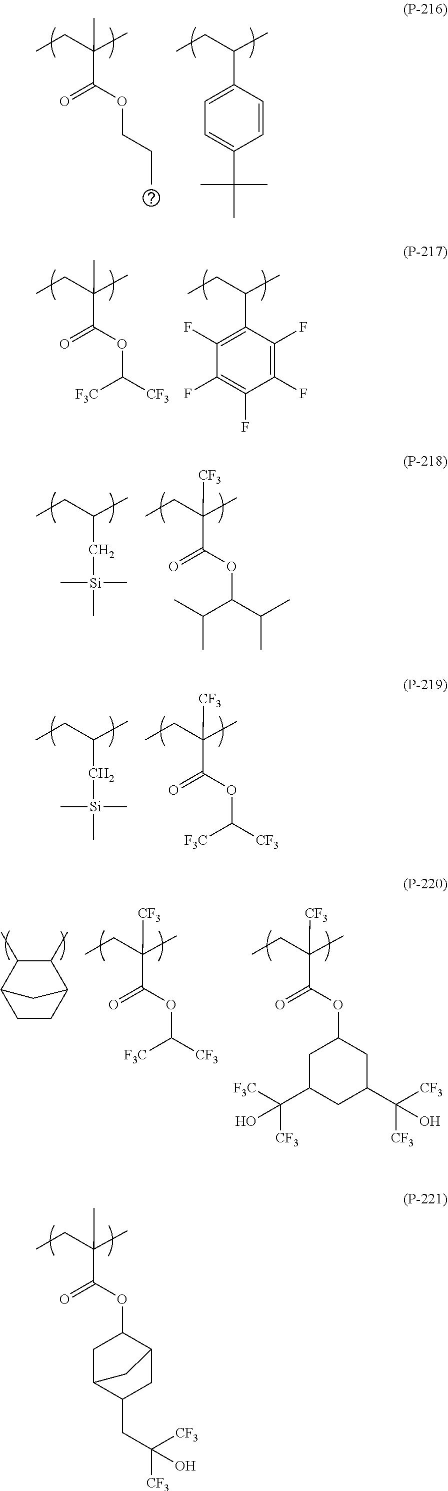

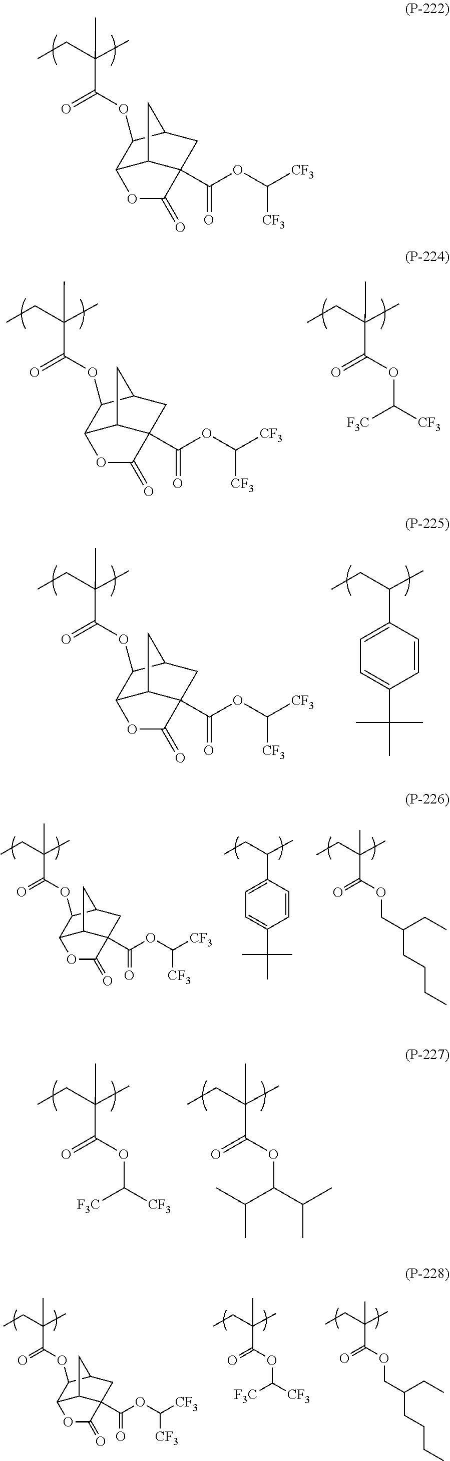

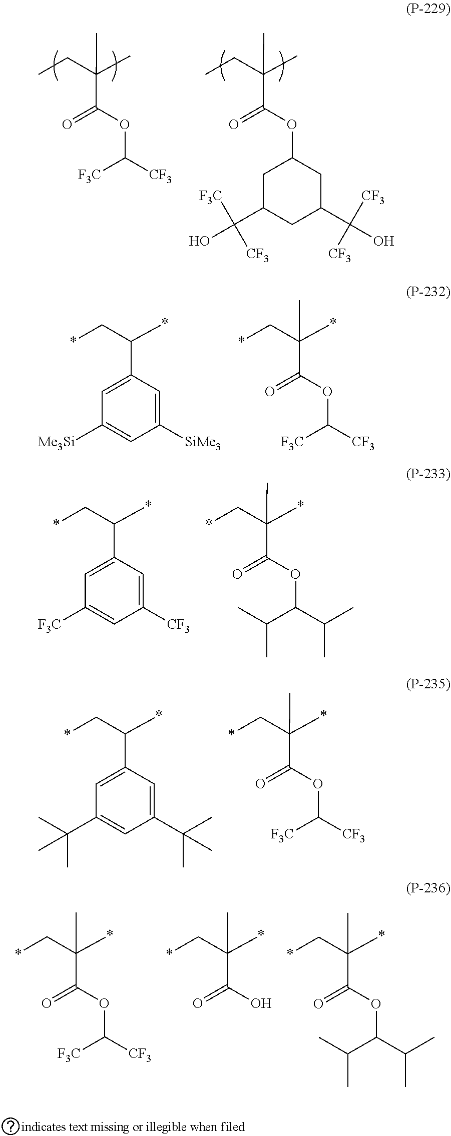

TABLE 3

| | Composition | | |
|---|---|---|---|
| Resin | (Compositional ratio) | Mw | Pd |
| P-201 | 100 | 9,000 | 1.6 |
| P-202 | 70/30 | 11,000 | 1.6 |
| P-203 | 60/40 | 12,000 | 1.4 |
| P-204 | 100 | 12,000 | 1.6 |
| P-205 | 90/10 | 11,500 | 1.6 |
| P-206 | 100 | 13,500 | 1.6 |
| P-207 | 40/60 | 11,500 | 1.6 |
| P-208 | 60/10/30 | 11,000 | 1.5 |
| P-209 | 40/60 | 8,000 | 1.6 |
| P-210 | 100 | 10,000 | 1.5 |
| P-211 | 100 | 9,000 | 1.6 |
| P-212 | 56/40/2/2 | 9,000 | 1.4 |
| P-213 | 50/50 | 9,000 | 1.6 |
| P-214 | 50/50 | 7,000 | 1.5 |
| P-215 | 100 | 8,000 | 1.6 |
| P-216 | 30/70 | 8,000 | 1.4 |
| P-217 | 80/20 | 7,000 | 1.4 |
| P-218 | 50/50 | 12,000 | 1.6 |
| P-219 | 50/50 | 15,000 | 1.6 |
| P-220 | 50/25/25 | 10,000 | 1.5 |
| P-221 | 100 | 9,000 | 1.6 |
| P-222 | 100 | 12,000 | 1.6 |
| P-224 | 60/40 | 10,000 | 1.6 |
| P-225 | 40/60 | 8,500 | 1.6 |
| P-226 | 60/38/2 | 9,000 | 1.4 |
| P-227 | 95/5 | 11,000 | 1.6 |
| P-228 | 60/38/2 | 16,000 | 1.6 |
| P-229 | 90/10 | 14,500 | 1.6 |
| P-232 | 80/20 | 13,500 | 1.4 |
| P-233 | 60/40 | 9,000 | 1.6 |
| P-235 | 60/40 | 9,000 | 1.6 |
| P-236 | 91/2/7 | 13,500 | 1.4 |

The hydrophobic resins (E) may be used singly or in combination of two or more kinds thereof.

It is preferable to use a mixture of two or more kinds of hydrophobic resins (E) having different levels of surface energy from the viewpoint of satisfying both the immersion liquid tracking properties and the development characteristics upon liquid immersion exposure.

The content of the hydrophobic resin (E) in the composition is preferably 0.01% to 10% by mass, and more preferably 0.05% to 8% by mass, with respect to the total solid content in the photosensitive resin composition according to the present disclosure.

In a case where the composition according to the present disclosure includes the resin (A) as a resin and further includes the resin (E), the content ratio (mass ratio) of the resin (A) to the resin (E) is preferably resin (A):resin (E)=1:0.001 to 1:0.2, and more preferably 1:0.01 to 1:0.1.

<Low-Molecular-Weight Ester Compound>

The photosensitive resin composition according to the present disclosure preferably contains a low-molecular-weight ester compound.

The low-molecular-weight ester compound is a compound which has alkali decomposability and has a molecular weight of less than 1,500.

Furthermore, a compound corresponding to a photoacid generator which will be described later is construed not to correspond to the low-molecular-weight ester compound.

It is preferable that the low-molecular-weight ester compound according to the present disclosure has no acid-decomposable group.

In addition, it is preferable that the low-molecular-weight ester compound according to the present disclosure does not decompose upon exposure with light.

[Alkali Decomposability]

The low-molecular-weight ester compound used in the present disclosure has alkali decomposability.

In the present disclosure, the expression, alkali decomposability, means a property of causing a decomposition reaction by the action of an aqueous alkali solution.

An expression, “having alkali decomposability”, means that in a case where 100 mg of an ester compound is added to a mixed liquid of 2 mL of a buffer solution at pH 10 and 8 of tetrahydrofuran (THF), the mixture is left to stand at 40° C., and thus, 30% by mole or more of the total amount of the ester bonds contained in the ester compound is hydrolyzed after 10 minutes. In addition, the decomposition rate can be calculated from a ratio of a raw material to a decomposition product, determined by nuclear magnetic resonance (NMR) analysis.

[Molecular Weight]

The molecular weight of the low-molecular-weight ester compound is less than 1,500, preferably 1,000 or less, and more preferably 600 or less.

A lower limit of the molecular weight is not particularly limited, but is preferably 50 or more, more preferably 150 or more, still more preferably 200 or more, and particularly preferably 300 or more.

The molecular weight of the low-molecular-weight ester compound is measured by electrospray ion mass spectrometry (ESI-MS).

[Ester Bond]

Examples of the ester bond in the low-molecular-weight ester compound used in the present disclosure include a carboxylic acid ester bond, a sulfonic acid ester bond, and a phosphoric acid ester bond, and the carboxylic acid ester bond is preferable.

The number of the ester bonds (carboxylic acid ester bonds) in the low-molecular-weight ester compound is preferably from 1 to 10, more preferably from 1 to 4, and still more preferably 1 or 2.

[Alkyl Group or Alkylene Group]

From the viewpoint of improving the pattern shape, it is preferable that the low-molecular-weight ester compound used in the present disclosure includes an alkyl group having 5 or more carbon atoms or an alkylene group having 4 or more carbon atoms, and it is more preferable that the low-molecular-weight ester compound used in the present disclosure includes the alkyl group having 5 or more carbon atoms.

From the viewpoint of improving the pattern shape, as the alkyl group having 5 or more carbon atoms, an alkyl group having 8 or more carbon atoms is preferable, and an alkyl group having 10 or more carbon atoms is more preferable.

An upper limit of the number of carbon atoms is not particularly limited, and is preferably 40 or less, and more preferably 30 or less.

The alkyl group having 5 or more carbon atoms may be linear, branched, or cyclic, and may also be a group formed by combination thereof.

The alkyl group having 5 or more carbon atoms may have a substituent, but the alkyl group having a fluorine atom as a substituent is construed to correspond to a halogenated alkyl group which will be described later.

The alkyl group having 5 or more carbon atoms is preferably directly bonded to the bonding site on the carbon atom side of the ester bond.

From the viewpoint of improving the pattern shape, as the alkylene group having 4 or more carbon atoms, an alkylene group having 6 or more carbon atoms is preferable, and an alkylene group having 10 or more carbon atoms is more preferable.

An upper limit of the number of carbon atoms is not particularly limited, and is preferably 40 or less, and more preferably 30 or less.

The alkylene group having 4 or more carbon atoms may be linear, branched, or cyclic, and may also be a group formed by combination thereof.

It is preferable that at least one of the two bonding sites of the alkylene group is preferably directly bonded to the bonding site of the carbon atom side of the ester bond, and it is more preferable that both the two bonding sites are directly bonded to the bonding site of the carbon atom side of the ester bond.

[Electron-Withdrawing Group]

From the viewpoints of improving the pattern shape, improving the tolerance of DOF, and suppressing the development defects, it is preferable that the low-molecular-weight ester compound has at least one or more electron-withdrawing groups. The number of the electron-withdrawing groups is not particularly limited, and is preferably 1 to 5, and more preferably 1 to 4.

Examples of the electron-withdrawing group include known electron-withdrawing groups, and the electron-withdrawing group is preferably a halogenated alkyl group, a halogen atom, a cyano group, a nitro group, or a group represented by —COO-Rb (Rb represents an alkyl group), and more preferably the halogenated alkyl group.

In addition, examples of the halogen atom in the halogenated alkyl group include a fluorine atom, a chlorine atom, a bromine atom, and an iodine atom.

Among those, the low-molecular-weight ester compound used in the present disclosure preferably includes a fluorinated alkyl group.

[Halogenated Alkyl Group]

The low-molecular-weight ester compound used in the present disclosure preferably includes a halogenated alkyl group, and more preferably includes a fluorinated alkyl group.

The halogenated alkyl group may be linear, branched, or cyclic, and may also be a group formed by combination thereof.

The halogenated alkyl group may be the group in which at least one of the hydrogen atoms in the alkyl group is substituted with a halogen atom, but it is preferably the group in which all of the hydrogen atoms in the alkyl group are substituted with fluorine atoms.

The number of carbon atoms in the halogenated alkyl group is preferably from 1 to 10, more preferably from 1 to 4, more preferably 1 or 2, and still more preferably 1.

That is, as the halogenated alkyl group, a trifluoromethyl group is particularly preferable.

The halogenated alkyl group may be present at any site in the low-molecular-weight ester compound, but it is preferably directly bonded to the carbon atom directly bonded to the bonding site of the oxygen atom side of the ester bond. Further, the number of the halogenated alkyl groups bonded to the carbon atoms is preferably 1 or 2, and more preferably 2.

[Chained Ester Compound]

From the viewpoint of improving the shape of the pattern, it is preferable that the low-molecular-weight ester compound is a chained ester compound.

In the present disclosure, the chained ester compound refers to a compound in which the ester compound is not included in the ring structure.

In a case where the low-molecular-weight ester compound has a plurality of the ester bonds, it is preferably an ester compound in which at least one of the ester bonds is not included in the ring structure, and more preferably an ester compound in which all of the ester bonds are not included in the ring structure.

[ClogP Value]

The ClogP value of the low-molecular-weight ester compound is not particularly limited, and is preferably 1 to 12, and more preferably 3 to 11.

The ClogP value is a calculated value as computed from LogP expressed in a common logarithm logP of a partition coefficient P in water-n-octanol, and has been used as an index indicating a hydrophilicity/hydrophobicity degree of a substance. The ClogP of the low-molecular-weight ester compound can be calculated by using, for example, ChemDraw Ultra 8.0, software from Cambridge Soft Corporation.

[Partial Structure Represented by Formula A]

The ester compound preferably has a partial structure represented by Formula A. * represents a bonding position. An ester compound having the following partial structure has alkali decomposability.

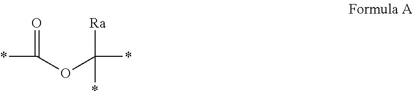

In Formula A, Ra represents an electron-withdrawing group. Suitable aspects of the electron-withdrawing group are as described above.

The low-molecular-weight ester compound used in the present disclosure is preferably a compound represented by Formula B.

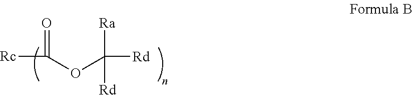

In Formula B, Ra represents an electron-withdrawing group, Rc represents an n-valent hydrocarbon group, Rd's each independently represent a hydrogen atom or a substituent, and n represents an integer of 1 to 3. In a case where n is 2 or more, Ra's may be the same as or different from each other.

In Formula B, Ra represents an electron-withdrawing group. Suitable aspects of the electron-withdrawing group are as described above.

Rc represents an n-valent hydrocarbon group. The number of carbon atoms in the hydrocarbon group is not particularly limited, and from the viewpoint that the effects according to the present disclosure are excellent, the number of carbon atoms is preferably 2 to 25, and more preferably 3 to 20.

The hydrocarbon group may be chained or cyclic. Among those, from the viewpoint that the effects according to the present disclosure are more excellent, a chained hydrocarbon group is preferable. The chained hydrocarbon group may be linear or branched.

In addition, Rc is preferably the above-mentioned alkyl group having 5 or more carbon atoms or the above-mentioned alkylene group having 4 or more carbon atoms, and more preferably the above-mentioned alkyl group having 5 or more carbon atoms.

Rd's each independently represent a hydrogen atom or a substituent.

Examples of the substituent include a halogen atom such as a fluorine atom, a chlorine atom, a bromine atom, and an iodine atom; an alkoxy group such as a methoxy group, an ethoxy group, and a tert-butoxy group; an aryloxy group such as a phenoxy group and a p-tolyloxy group; an alkoxycarbonyl group such as a methoxycarbonyl group, a butoxycarbonyl group, and a phenoxycarbonyl group; an acyloxy group such as an acetoxy group, a propionyloxy group, and a benzoyloxy group; an acyl group such as an acetyl group, a benzoyl group, an isobutyryl group, an acryloyl group, a methacryloyl group, and a methoxalyl group; an alkylsulfanyl group such as a methyisulfanyl group and a tert-butylsulfanyl group; an arylsulfanyl group such as a phenylsulfanyl group and a p-tolylsulfanyl group; an alkyl group; a cycloalkyl group; an aryl group; a heteroaryl group; a hydroxyl group; a carboxyl group; a formyl group; a sulfo group; a cyano group; an alkylaminocarbonyl group; an arylaminocarbonyl group; a sulfonamido group; a silyl group; an amino group; a monoalkylamino group; a dialkylamino group; an arylamino group; and a combination thereof.

Among those, from the viewpoint that the effects according to the present disclosure are more excellent, it is preferable that at least one of Rd's is an electron-withdrawing group. Suitable aspects of the electron-withdrawing group are as described above.

n represents an integer of 1 to 3. n is preferably 1 or 2.

Specific examples of the low-molecular-weight ester compound used in the present disclosure are shown below, but are not limited thereto.

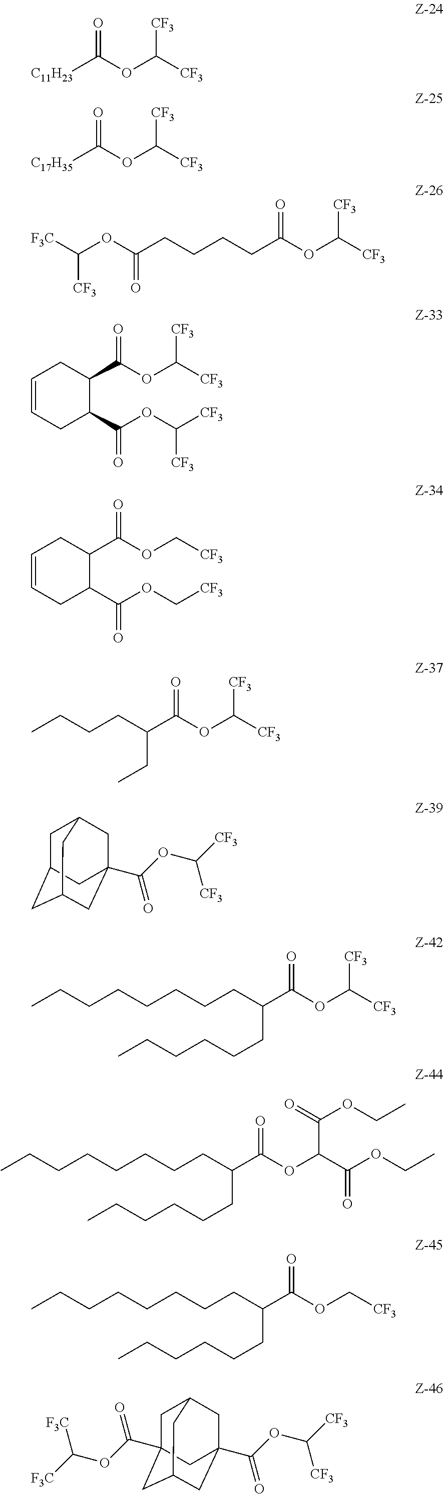

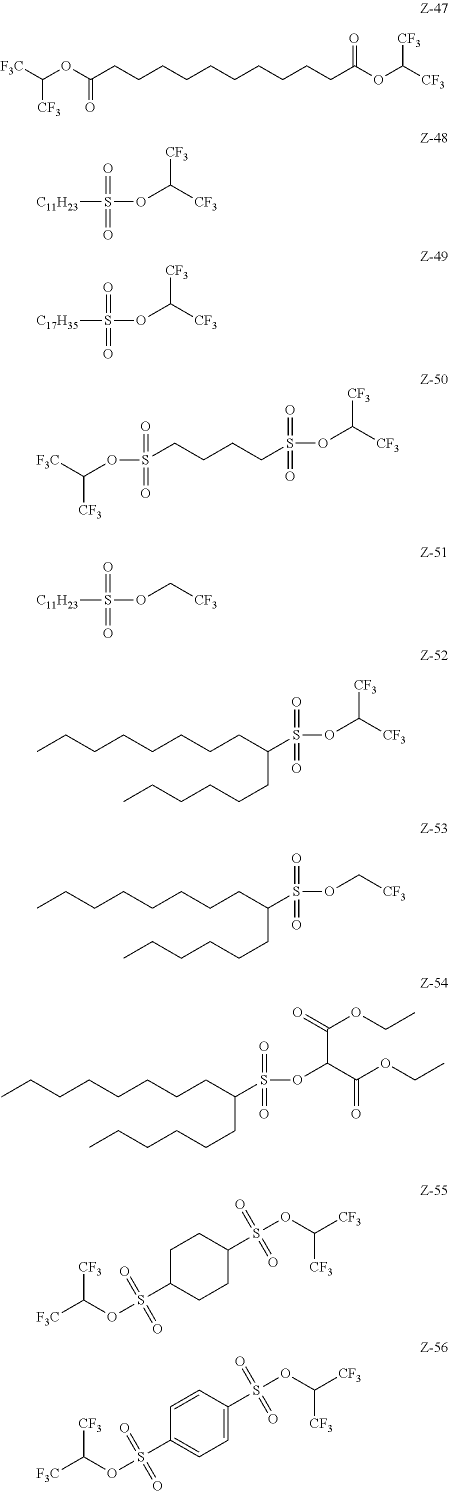

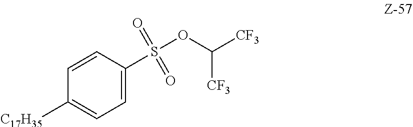

In the photosensitive resin composition according to the present disclosure, the low-molecular-weight ester compounds may be contained singly or may be used in combination of two or more kinds thereof.

The content of the low-molecular-weight ester compound is preferably from 0.1% by mass to 6% by mass, more preferably from 1.0% by mass to 5.0% by mass, and particularly preferably from 1.5% by mass to 4.0% by mass, with respect to the total solid content of the composition.

<Other Acid Diffusion Control Agents>

The photosensitive resin composition according to the present disclosure may include an acid diffusion control agent other than the compound represented by Formula D (“other acid diffusion control agents”). Such other acid diffusion control agents act as a quencher that suppresses a reaction of the acid-decomposable resin in the unexposed area by excessive generated acids by trapping the acids generated from a photoacid generator or the like upon exposure. For example, a basic compound (DA), a basic compound (DB) whose basicity is reduced or lost upon irradiation with actinic rays or radiation, an onium salt (DC) which becomes a relatively weak acid with respect to a photoacid generator, a low-molecular-weight compound (DD) which has a nitrogen atom and a group that leaves by the action of an acid, an onium salt compound (DE) having a nitrogen atom in a cationic moiety, or the like can be used as the acid diffusion control agent. In the composition according to the present disclosure, a known acid diffusion control agent can be appropriately used. For example, the known compounds disclosed in paragraphs 0627 to 0664 of US2016/0070167A1, paragraphs 0095 to 0187 of US2015/0004544A1, paragraphs 0403 to 0423 of US2016/0237190A1, and paragraphs 0259 to 0328 of US2016/0274458A1 can be suitably used as the acid diffusion control agent (D).

[Basic Compound (DA)]

Preferred examples of the basic compound (DA) include compounds having structures represented by Formulae A to F.

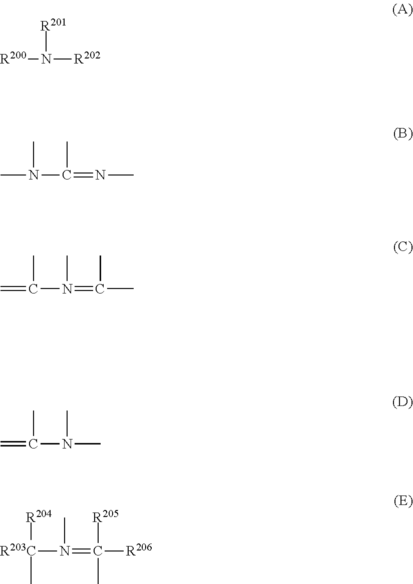

In Formula A and Formula F,

R²⁰⁰, R²⁰¹, and R²⁰² may be the same as or different from each other and each independently represent a hydrogen atom, an alkyl group (preferably having 1 to 20 carbon atoms), a cycloalkyl group (preferably having 3 to 20 carbon atoms), or an aryl group (having 6 to 20 carbon atoms). R²⁰¹ and R²⁰² may be bonded to each other to form a ring.

R²⁰³, R²⁰⁴, R²⁰⁵, and R²⁰⁶ may be the same as or different from each other and each independently represent an alkyl group having 1 to 20 carbon atoms.

The alkyl group in each of Formulae A and F may have a substituent or may be unsubstituted.

With regard to the alkyl group, the alkyl group having a substituent is preferably an aminoalkyl group having 1 to 20 carbon atoms, a hydroxyalkyl group having 1 to 20 carbon atoms, or a cyanoalkyl group having 1 to 20 carbon atoms.

The alkyl groups in each of Formulae A and F are more preferably unsubstituted.

As the basic compound (DA), guanidine, aminopyrrolidine, pyrazole, pyrazoline, piperazine, aminomorpholine, aminoalkylmorpholine, piperidine, or the like is preferable; and a compound having an imidazole structure, a diazabicyclo structure, an onium hydroxide structure, an onium carboxylate structure, a trialkylamine structure, an aniline structure, or a pyridine structure, an alkylamine derivative having a hydroxyl group and/or an ether bond, and an aniline derivative having a hydroxyl group and/or an ether bond, or the like is more preferable.

[Basic Compound (DB) whose Basicity is Reduced or Lost Upon Irradiation with Actinic Rays or Radiation]

The basic compound (DB) whose basicity is reduced or lost upon irradiation with actinic rays or radiation (hereinafter also referred to as a “compound (DB)”) is a compound which has a proton-accepting functional group, and decomposes under irradiation with actinic rays or radiation to exhibit deterioration in proton-accepting properties, no proton-accepting properties, or a change from the proton-accepting properties to acidic properties.

The proton-accepting functional group refers to a functional group having a group or an electron which is capable of electrostatically interacting with a proton, and for example, means a functional group with a macrocyclic structure, such as a cyclic polyether, or a functional group having a nitrogen atom having an unshared electron pair not contributing to π-conjugation. The nitrogen atom having an unshared electron pair not contributing to π-conjugation is, for example, a nitrogen atom having a partial structure represented by the following formula.

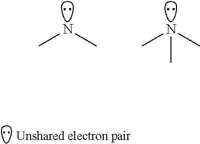

Preferred examples of the partial structure of the proton-accepting functional group include a crown ether structure, an azacrown ether structure, primary to tertiary amine structures, a pyridine structure, an imidazole structure, and a pyrazine structure.

The compound (DB) decomposes upon irradiation with actinic rays or radiation to generate a compound exhibiting deterioration in proton-accepting properties, no proton-accepting properties, or a change from the proton-accepting properties to acidic properties. Here, exhibiting deterioration in proton-accepting properties, no proton-accepting properties, or a change from the proton-accepting properties to acidic properties means a change of proton-accepting properties due to the proton being added to the proton-accepting functional group, and specifically a decrease in the equilibrium constant at chemical equilibrium in a case where a proton adduct is generated from the compound (DB) having the proton-accepting functional group and the proton.

The proton-accepting properties can be confirmed by performing pH measurement.

The acid dissociation constant pKa of a compound generated by the decomposition of the compound (DB) upon irradiation with actinic rays or radiation preferably satisfies pKa<−1, more preferably −13<pKa<−1, and still more preferably −13<pKa<−3.

The acid dissociation constant pKa indicates an acid dissociation constant pKa in an aqueous solution, and is defined, for example, in Chemical Handbook (II) (Revised 4^{th} Edition, 1993, compiled by the Chemical Society of Japan, Maruzen Company, Ltd.). A lower value of the acid dissociation constant pKa indicates higher acid strength. Specifically, the acid dissociation constant pKa in an aqueous solution may be measured by using an infinite-dilution aqueous solution and measuring the acid dissociation constant at 25° C. Alternatively, the acid dissociation constant pKa can also be determined using the following software package 1, by computation from a value based on a Hammett substituent constant and database of publicly known literature values. Any of the values of a pKa described in the present specification represent values determined by calculation using the software package.

Software package 1: Advanced Chemistry Development (ACD/Labs) Software V 8.14 for Solaris (1994-2007 ACD/Labs).

[Onium Salt (DC) which Becomes Relatively Weak Acid with Respect to Photoacid Generator]

In the photosensitive resin composition according to the present disclosure, an onium salt (DC) which becomes a relatively weak acid with respect to the photoacid generator can be used as such another acid diffusion control agent.

In a case of mixing a photoacid generator and an onium salt capable of generating an acid which is a relatively weak acid with respect to an acid generated from the photoacid generator, and then using the mixture, in a case where the acid generated from the photoacid generator upon irradiation with actinic rays or radiation collides with an onium salt having an unreacted weak acid anion, a weak acid is jetted by salt exchange, thereby generating an onium salt having a strong acid anion. In this process, since the strong acid is exchanged with a weak acid having a lower catalytic ability, the acid is deactivated in appearance, and it is thus possible to perform the control of acid diffusion.

From the viewpoints of the tolerance of a depth of focus and the pattern linearity, it is preferable that the photosensitive resin composition according to the present disclosure further includes at least one compound selected from the group consisting of compounds represented by Formula d1-1 to Formula d1-3.

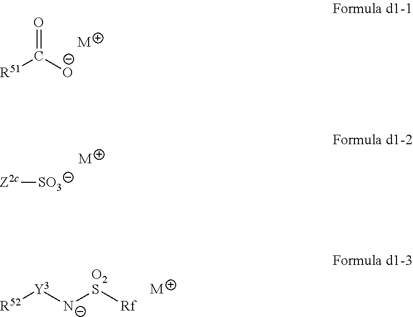

In Formula d1-1 to Formula d1-3, R⁵¹ represents a hydrocarbon group which may have a substituent, Z^{2c} represents a hydrocarbon group having 1 to 30 carbon atoms, which may have a substituent, in which a fluorine atom is not bonded to the carbon atom adjacent to the S atom, R⁵² represents an organic group, Y³ represents a linear, branched, or cyclic alkylene group or an arylene group, Rf represents a hydrocarbon group including a fluorine atom, and M⁺'s each independently represent an ammonium cation, a sulfonium cation, or an iodonium cation.

Preferred examples of the sulfonium cation or the iodoniurn cation represented by M⁺ include the sulfonium cations exemplified for Formula ZI and the iodonium cations exemplified for Formula ZII.

The onium salt (DC) which becomes a relatively weak acid with respect to the photoacid generator may be a compound (hereinafter also referred to as a “compound (DCA)”) having a cationic moiety and an anionic moiety in the same molecule, in which the cationic moiety and the anionic moiety are linked to each other through a covalent bond.

As the compound (DCA), a compound represented by any one of Formulae (C-1), . . . , or (C-3) is preferable.

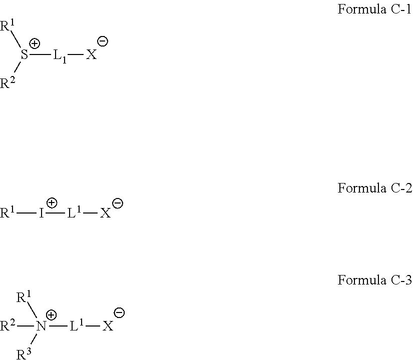

In Formulae C-1 to C-3, R¹, R², and R³ each independently represent a substituent having 1 or more carbon atoms.

L¹ represents a divalent linking group that inks a cationic moiety with an anionic moiety, or a single bond.

—X⁻ represents an anionic moiety selected from —COO⁻, —SO₃⁻, —SO₂⁻, and —N⁻—R⁴. R⁴ represents a monovalent substituent having at least one of a carbonyl group: —C(═O)—, a sulfonyl group: —S(═O)₂—, or a sulfinyl group: −S(═O)— at a site for linking to an adjacent N atom.

R¹, R², R³ R⁴, and L¹ may be bonded to one another to form a ring structure. Further, in Formula C-3, two of R¹ to R³ may be combined to represent a divalent substituent or R¹ to R³ may be bonded to an N atom through a double bond.

Examples of the substituent having 1 or more carbon atoms in each of R¹ to R³ include an alkyl group, a cycloalkyl group, an aryl group, an alkyloxycarbonyl group, a cycloalkyloxycarbonyl group, an aryloxycarbonyl group, an alkylaminocarbonyl group, a cycloalkylaminocarhonyl group, and an arylaminocarbonyl group. The substituent is preferably an alkyl group, a cycloalkyl group, or an aryl group.

Examples of L¹ as the divalent linking group include a linear or branched alkylene group, a cycloalkylene group, an arylene group, a carbonyl group, an ether bond, ester bond, amide bond, a urethane bond, a urea bond, and a group formed by a combination of two or more kinds of these groups. L¹ is preferably an alkylene group, an arylene group, an ether bond, ester bond, and a group formed by a combination of two or more kinds of these groups.

[Low-Molecular-Weight Compound (DD) Having Nitrogen Atom and Group that Leaves by Action of Acid]

The low-molecular-weight compound (DD) (hereinafter referred to as a “compound (DD)”) having a nitrogen atom and a group that leaves by the action of an acid is preferably an amine derivative having a group that leaves by the action of an acid on a nitrogen atom.

As the group that leaves by the action of an acid, an acetal group, a carbonate group, a carbamate group, a tertiary ester group, a tertiary hydroxyl group, or a hemiaminal ether group is preferable, and the carbamate group or the hemiaminal ether group is more preferable.

The molecular weight of the compound (DD) is preferably 100 to 1,000, more preferably 100 to 700, and still more preferably 100 to 500.

The compound (DD) may have a carbamate group having a protecting group on a nitrogen atom. The protecting group constituting the carbamate group is represented by Formula d-1.

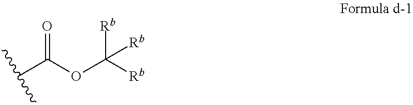

In Formula d-1,

R^{b}'s each independently represent a hydrogen atom, an alkyl group (preferably having 1 to 10 carbon atoms), a cycloalkyl group (preferably having 3 to 30 carbon atoms), an aryl group (preferably having 3 to 30 carbon atoms), an aralkyl group (preferably having 1 to 10 carbon atoms), or an alkoxyalkyl group (preferably having 1 to 10 carbon atoms). R^{b}'s may be linked to each other to form a ring.

The alkyl group, the cycloalkyl group, the aryl group, and the aralkyl group represented by R^{b} may be each independently substituted with a functional group such as a hydroxyl group, a cyano group, an amino group, a pyrrolidino group, a piperidino group, a morpholino group, and an oxo group, an alkoxy group, or a halogen atom. This shall apply to the alkoxyalkyl group represented by Rb.

As R^{b}, a linear or branched alkyl group, a cycloalkyl group, or an aryl group is preferable, and the linear or branched alkyl group or the cycloalkyl group is more preferable.

Examples of a ring formed by the mutual linking of two R^{b}'s include an alicyclic hydrocarbon group, an aromatic hydrocarbon group, a heterocyclic hydrocarbon, and derivatives thereof.

Examples of the specific structure of the group represented by Formula d-1 include the structures disclosed in paragraph 0466 in US2012/0135348A1 but are not particularly limited thereto.

It is preferable that the compound (DD) has a structure represented by Formula 6.

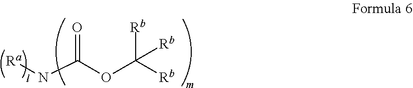

In Formula 6,

1 represents an integer of 0 to 2, and m represents an integer of 1 to 3, satisfying 1+-m=3.

R^{a} represents a hydrogen atom, an alkyl group, a cycloalkyl group, an aryl group, or an aralkyl group. In a case where 1 is 2, two R^{a}'s may be the same as or different from each other and two R^{a}'s may be linked to each other to form a heterocycle, together with the nitrogen atom in the formula. The heterocycle may include a heteroatom other than the nitrogen atom in the formula.

R^{b} has the same meaning as R^{b} in Formula d-1, and preferred examples are also the same.

In Formula 6, the alkyl group, the cycloalkyl group, the aryl group, and the aralkyl group as R^{a} may be each independently substituted with the same groups as the group mentioned above as a group which may be substituted in the alkyl group, the cycloalkyl group, the aryl group, and the aralkyl group as R^{b}.

Specific examples of the alkyl group, the cycloalkyl group, the aryl group, and the aralkyl group (these groups may be substituted with the groups as described above) of R^{a} include the same groups as the specific examples as described above with respect to R^{b}.

Examples of the specific structure of the particularly preferred compound (DD) in the present disclosure include, but are not limited to, the compounds disclosed in paragraph 0475 in US2012/0135348A1.

The oniwn salt compound (DE) (hereinafter also referred to as a “compound (DE)”) having a nitrogen atom in a cation moiety is preferably a compound having a basic moiety including a nitrogen atom in a cation moiety. The basic moiety is preferably an amino group, and more preferably an aliphatic amino group. It is more preferable that all of the atoms adjacent to the nitrogen atom in the basic moiety are hydrogen atoms or carbon atoms. Further, from the viewpoint of improvement of basicity, it is preferable that an electron-withdrawing functional group (a carbonyl group, a sulfonyl group, a cyano group, a halogen atom, and the like) is not directly linked to the nitrogen atom.

Examples of the specific preferred structure of the compound (DE) include, but are not limited to, the compounds disclosed in paragraph 0203 of US2015/0309408A1.

Preferred examples of such other acid diffusion control agents are shown below.

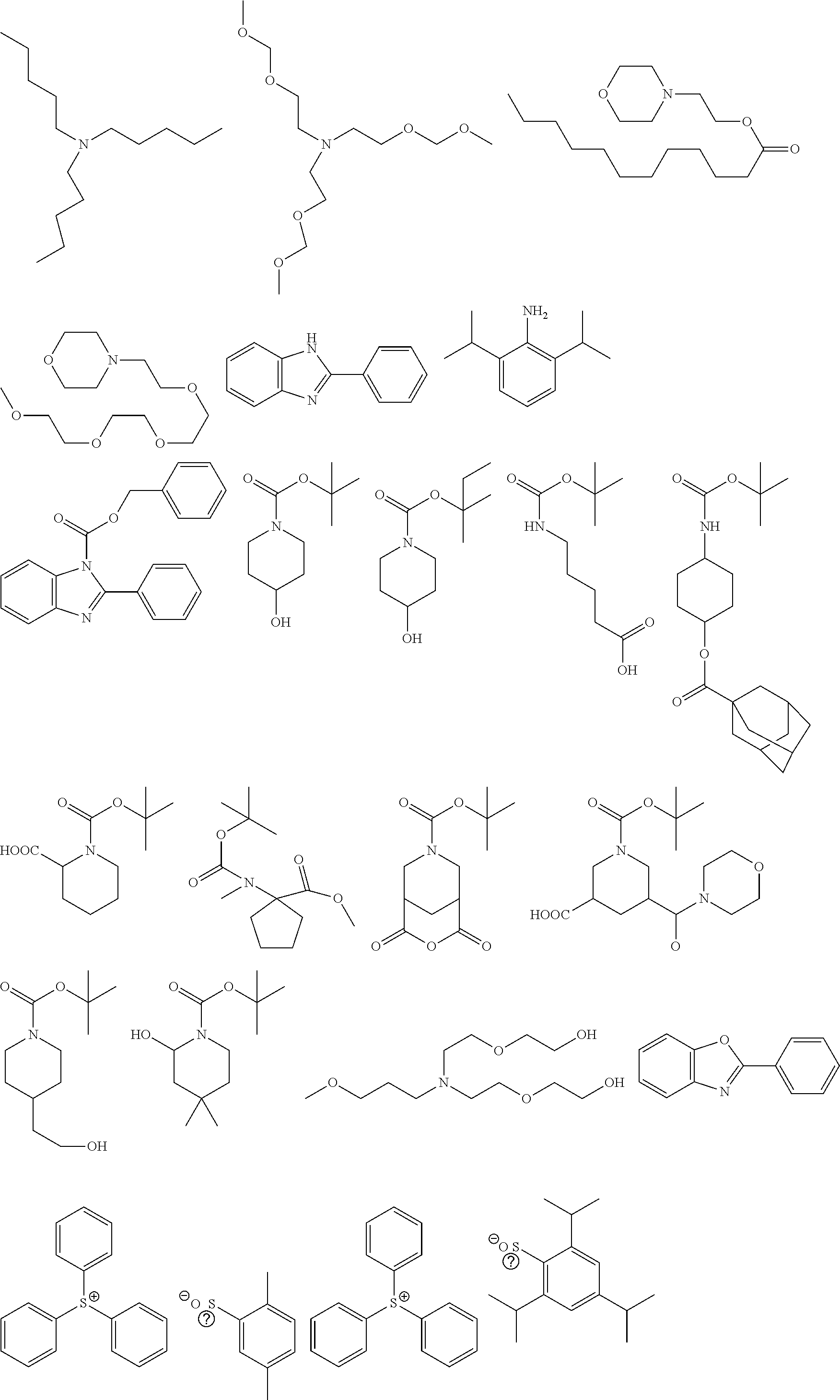

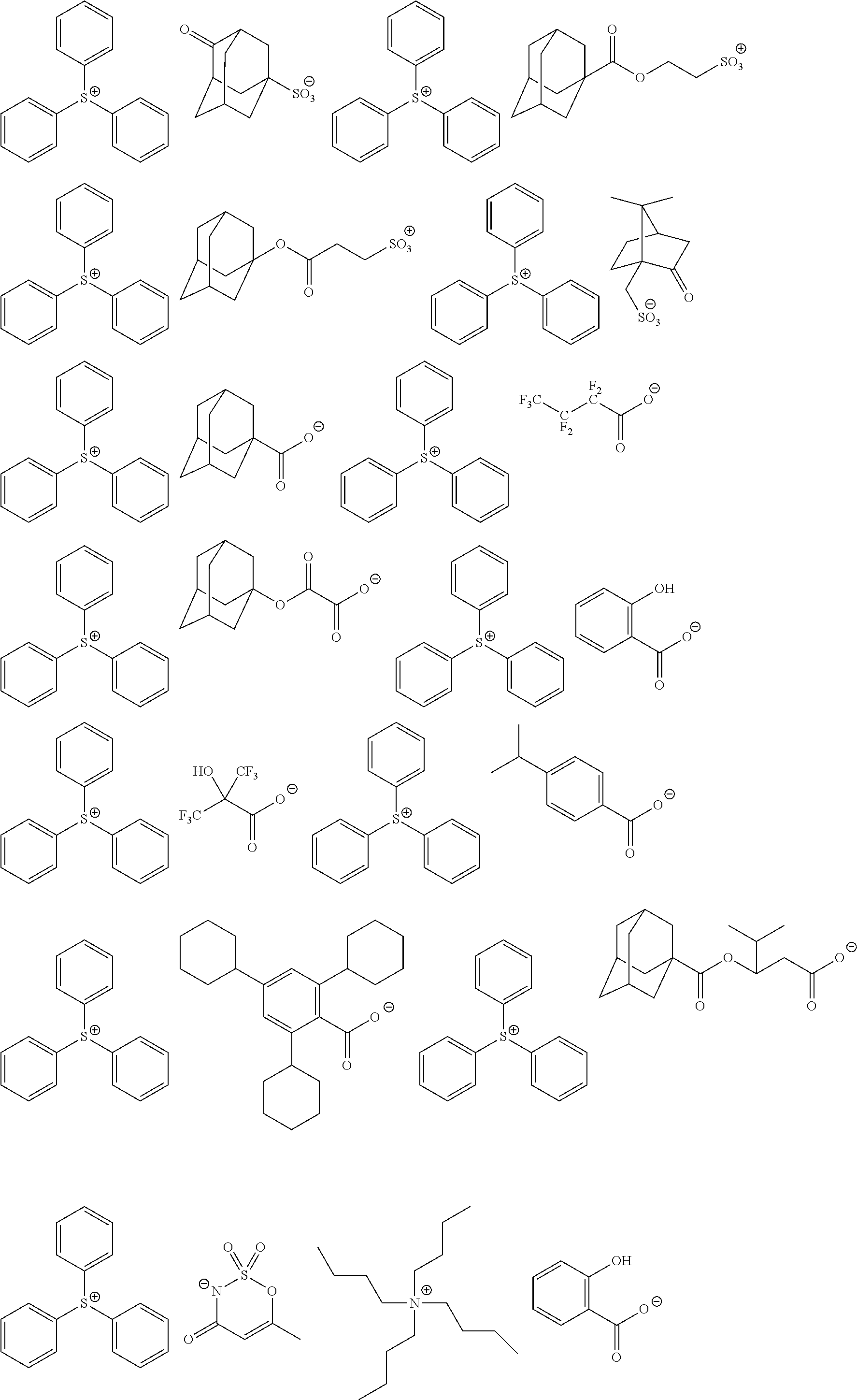

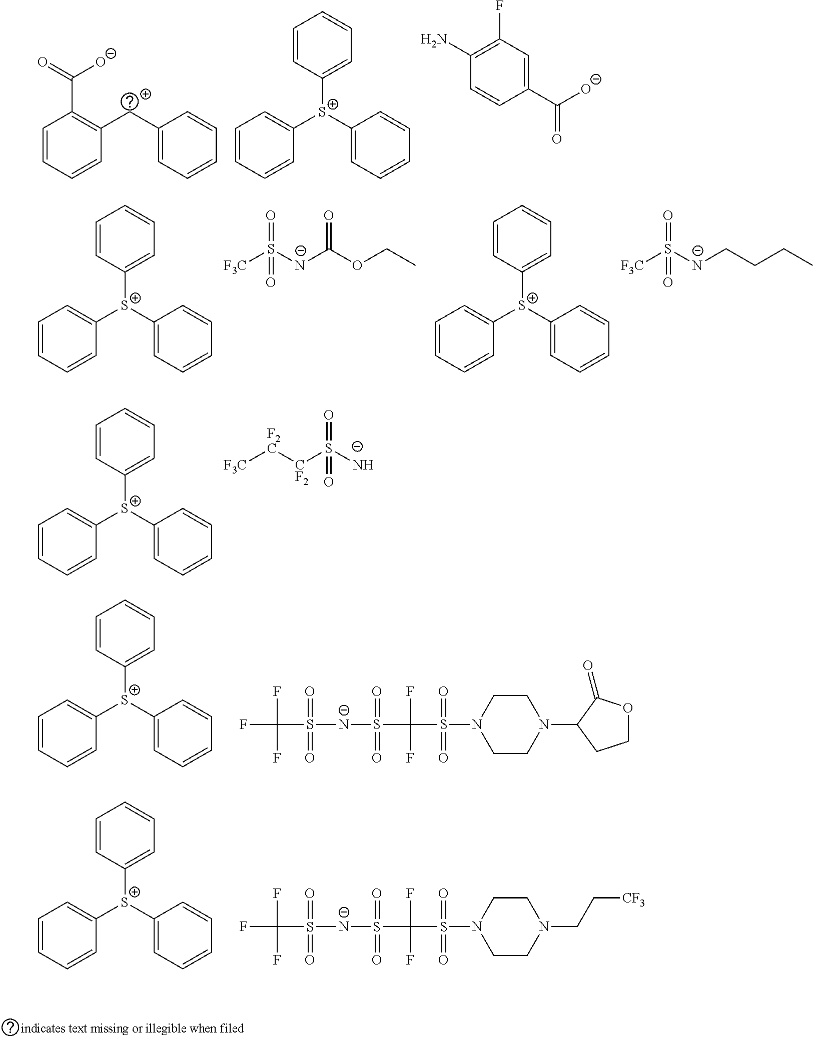

In the photosensitive resin composition according to the present disclosure, such other acid diffusion control agents may be used singly or in combination of two or more kinds thereof.

The content of such another acid diffusion control agent (the total content in a case where a plurality of the acid diffusion control agents are present) in the composition is preferably 0.1% by mass to 10% by mass, and more preferably 0.1% by mass to 5% by mass, with respect to the total solid content of the composition.

<Crosslinking Agent>

The photosensitive resin composition according to the present disclosure may contain a compound that crosslinks a resin by the action of an acid (hereinafter also referred to as a crosslinking agent (G)).

As the crosslinking agent (G), a known compound can be appropriately used. For example, the known compounds disclosed in paragraphs 0379 to 0431 of US2016/0147154A1 and paragraphs 0064 to 0141 of US2016/0282720A1 can be suitably used as the crosslinking agent (G).

The crosslinking agent (G) is a compound having a crosslinkable group which can crosslink a resin, and examples of the crosslinkable group include a hydroxymethyl group, an alkoxymethyl group, an acyloxymethyl group, an alkoxymethyl ether group, an oxirane ring, and an oxetane ring.

The crosslinkable group is preferably the hydroxymethyl group, the alkoxymethyl group, the oxirane ring, or the oxetane ring.

The crosslinking agent (G) is preferably a compound (which also includes a resin) having two or more crosslinkable groups.

The crosslinking agent (G) is more preferably a phenol derivative, a urea-based compound (compound having a urea structure), or a melamine-based compound (compound having a melamine structure), which has a hydroxymethyl group or an alkoxymethyl group.

The crosslinking agents may be used singly or in combination of two or more kinds thereof.

The content of the crosslinking agent (G) is preferably 1% by mass to 50% by mass, more preferably 3% by mass to 40% by mass, and still more preferably 5% by mass to 30% by mass, with respect to the total solid content of the composition.

<Surfactant>

The photosensitive resin composition according to the present disclosure may or may not contain a surfactant. In a case where the composition contains the surfactant, it is preferable that at least one of a fluorine-based surfactant or a silicon-based surfactant (specifically a fluorine-based surfactant, a silicon-based surfactant, or a surfactant having both of a fluorine atom and a silicon atom) is contained.

By incorporating the surfactant into the photosensitive resin composition according to the present disclosure, it is possible to form a pattern which has excellent adhesiveness and decreased development defects with good sensitivity and resolution in a case of using an exposure light source at a wavelength of 250 nm or less, and particularly at a wavelength of 220 nm or less.

Examples of the fluorine-based or silicon-based surfactant include the surfactants described in paragraph 0276 of US2008/0248425A.

In addition, other surfactants other than the fluorine-based or silicon-based surfactant, described in paragraph 0280 of US2008/0248425A, can also be used.

These surfactants may be used singly or in combination of two or more kinds thereof.

In a case where the photosensitive resin composition according to the present disclosure contains a surfactant, the content of the surfactant is preferably 0.0001% by mass to 2% by mass, and more preferably 0.0005% by mass to 1% by mass, with respect to the total solid content of the composition.

On the other hand, by setting the content of the surfactant to 0.0001% by mass or more with respect to the total solid content of the composition, the hydrophobic resin is further unevenly distributed on the surface. Thus, a surface of the actinic ray-sensitive or radiation-sensitive film can be made more hydrophobic, which can enhance water tracking properties upon liquid immersion exposure.

<Other Additives>

The photosensitive resin composition according to the present disclosure may further contain other known additives.

Examples of such other known additive include an acid proliferation agent, a dye, a plasticizer, a light sensitizer, a light absorber, an alkali-soluble resin, a dissolution inhibitor, and a dissolution promoter.

<Preparation Method>

The photosensitive resin composition according to the present disclosure is used by being applied onto a predetermined support (substrate) to be used after dissolving the above-mentioned components in a predetermined organic solvent, and preferably in the mixed solvent, and filtering the solution using a filter. The pore size (pore diameter) of the filter to be used for filtration using the filter is preferably 0.1 μm or less, more preferably 0.05 μm or less, and still more preferably 0.03 μm or less. The filter is preferably a polytetrafluoroethylene-, polyethylene- or nylon-made filter. In the filtration using a filter, circulating filtration may be performed or the filtration may be performed by connecting plural kinds of filters in series or in parallel, as disclosed in JP2002-062667A, for example. In addition, the composition may be filtered in plural times. Furthermore, the composition may be subjected to a deaeration treatment or the like before or after filtration using the filter.

The film thickness of the resist film formed of the photosensitive resin composition according to the present disclosure is not particularly limited, and from the viewpoint of improving the resolving power, the film thickness is preferably 90 nm or less, and more preferably 85 nm or less. Such a film thickness can be set by setting the concentration of the solid contents in the composition to an appropriate range to improve the coating properties or the film forming properties.

<Applications>

The photosensitive resin composition according to the present disclosure is a photosensitive resin composition whose properties change by undergoing a reaction upon irradiation with light. More specifically, the photosensitive resin composition according to the present disclosure relates to an actinic ray-sensitive or radiation-sensitive resin composition which is used in a step of manufacturing a semiconductor such as an integrated circuit (IC), for the manufacture of a circuit board for a liquid crystal, a thermal head, or the like, the manufacture of a mold structure for imprinting, and other photofabri cation steps, or for the production of a planographic printing plate, or an acid-curable composition. A resist pattern formed with the photosensitive resin composition according to the present disclosure can be used in an etching step, an ion implantation step, a bump electrode forming step, a rewiring forming step, microelectromechani cal systems (MEMS), or the like.

(Resist Film)

The resist film according to the present disclosure is a solidified product of the photosensitive resin composition according to the present disclosure.

The solidified product in the present disclosure can be any of products obtained by removing at least a part of the solvent from the photosensitive resin composition according to the present disclosure.

Specifically, the resist film according to the present disclosure is obtained by, for example, applying the photosensitive resin composition according to the present disclosure on a support such as a substrate, followed by drying.

The drying refers to a removal of at least a part of the solvent included in the photosensitive resin composition according to the present disclosure.

The drying method is not particularly limited, known methods are used, and examples thereof include drying under heating (for example, at 70° C. to 130° C. for 30 seconds minute to 300 seconds).

The heating method is not particularly limited, known heating means are used, and examples thereof include a heater, an oven, a hot plate, an infrared lamp, and an infrared laser.

The components included in the resist film according to the present disclosure are the same as the components excluding a solvent among the components included in the photosensitive resin composition according to the present disclosure, and preferred aspects thereof are also the same.

The content of each component included in the resist film according to the present disclosure corresponds to a content of each component, in which a description of “the total solid content” with regard to the content of each component other than the solvent in the photosensitive resin composition according to the present disclosure is replaced by “the total mass of the resist film”.

The thickness of the resist film according to the present disclosure is not particularly limited, and is preferably 50 nm to 150 nm, and more preferably 80 tun to 130 nm.

In addition, in a case where it is intended to form a thick resist film along with a three-dimensional memory device, the thickness is, for example, preferably 2 μm or more, more preferably from 2 μm to 50 μm, and still more preferably from 2 μm to 20 μm,

(Pattern Forming Method)

The pattern forming method according to the present disclosure includes:

a step of exposing the resist film according to the present disclosure with actinic rays (exposing step), and

a step of developing the resist film after the exposing step with a developer (developing step).

Furthermore, the pattern forming method according to the present disclosure may be a method including:

a step of forming a resist film on a support with the photosensitive resin composition according to the present disclosure (film forming step),

a step of exposing the resist film with actinic rays (exposing step), and

a step of developing the resist film after the exposing step with a developer (developing step).

<Film Forming Step>

The pattern forming method according to the present disclosure may include a film forming step. Examples of a method for forming a resist film in the film forming step include a method for forming a resist film by the drying as described in the section of the resist film as described above.

[Support]

The support is not particularly limited, and a substrate which is generally used in a process for manufacturing a semiconductor such as an IC, and a process for manufacturing a circuit board for a liquid crystal, a thermal head, or the like, and other lithographic processes of photofabrication can be used. Specific examples of the support include an inorganic substrate such as silicon, SiO₂, and SiN.

<Exposing Step>

The exposing step is a step of exposing the resist film with light.

The exposing method may be liquid immersion exposure.

The pattern forming method according to the present disclosure may include the exposing steps plural times.

A type of the light (actinic rays or radiation) used for exposure may be selected in consideration of characteristics of a photoacid generator, a pattern shape to be obtained, and the like, but examples of the light include infrared rays, visible light, ultraviolet rays, far ultraviolet rays, extreme ultraviolet rays (EUV), X-rays, and electron beams, and the far ultraviolet rays are preferable.

For example, actinic rays at a wavelength of 250 nm or less are preferable, actinic rays at a wavelength of 220 nm or less are more preferable, and actinic rays at a wavelength of 1 to 200 nm are still more preferable.

Specific examples of light used include a KrF excimer laser (248 nm), an ArF excimer laser (193 nm), an F₂ excimer laser (157 nm), X-rays, EUV (13 nm), and electron beams, and the ArF excimer laser, EUV, or the electron beams are preferable.

It is preferable that the exposure in the step of performing exposure is performed by liquid immersion exposure with an argon fluoride laser or the exposure in the step of performing exposure is performed by exposure using a krypton fluoride laser.

The exposure dose is preferably 5 mJ/ctn² to 200 mJ/cm², and more preferably 10 mJ/cm² to 100 mJ/cm².

<Developing Step>

The developer used in the developing step may be an alkali developer or a developer containing an organic solvent (hereinafter also referred to as an organic developer), or is preferably an aqueous alkali solution.

[Alkali Developer]

As the alkali developer, a quaternary ammonium salt typified by tetramethylammonium hydroxide is preferably used, but in addition to the developer, an aqueous alkali solution such as an inorganic alkali, primary to tertiary amines, alkanolamine, and cyclic amine can also be used.

In addition, the alkali developer may contain an appropriate amount of at least one of alcohols or a surfactant. The alkali concentration of the alkali developer is preferably 0.1% by mass to 20% by mass. The pH of the alkali developer is preferably 10 to 15.

A period for performing development using the alkali developer is preferably 10 seconds to 300 seconds.

The alkali concentration, the pH, and the developing time using the alkali developer can be appropriately adjusted depending on a pattern formed.

[Organic Developer]

As the organic developer, a developer containing at least one organic solvent selected from the group consisting of a ketone-based solvent, an ester-based solvent, an alcohol-based solvent, an amide-based solvent, an ether-based solvent, and a hydrocarbon-based solvent is preferable.

Ketone-Based Solvent

Examples of the ketone-based solvent include 1-octanone, 2-octanone, 1-nonanone, 2-nonanone, acetone, 2-heptanone (methyl amyl ketone), 4-heptanone, 1-hexanone, 2-hexanone, diisobutyl ketone, cyclohexanone, methylcyclohexanone, phenyl acetone, methyl ethyl ketone, methyl isobutyl ketone, acetyl acetone, acetonyl acetone, ionone, diacetonyl alcohol, acetyl carbinol, acetophenone, methyl naphthyl ketone, isophorone, and propylene carbonate.

Ester-Based Solvent

Examples of the ester-based solvent include methyl acetate, butyl acetate, ethyl acetate, isopropyl acetate, pentyl acetate, isopentyl acetate, amyl acetate, propylene glycol monomethyl ether acetate, ethylene glycol monoethyl ether acetate, diethylene glycol monobutyl ether acetate, diethylene glycol monoethyl ether acetate ethyl-3-ethoxypropionate, 3-methoxybutyl acetate, 3-methyl-3-methoxybutyl acetate, methyl formate, ethyl formate, butyl formate, propyl formate, ethyl lactate, butyl lactate, propyl lactate, butyl butyrate, methyl 2-hydroxyisobutyrate, isoamyl acetate, isobutyl isobutyrate, and butyl propionate.

Other Solvents

As the alcohol-based solvent, the amide-based solvent, the ether-based solvent, and the hydrocarbon-based solvent, the solvents disclosed in paragraphs 0715 to 0718 of US2016/0070167A1 can be used.

A plurality of the above-mentioned solvents may be mixed or the solvent may be used in admixture with a solvent other than those described above or with water. The moisture content in the entire developer is preferably less than 50% by mass, more preferably less than 20% by mass, and still more preferably less than 10% by mass, and particularly preferably, moisture is not substantially contained.

The content of the organic solvent in the organic developer is preferably from 50% by mass to 100% by mass, more preferably from 80% by mass to 100% by mass, still more preferably from 90% by mass to 100% by mass, and particularly preferably from 95% by mass to 100% by mass, with respect to the total amount of the developer.

Surfactant

The organic developer can contain an appropriate amount of a known surfactant, as necessary.

The content of the surfactant is preferably 0.001% by mass to 5% by mass, more preferably 0.005% by mass to 2% by mass, and still more preferably 0.01% by mass to 0.5% by mass, with respect to the total amount of the developer.

Acid Diffusion Control Agent

The organic developer may include the above-mentioned acid diffusion control agent.

[Developing Method]

As the developing method, for example, a method in which a substrate is immersed in a tank filled with a developer for a certain period of time (a dip method), a method in which a developer is heaped up onto the surface of a substrate by surface tension, and then left to stand for a certain period of time (a puddle method), a method in which a developer is sprayed on the surface of a substrate (a spray method), and a method in which a developer is continuously jetted onto a substrate spun at a constant rate while scanning a developer jetting nozzle at a constant rate (a dynamic dispense method) can be applied.

A step of performing development using an aqueous alkali solution (an alkali developing step) and a step of performing development using a developer including an organic solvent (an organic solvent developing step) may be combined. Thus, a finer pattern can be formed since a pattern can be formed by keeping only a region with an intermediate exposure intensity from not being dissolved.

<Prebaking Step and Post-Exposure Baking Step>

It is preferable that the pattern forming method according to the present disclosure includes a prebaking (PB) step before the exposing step.

The pattern forming method according to the present disclosure may include the prebaking steps a plurality of times.

It is preferable that the pattern forming method according to the present disclosure includes a post-exposure baking (PEB) step after the exposing step and before the developing step.

The pattern forming method according to present disclosure may include the post-exposure baking steps a plurality of times.

The heating temperature is preferably 70° C. to 130° C., and more preferably 80° C. to 120° C. in any of the prebaking step and the post-exposure baking step.

The heating time is preferably 30 seconds to 300 seconds, more preferably 30 seconds to 180 seconds, and still more preferably 30 seconds to 90 seconds in any of the prebaking step and the post-exposure baking step.

Heating can be performed using a means comprised in an exposure device and a development device, or may also be performed using a hot plate or the like.

<Step of Forming Resist Underlayer Film>

The pattern forming method according to the present disclosure may further include a step of forming a resist underlayer film (resist underlayer film forming step) before the film forming step.

The resist underlayer film forming step is a step of forming a resist underlayer film (for example, spin on glass (SOG), spin on carbon (SOC), and an antireflection film) between the resist film and the support. For the resist underlayer film, known organic or inorganic materials can be appropriately used.

<Protective Film Forming Step>

The pattern forming method according to the present disclosure may further include a step of forming a protective film (protective film forming step) before the developing step.

The protective film forming step is a step of forming a protective film (topcoat) on the upper layer of the resist film.

As the protective film, a known material can be appropriately used. The compositions for forming a protective film disclosed in, for example, US2007/0178407A, US2008/0085466A, US2007/0275326A, US2016/0299432A, US2013/0244438A, or WO2016/157988A can be suitably used. A composition for forming a protective film preferably includes the above-mentioned acid diffusion control agent.

The protective film may also be formed on the upper layer of the resist film containing the above-mentioned hydrophobic resin.

<Rinsing Step>

The pattern forming method according to the present disclosure preferably includes a step of performing washing with a rinsing liquid (rinsing step) after the developing step.

[Case of Developing Step Using Alkali Developer]

As the rinsing liquid used in the rinsing step after the developing step using an alkali developer, for example, pure water can be used. Pure water may contain an appropriate amount of a surfactant. In this case, after the developing step or the rinsing step, a treatment for removing the developer or the rinsing liquid adhering on a pattern by a supercritical fluid may be added. In addition, after the rinsing treatment or the treatment using a supercritical fluid, a heating treatment for removing moisture remaining in the pattern may be performed.

[Case of Developing Step Using Organic Developer]

The rinsing liquid used in the rinsing step after the developing step using a developer including an organic solvent is not particularly limited as long as the rinsing liquid does not dissolve the resist pattern, and a solution including a common organic solvent can be used. As the rinsing liquid, a rinsing liquid containing at least one organic solvent selected from the group consisting of a hydrocarbon-based solvent, a ketone-based solvent, an ester-based solvent, an alcohol-based solvent, an amide-based solvent, and an ether-based solvent is preferably used.

Specific examples of the hydrocarbon-based solvent, the ketone-based solvent, the ester-based solvent, the alcohol-based solvent, the amide-based solvent, and the ether-based solvent are the same solvents as those described for the developer including an organic solvent.

As the rinsing liquid used in the rinsing step in this case, a rinsing liquid containing a monohydric alcohol is more preferable.

Examples of the monohydric alcohol used in the rinsing step include linear, branched, or cyclic monohydric alcohols. Specific examples thereof include 1-butanol, 2-butanol, 3-methyl-1-butanol, tert-butyl alcohol, 1-pentanol, 2-pentanol, 1-hexanol, 4-methyl-2-pentanol, 1-heptanol, 1-octanol, 2-hexanol, cyclopentanol, 2-heptanol, 2-octanol, 3-hexanol, 3-heptanol, 3-octanol, 4-octanol, and methyl isobutyl carbinol. Examples of the monohydric alcohol having 5 or more carbon atoms include 1-hexanol, 2-hexanol, 4-methyl-2-pentanol, 1-pentanol, 3-methyl-1-butanol, and methyl isobutyl carbinol.

The respective components in plural number may be mixed or the components may be used in admixture with an organic solvent other than the above solvents.

The moisture content in the rinsing liquid is preferably 10% by mass or less, more preferably 5% by mass or less, and still more preferably 3% by mass or less. By setting the moisture content to 10% by mass or less, good development characteristics can be obtained.

The rinsing liquid may contain an appropriate amount of a surfactant.

In the rinsing step, the substrate that has been subjected to development using an organic developer is subjected to a washing treatment using a rinsing liquid including an organic solvent. A method for the washing treatment method is not particularly limited, but for example, a method in which a rinsing liquid is continuously jetted on a substrate rotated at a constant rate (a rotation application method), a method in which a substrate is immersed in a tank filled with a rinsing liquid for a certain period of time (a dip method), and a method in which a rinsing liquid is sprayed on a substrate surface (a spray method) can be applied. Among those, it is preferable that a washing treatment is performed using the rotation application method, and a substrate is rotated at a rotation speed of 2,000 rpm to 4,000 revolutions per minute (rpm) after washing, thereby removing the rinsing liquid from the substrate. Furthermore, it is also preferable that the method includes a baking step after the rinsing step (post-baking). The developer and the rinsing liquid remaining between and inside the patterns are removed by the baking step. In the baking step after the rinsing step, the heating temperature is preferably 40° C. to 160° C., and more preferably 70° C. to 95° C. The heating time is preferably 10 seconds to 3 minutes, and more preferably 30 seconds to 90 seconds.

<Impurities of Various Materials>

It is preferable that various materials (for example, a resist solvent, a developer, a rinsing liquid, a composition for forming an antireflection film, and a composition for forming a topcoat) used in the photosensitive resin composition according to the present disclosure resin composition and the pattern forming method according to the present disclosure do not include impurities such as metal components, isomers, and residual monomers. The content of the impurities included in these materials is preferably 1 ppm or less, more preferably 100 ppt or less, and still more preferably 10 ppt by mass or less, and particularly preferably, the impurities are not substantially included (no higher than a detection limit of a measurement device)

Examples of a method for removing impurities such as metals from the various materials include filtration using a filter. As for the filter pore diameter, the pore size is preferably 10 nm or less, more preferably 5 nm or less, and still more preferably 3 nm or less. As for the materials of a filter, a polytetrafluoroethylene-made filter, a polyethylene-made filter, and a nylon-made filter are preferable. As the filter, a filter which had been washed with an organic solvent in advance may be used. In the step of filtration using a filter, plural kinds of filters connected in series or in parallel may be used. In a case of using the plural kinds of filters, a combination of filters different in at least one of pore diameters or materials may be used. In addition, various materials may be filtered plural times, and the step of filtering plural times may be a circulatory filtration step. As the filter, a filter having a reduced amount of elutes as disclosed in JP2016-201426A is preferable.

In addition to the filtration using a filter, removal of impurities by an adsorbing material may be performed, or a combination of filtration using a filter and an adsorbing material may be used. As the adsorbing material, known adsorbing materials can be used, and for example, inorganic adsorbing materials such as silica gel and zeolite, and organic adsorbing materials such as activated carbon can be used. Examples of the metal adsorbing agent include those disclosed in JP2016-206500A.

In addition, as a method for removing the impurities such as metals included in various materials, metal content selects the less material as a raw material constituting the various materials, performing filtering using a filter of the raw material constituting the various materials, equipment the inner and a method such as performing distillation under conditions suppressing as much as possible equal to contamination is lined with TEFLON (registered trademark). Preferred conditions in the filtering using a filter to be performed on the raw material constituting the various materials are similar to the above-mentioned conditions.

In order to prevent impurities from being incorporated, it is preferable that the above-mentioned various materials are stored in the container described in US2015/0227049A, JP2015-123351A, JP2017-013804A, or the like.

<Improvement of Surface Roughness>

A method for improving the surface roughness of a pattern may be applied to a pattern formed by the pattern forming method according to the present disclosure. Examples of the method for improving the surface roughness of a pattern include the method of treating a resist pattern by plasma of a hydrogen-containing gas disclosed in US2015/0104957A. In addition, known methods as described in JP2004-2354684, US2010/00202974, and Proc. of SPIE Vol. 8328 83280N-1 “EUV Resist Curing Technique for LWR Reduction and Etch Selectivity Enhancement” may be applied.

In addition, a pattern formed by the method can be used as a core material (core) of the spacer process disclosed in JP1991-270227A (JP-H03-270227A) and US2013/0209941A, for example.

(Method for Manufacturing Electronic Device)

The method for manufacturing an electronic device according to the present disclosure includes the pattern forming method according to the present disclosure. An electronic device manufactured by the method for manufacturing an electronic device according to the present disclosure is suitably mounted on electric or electronic equipment (for example, home electronics, office automation (OA)-related equipment, media-related equipment, optical equipment, and telecommunication equipment).

### EXAMPLES

Hereinbelow, embodiments of the present invention will be described in more detail with reference to Examples. The materials, the amounts of materials used, the proportions, the treatment details, the treatment procedure, or the like shown in the following Examples may be appropriately modified as long as the modifications do not depart from the spirit of the embodiments of the present invention. Therefore, the scope of the embodiments of the present invention is not particularly limited to the specific examples shown below. In addition, “parts” and “%” are on a mass basis unless otherwise specified.

<Synthesis of Resin>

[Synthesis of Resin A-I]

Under a nitrogen gas stream, 40 parts by mass of cyclohexanone with respect to 100 parts by mass of the total mass of the monomers was put into a three-neck flask and heated at 80° C. A solution obtained by dissolving 50 molar equivalents of t-butyl methacrylate, 50 molar equivalents of 2-methacryloxy-γ-butyrolactone, and 8% by mole of a polymerization initiator V-601 (manufactured by Wako Pure Chemical Industries, Ltd.) with respect to the monomers in 340 parts by mass of cyclohexanone was added dropwise thereto for 6 hours. After completion of the dropwise addition, the mixture was further reacted at 80° C. for 2 hours. The reaction solution was left to be cooled and then added dropwise to a mixed liquid of 3,500 parts by mass of hexane/875 parts by mass of ethyl acetate for 20 minutes, and the precipitated powder was collected by filtration and dried to obtain a resin A-1. The obtained resin had a weight-average molecular weight of 12,000 in terms of polystyrene as a standard and a dispersity (Mw/Mn) of 1.5.

[Synthesis of Resins A-2 to A-15]

Resins A-2 to A-15 were each synthesized by the same method as for the synthesis of the resin A-1, except that the monomer used was changed to the monomer and the content, each as described in Table 4 in the synthesis of the resin A-1.

TABLE 4

| | Constitutional unit 1 | | Constitutional unit 2 | | Constitutional unit 3 | | Constitutional unit 4 | | Constitutional unit 5 | | | |
|---|---|---|---|---|---|---|---|---|---|---|---|---|
| | | Content | | Content | | Content | | Content | | Content | | |
| | | ratio | | ratio | | ratio | | ratio | | ratio | | |
| | Monomer | (% by | Monomer | (% by | Monomer | (% by | Monomer | (% by | Monomer | (% by | | Mw/ |
| | used | mole) | used | mole) | used | mole) | used | mole) | used | mole) | Mw | Mn |
| Resin A-1 | MB-1 | 50 | MA-1 | 50 | | | | | | | 12,000 | 1.5 |
| Resin A-2 | MB-2 | 40 | MA-2 | 30 | MA-3 | 30 | | | | | 8,000 | 1.7 |
| Resin A-3 | MB-3 | 30 | MB-4 | 20 | MA-4 | 40 | MC-1 | 10 | | | 15,000 | 1.3 |
| Resin A-4 | MB-3 | 10 | MB-5 | 20 | MA-5 | 40 | MA-8 | 30 | | | 12,000 | 1.4 |
| Resin A-5 | MB-4 | 30 | MB-8 | 10 | MA-9 | 20 | MA-10 | 40 | | | 7,500 | 1.6 |
| Resin A-6 | MB-4 | 50 | MA-3 | 25 | MA-4 | 25 | | | | | 13,000 | 1.7 |
| Resin A-7 | MB-3 | 10 | MB-4 | 40 | MA-3 | 40 | MA-5 | 10 | | | 10,000 | 1.5 |
| Resin A-8 | MB-3 | 10 | MB-6 | 40 | MA-3 | 20 | MA-5 | 30 | | | 9,000 | 1.4 |
| Resin A-9 | MB-2 | 20 | MB-4 | 30 | MA-3 | 40 | MA-5 | 10 | | | 8,500 | 1.6 |
| Resin A-10 | MB-4 | 20 | MB-6 | 30 | MA-3 | 30 | MA-5 | 20 | | | 9,000 | 1.5 |
| Resin A-11 | MB-3 | 40 | MA-5 | 30 | MA-12 | 30 | | | | | 8,500 | 1.7 |
| Resin A-12 | MB-2 | 20 | MB-3 | 20 | MA-3 | 30 | MA-5 | 15 | MA-12 | 15 | 8,500 | 1.4 |
| Resin A-13 | MB-4 | 15 | MB-6 | 20 | MA-6 | 30 | MA-11 | 25 | MC-2 | 10 | 10,000 | 1.5 |
| Resin A-14 | MB-3 | 20 | MB-7 | 20 | MA-7 | 45 | MC-3 | 15 | | | 14,000 | 1.3 |
| Resin A-15 | MB-1 | 20 | MB-7 | 20 | MA-9 | 50 | MC-4 | 10 | | | 9,500 | 1.7 |

Details of the respective monomers described in Table 4 are shown below

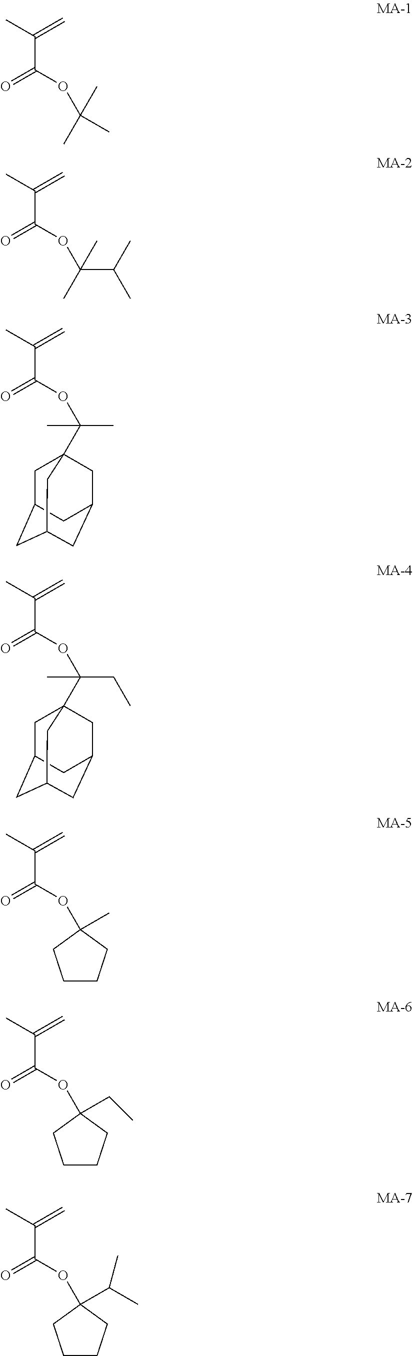

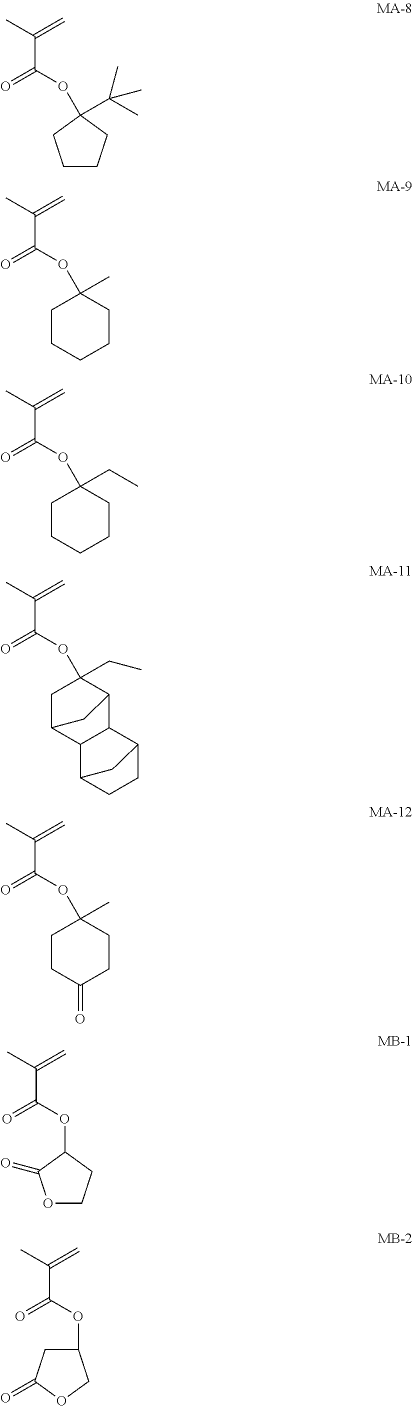

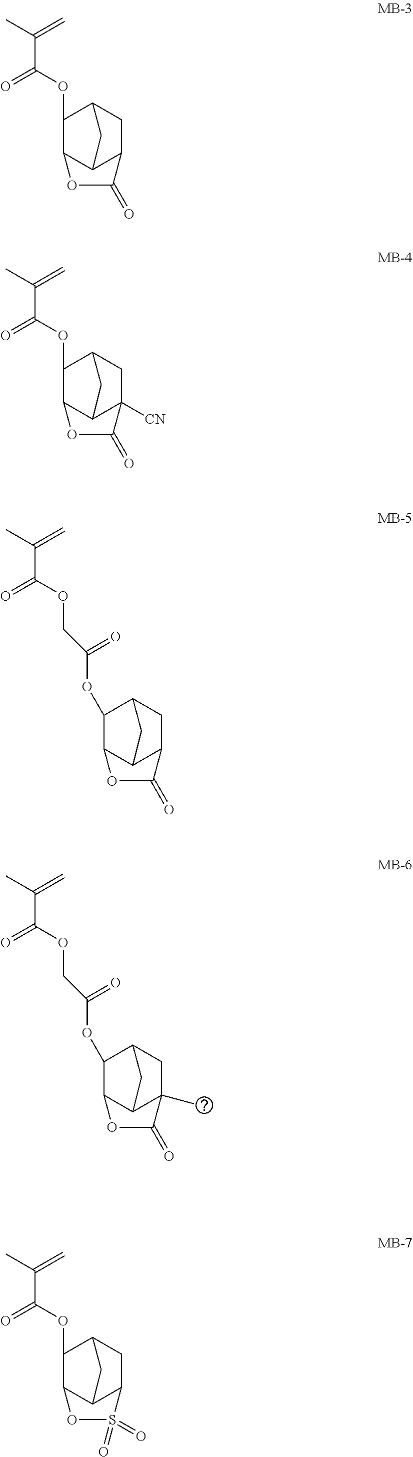 [SMILES: C=C(C)C(=O)OC1C2CC3C(=O)OC1C3C2 ; C=C(C)C(=O)OC1C2CC3C1OC(=O)C3(C#N)C2 ; C=C(C)C(=O)OCC(=O)OC1C2CC3C(=O)OC1C3C2 ; C=C(C)C(=O)OCC(=O)OC1C2CC3C1OC(=O)C3(C)C2 ; C=C(C)C(=O)OC1C2CC3C1OS(=O)(=O)C3C2]

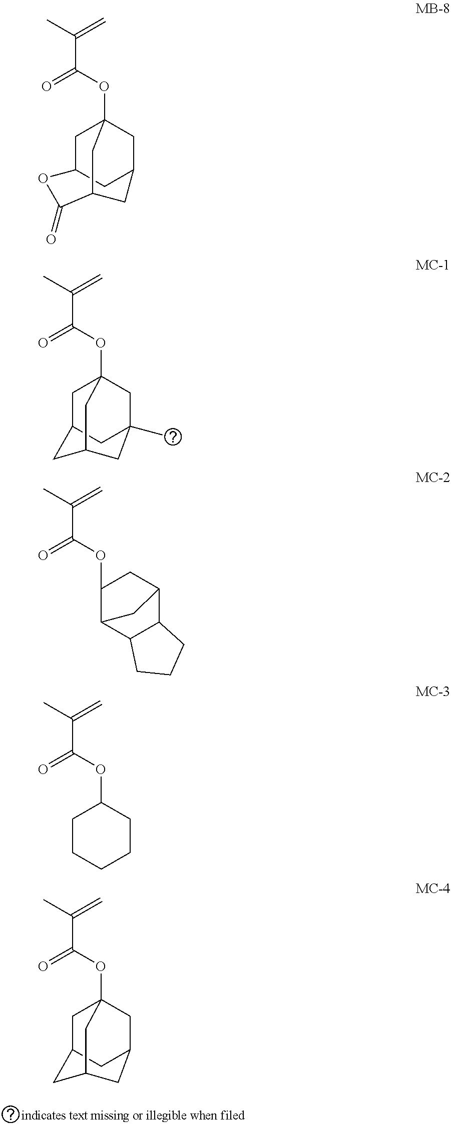

<Synthesis of Fluorine-Containing Resins and Resins for Topcoat]

[Synthesis of Fluorine-Containing Resins E-1 to E-11 and Resins PT-1 to PT-3 for Topcoat]

Fluorine-containing resin E-1 to E-11 and resins PT-1 to PT-3 for a topcoat were each synthesized by the same method as for the synthesis of the resin A-1, except that the monomers used were changed to the monomers and the contents described in Table 5 below in the synthesis of the resin A-1.

TABLE 5

| | Constitutional unit 1 | | Constitutional unit 2 | | Constitutional unit 3 | | Constitutional unit 4 | | | |
|---|---|---|---|---|---|---|---|---|---|---|
| | Monomer | Content ratio | Monomer | Content ratio | Monomer | Content ratio | Monomer | Content ratio | | |
| | used | (% by mole) | used | (% by mole) | used | (% by mole) | used | (% by mole) | Mw | Mw/Mn |
| Resin E-1 | ME-3 | 60 | ME-4 | 40 | | | | | 10,000 | 1.4 |
| Resin E-2 | ME-15 | 50 | ME-1 | 50 | | | | | 12,000 | 1.5 |
| Resin E-3 | ME-2 | 40 | ME-13 | 50 | ME-9 | 5 | ME-20 | 5 | 6,000 | 1.3 |
| Resin E-4 | ME-19 | 50 | ME-14 | 50 | | | | | 9,000 | 1.5 |
| Resin E-5 | ME-10 | 50 | ME-2 | 50 | | | | | 15,000 | 1.5 |
| Resin E-6 | ME-17 | 50 | ME-15 | 50 | | | | | 10,000 | 1.5 |
| Resin E-7 | ME-7 | 100 | | | | | | | 23,000 | 1.7 |
| Resin E-8 | ME-5 | 100 | | | | | | | 13,000 | 1.5 |
| Resin E-9 | ME-6 | 50 | ME-16 | 50 | | | | | 10,000 | 1.7 |
| Resin E-10 | ME-13 | 10 | ME-18 | 85 | ME-9 | 5 | | | 11,000 | 1.4 |
| Resin E-11 | ME-8 | 80 | ME-11 | 20 | | | | | 13,000 | 1.4 |
| Resin PT-1 | ME-2 | 40 | ME-11 | 30 | ME-9 | 30 | | | 8,000 | 1.6 |
| Resin PT-2 | ME-2 | 50 | ME-12 | 40 | ME-3 | 10 | | | 5,000 | 1.5 |
| Resin PT-3 | ME-3 | 30 | ME-4 | 70 | | | | | 8,500 | 1.7 |

Details of the respective monomers described in Table 5 are shown below.

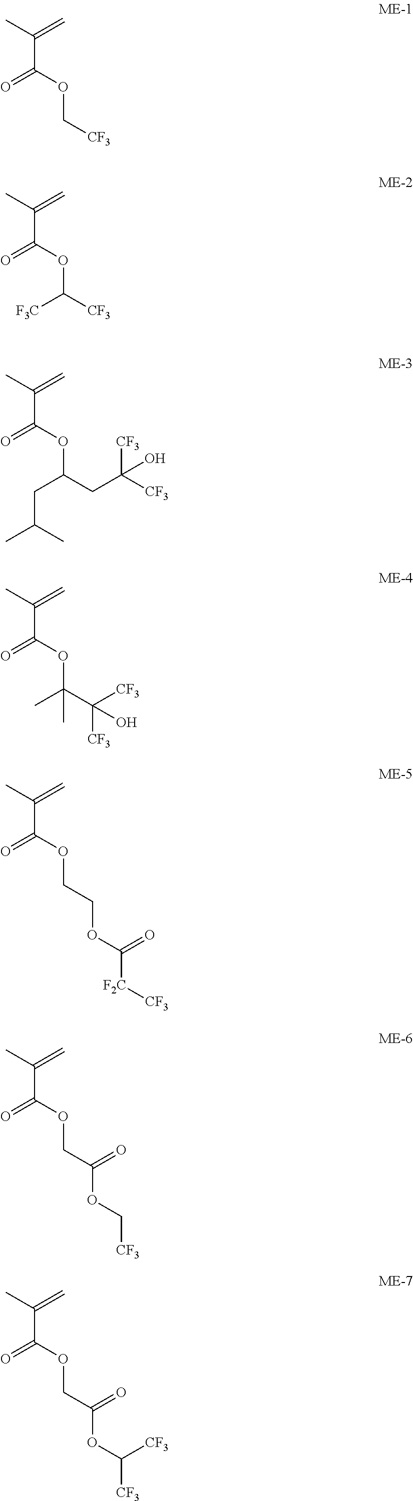

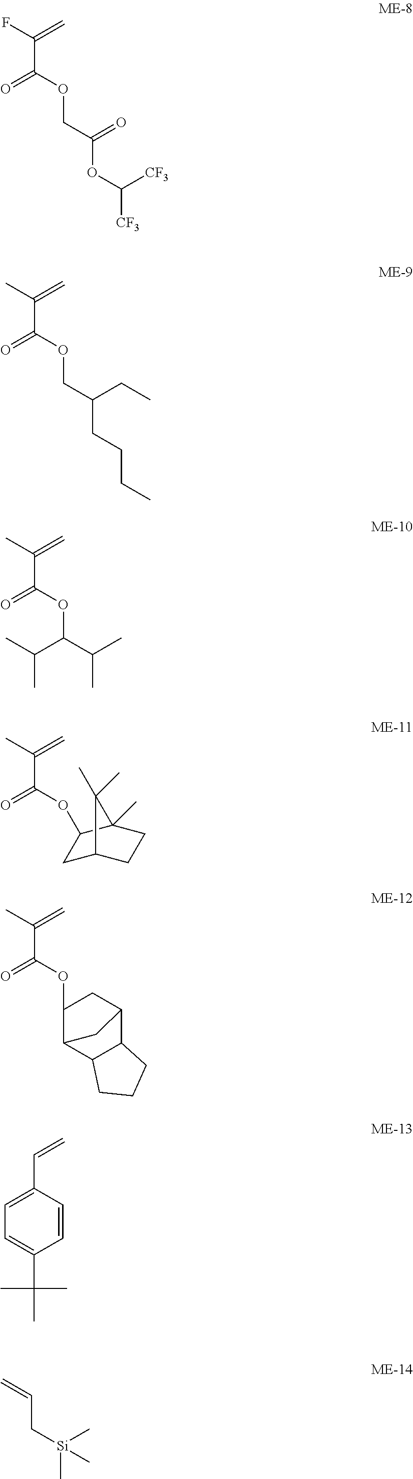

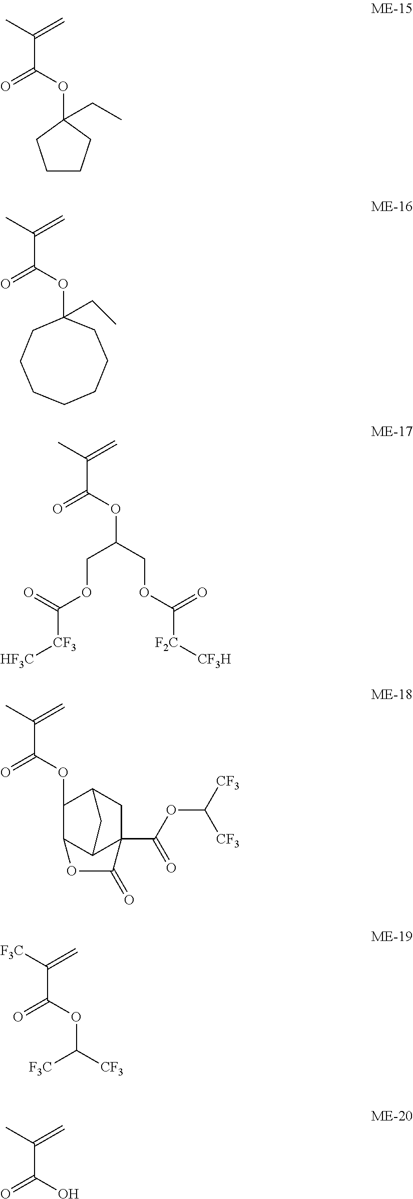

### Examples 1 to 25 and Comparative Examples 1 to 5

<Preparation of Photosensitive Resin Composition>

The components shown in Table 6 were dissolved in a solvent to prepare a solution having a concentration of the solid contents of 4.5% by mass for each component, and this solution was filtered through a polyethylene filter having a pore size of 0.03 μm to prepare a photosensitive resin composition. The prepared photosensitive resin composition was evaluated by the following method and the evaluation results are shown in Table 9.

TABLE 6

| | Resin having | | | | | | | | | | | |
|---|---|---|---|---|---|---|---|---|---|---|---|---|
| | constitutional | | | | | | Compound | | | | | |
| | unit having acid- | | | | | | represented by | | | | | |
| Photosensitive | decomposable group | | Photoacid generator | | | | Formula D | | Fluorine-containing resin | | | |
| resin | | Parts by | | Parts by | | Parts by | | Parts by | | Parts by | | Parts by |
| composition | Type | mass | Type | mass | Type | mass | Type | mass | Type | mass | Type | mass |
| Re-1 | A-1 | 78.9 | PAG-1 | 10 | | | D-1 | 1.1 | E-1 | 2.5 | | |
| Re-2 | A-2 | 77.2 | PAG-2 | 13 | | | D-2 | 1.3 | E-2 | 1.5 | | |
| Re-3 | A-3 | 77.9 | PAG-3 | 11 | | | D-3 | 0.8 | E-3 | 3.0 | | |
| Re-4 | A-4 | 79.5 | PAG-4 | 12 | | | D-4 | 2.0 | E-4 | 4.0 | | |
| Re-5 | A-5 | 80.8 | PAG-5 | 12 | | | D-5 | 1.2 | E-5 | 2.0 | | |
| Re-6 | A-6 | 81.4 | PAG-3 | 9 | PAG-6 | 5 | D-6 | 1.1 | E-6 | 1.5 | | |
| Re-7 | A-7 | 80.0 | PAG-2 | 13 | PAG-7 | 4 | D-7 | 1.5 | E-7 | 1.5 | | |
| Re-8 | A-8 | 79.7 | PAG-2 | 11 | PAG-8 | 6 | D-8 | 1.3 | E-8 | 2.0 | | |
| Re-9 | A-9 | 78.0 | PAG-2 | 10 | PAG-9 | 6 | D-9 | 1.0 | E-9 | 2.5 | | |
| Re-10 | A-10 | 77.4 | PAG-10 | 9 | | | D-10 | 1.1 | E-10 | 1.0 | E-11 | 4.0 |
| Re-11 | A-11 | 85.2 | PAG-11 | 8 | | | D-11 | 1.1 | E-11 | 2.5 | | |
| Re-12 | A-12 | 76.5 | PAG-1 | 9 | PAG-12 | 6 | D-12 | 1.0 | E-10 | 1.0 | E-3 | 2.5 |
| Re-13 | A-13 | 81.6 | PAG-4 | 13 | | | D-13 | 1.9 | — | | | |
| Re-14 | A-14 | 86.8 | PAG-10 | 8 | | | D-2 | 1.2 | — | | | |
| Re-15 | A-15 | 85.6 | PAG-11 | 10 | | | D-11 | 0.9 | — | | | |
| Re-16 | A-7 | 80.7 | PAG-2 | 10 | | | D-1 | 0.8 | E-10 | 0.5 | E-11 | 2.0 |
| Re-17 | A-7 | 81.7 | PAG-3 | 10 | | | D-10 | 0.8 | E-2 | 0.5 | E-3 | 1.0 |
| Re-18 | A-8 | 78.0 | PAG-1 | 12 | | | D-2 | 1.5 | E-6 | 1.0 | E-9 | 2.5 |
| Re-19 | A-9 | 86.0 | PAG-4 | 10 | | | D-l | 1.0 | E-6 | 1.0 | | |
| Re-20 | A-10 | 80.2 | PAG-3 | 9 | | | D-10 | 1.0 | E-9 | 0.5 | E-10 | 2.5 |
| Re-21 | A-11 | 83.8 | PAG-10 | 11 | | | D-4 | 1.2 | E-12 | 1.0 | | |
| Re-22 | A-12 | 83.8 | PAG-12 | 11 | | | D-8 | 1.2 | E-4 | 1.0 | | |
| Re-23 | A-7 | 84.7 | PAG-5 | 10 | | | D-10 | 0.8 | E-1 | 1.5 | E-8 | 1.0 |
| Re-24 | A-7 | 85.7 | PAG-6 | 10 | | | D-13 | 0.8 | E-7 | 1.5 | | |
| Re-25 | A-7 | 85.7 | PAG-8 | 10 | | | D-3 | 0.8 | E-8 | 1.5 | | |
| Re-A | A-7 | 79.2 | PAG-1 | 10 | | | D-14 | 0.8 | E-9 | 4.0 | | |
| Re-B | A-10 | 80.2 | PAG-3 | 9 | | | D-15 | 1.0 | E-10 | 3.0 | | |
| Re-C | A-11 | 85.3 | PAG-4 | 11 | | | D-16 | 1.2 | E-5 | 2.5 | | |
| Re-D | A-7 | 84.7 | PAG-9 | 10 | | | D-17 | 0.8 | E-7 | 1.5 | E-8 | 1.0 |
| Re-E | A-7 | 80.2 | PAG-11 | 10 | | | D-18 | 0.8 | E-9 | 1.5 | E-10 | 1.5 |

| | Additives such as | | Other acid | | | | | |
|---|---|---|---|---|---|---|---|---|
| | low-molecular-weight | | diffusion | | | | | |
| Photosensitive | ester compound | | control agents | | Solvent | | | |
| resin | | Parts by | | Parts by | | Mixing ratio | | Mixing ratio |
| composition | Type | mass | Type | mass | Type | (mass) | Type | (mass) |
| Re-1 | F-1 | 4 | G-1 | 3.5 | C-1 | 80 | C-2 | 20 |
| Re-2 | F-2 | 3 | G-2 | 4 | C-1 | 75 | C-3 | 25 |
| Re-3 | F-3 | 4 | G-3 | 3.3 | C-1 | 88 | C-4 | 12 |
| Re-4 | F-4 | 2.5 | | | C-1 | 97 | C-5 | 3 |
| Re-5 | F-5 | 4 | | | C-1 | 95 | C-4 | 5 |
| Re-6 | F-6 | 2 | | | C-1 | 95 | C-5 | 5 |
| Re-7 | | | | | C-1 | 100 | | |
| Re-8 | | | | | C-1 | 90 | C-3 | 10 |
| Re-9 | F-1 | 2.5 | | | C-2 | 70 | C-3 | 30 |
| Re-10 | F-2 | 4.5 | G-4 | 3 | C-2 | 90 | C-4 | 10 |
| Re-11 | | | G-5 | 3.2 | C-2 | 85 | C-5 | 15 |
| Re-12 | F-6 | 4 | | | C-1 | 97 | C-5 | 3 |
| Re-13 | F-6 | 3.5 | | | C-2 | 97 | C-3 | 3 |
| Re-14 | | | G-2 | 4 | C-2 | 60 | C-3 | 40 |
| Re-15 | | | G-4 | 3.5 | C-1 | 70 | C-3 | 30 |
| Re-16 | F-1 | 2 | G-1 | 4 | C-2 | 90 | C-4 | 10 |
| Re-17 | F-2 | 2 | G-5 | 4 | C-1 | 80 | C-4 | 20 |
| Re-18 | F-4 | 3 | G-3 | 2 | C-1 | 90 | C-4 | 10 |
| Re-19 | F-3 | 2 | | | C-1 | 70 | C-4 | 30 |
| Re-20 | F-6 | 4 | G-3 | 2.8 | C-2 | 90 | C-5 | 10 |
| Re-21 | | | G-2 | 3 | C-2 | 80 | C-5 | 20 |
| Re-22 | | | G-4 | 3 | C-2 | 95 | C-5 | 5 |
| Re-23 | F-5 | 2 | | | C-2 | 90 | C-4 | 10 |
| Re-24 | F-2 | 2 | | | C-2 | 85 | C-4 | 15 |
| Re-25 | F-4 | 2 | | | C-2 | 70 | C-4 | 30 |
| Re-A | F-1 | 2 | G-1 | 4 | C-1 | 80 | C-2 | 20 |
| Re-B | F-3 | 4 | G-4 | 2.8 | C-2 | 80 | C-3 | 20 |
| Re-C | — | | | | C-2 | 80 | C-5 | 5 |
| Re-D | F-4 | 2 | | | C-1 | 95 | C-4 | 10 |
| Re-E | F-5 | 2 | G-5 | 4 | C-1 | 90 | C-3 | 5 |

Details of the abbreviations other than the above-mentioned abbreviations described in Table 6 are shown below

In addition, D-1 to D-13 are the same compounds as D-1 to D-13, respectively, as described above.

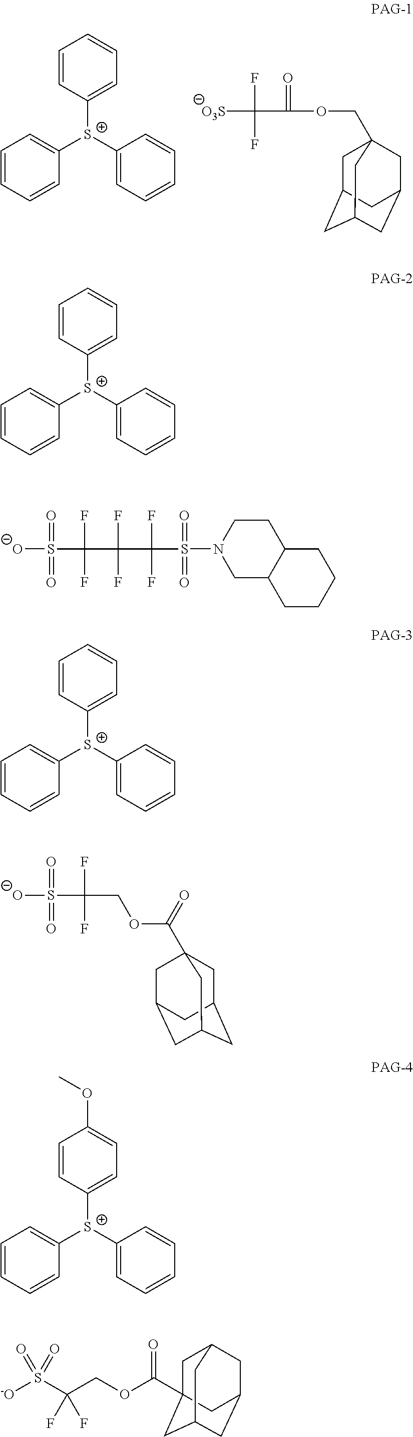

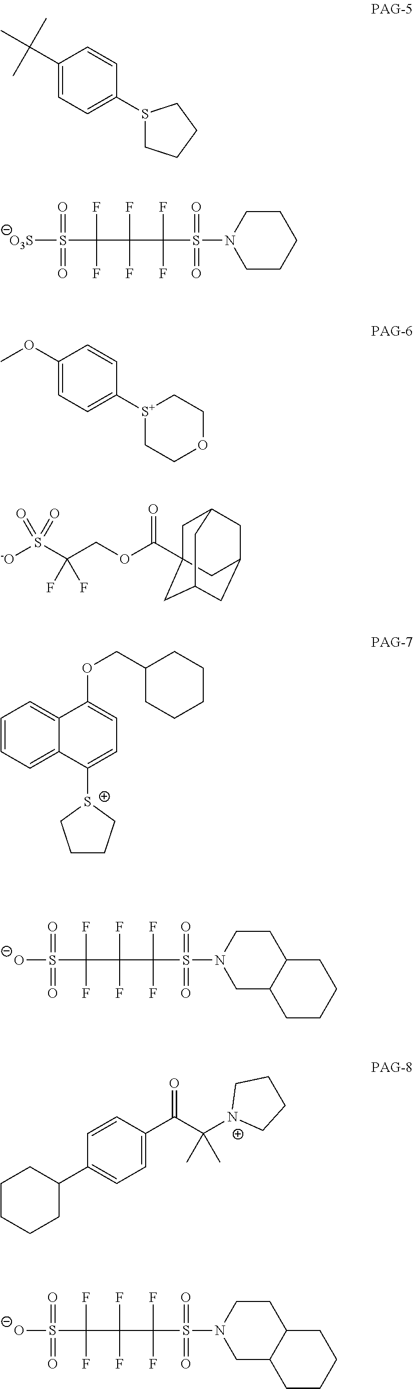

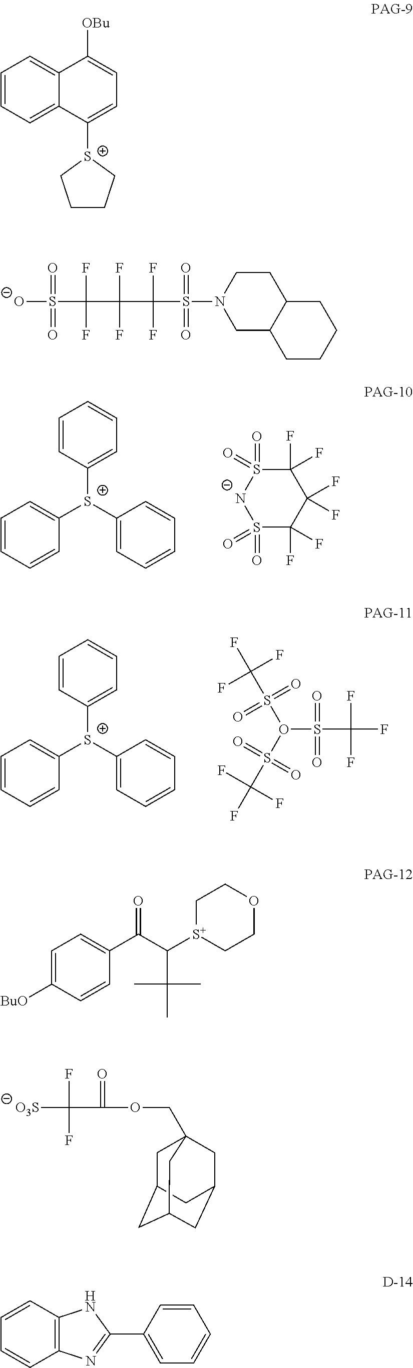

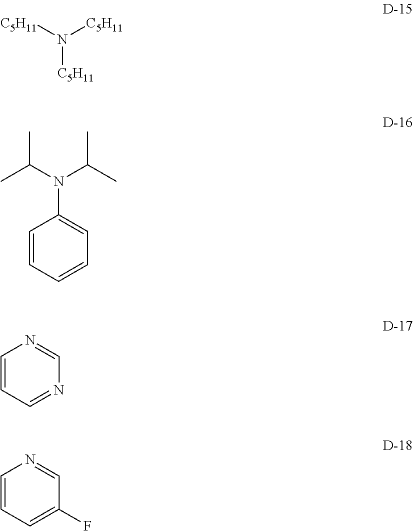

Furthermore, the boiling points of D-1 to D-18 and the pKa of the conjugate acid are shown in Table 7.

TABLE 7

| | pKa | Boiling point (° C.) |
|---|---|---|
| D-1 | 0.45 | >300 |
| D-2 | 0.84 | >300 |
| D-3 | 1.1 | 210 |
| D-4 | 0.8 | 291 |
| D-5 | −0.65 | 188 |
| D-6 | 1.54 | >300 |
| D-7 | 2.71 | 261 |
| D-8 | 0.35 | 252 |
| D-9 | 1.56 | 252 |
| D-10 | 2.21 | >300 |
| D-11 | 1.37 | >300 |
| D-12 | 2.09 | 278 |
| D-13 | 0.12 | >300 |
| D-14 | 5.35 | >300 |
| D-15 | 9.99 | 251 |
| D-16 | 8.05 | 211 |
| D-17 | 1.29 | 126 |
| D-18 | 2.81 | 101 |

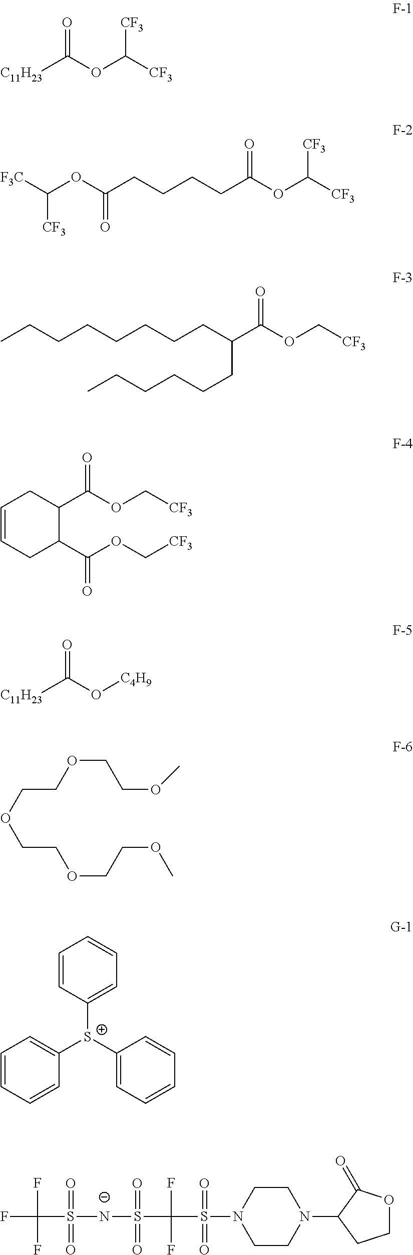

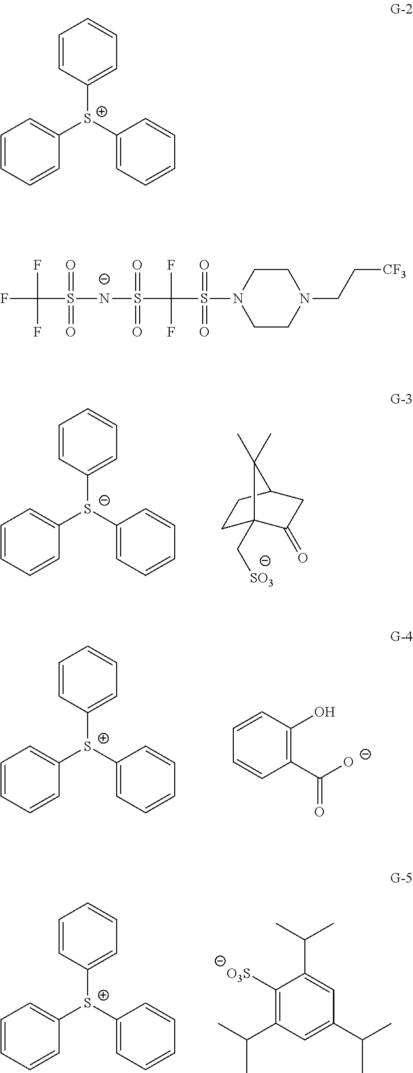

C-1: Propylene glycol monomethyl ether acetate (PGMEA)

C-2: Cyclohexanone

C-3: Propylene glycol monomethyl ether (PGME)

C-4: γ-Butyrolactone

C-5: Propylene carbonate

C-6: 2-Ethylbutnaol

C-7: Perfluorobutyl tetrahvdrofuran

C-8: 4-Methyl-2-pentanol

<Preparation of Topcoat Composition:>

By the same method as for the preparation of the photosensitive resin composition, except that the resin described in Table 8 was dissolved in the solvent described in Table 7 to prepare a solution having a concentration of the solid contents of 3% by mass in Examples in which topcoat compositions were used, a topcoat composition was prepared and a protective film (topcoat) having a film thickness of 50 nm was formed on a resist film which will be described later and evaluated.

TABLE 8

| | Resin | | Additive | | Surfactant | | Solvent | |
|---|---|---|---|---|---|---|---|---|
| Topcoat | | Parts by | | Parts by | | Parts by | | Mixing ratio |
| composition | Type | mass | Type | mass | Type | mass | Type | (mass) |
| TC-1 | PT-1 | 10 | DT-1/DT-2 | 1.3/0.06 | | | FT-1/FT-2 | 70/30 |
| TC-2 | PT-2 | 10 | DT-3/DT-4 | 0.04/0.06 | H-3 | 0.005 | FT-1/FT-3 | 75/25 |
| TC-3 | PT-3 | 10 | DT-5 | 0.05 | | | FT-1/FT-3 | 10/90 |

Details of the abbreviations other than the above-mentioned abbreviations described in Table 8 are shown below.

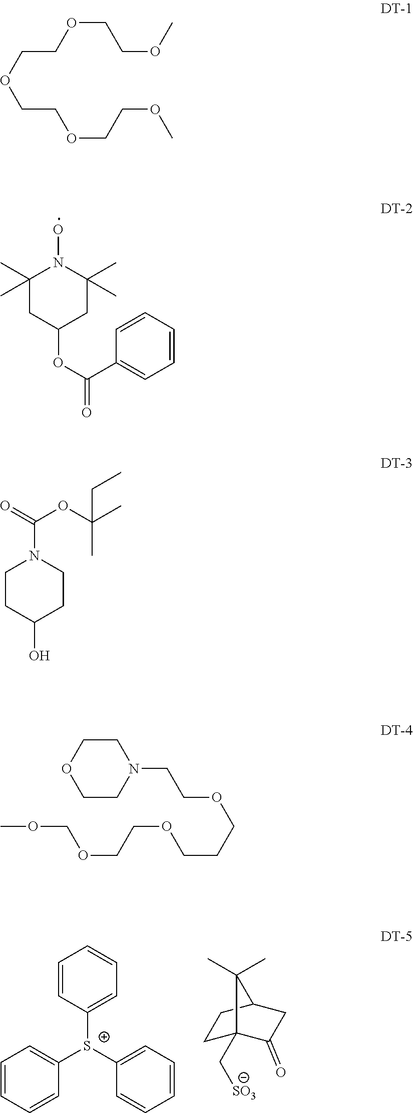

FT-1: 4-Methyl-2-pentanol (MIBC)

FT-2: n-Decane

FT-3: Diisoamyl ether

<Evaluation>

Evaluation of Tolerance of Depth of Focus during Formation of Punched Pattern (Test on DOF Performance)

An antireflection film, ARC29A (manufactured by Nissan Chemical Industries, Ltd.), was applied onto a silicon wafer (diameter: 12 inches) and baked at 205° C. for 60 seconds to form an antireflection film having a film thickness of 86 nm. The photosensitive resin composition prepared above was applied thereon and the film was baked at 100° C. for 60 seconds to form a photosensitive film (resist film) having a film thickness of 100 nm. The obtained wafer was exposed through a 6% half-tone mask with an opening portion in a dimension of 100 nm and a pitch between holes of 800 nm using an ArF excimer laser liquid immersion scanner (ASML manufactured by XT1700i. NA 1.20, Annular, outer sigma 0.700, inner sigma 0.400, XY polarization). Ultrapure water was used as the immersion liquid. Thereafter, the wafer was heated at 100° C. for 60 seconds, developed with an aqueous tetratmethylammonium hydroxide solution (2.38% by mass) for 30 seconds, rinsed with pure water, and then spin-dried to obtain an isolated hole pattern having a diameter of 85 nm.

An exposure dose and a depth of focus for forming an isolated hole pattern having a diameter of 85 nm were defined as an optimal exposure dose and an optimal depth of focus, respectively, and a depth of focus (nm) which allowed a diameter in a range of the diameter ±10% (that is, 85 nm ±10%) in a case where the depth of focus varied (defocus) from the optimal depth of focus was observed. This value was evaluated in accordance with the following standard.

A: The width of the depth of focus is 130 nm or more.

B: The width of the depth of focus is 100 nm or more and less than 130 nm.

C: The width of the depth of focus is less than 100 nm.

Pattern Linearity (Line Width Roughness (LWR))

A line-and-space pattern was obtained under the same condition as above, except that exposure was performed through a 6% half-tone mask of a 1:1 line-and-space pattern having a line width of 75 nm. With regard to a 75 nm (1:1) line-and-space resist pattern resolved with an optimal exposure dose which is an exposure dose for the resolution of a line pattern having a line size of 75 nm on average, in a case where the pattern was observed from the upper part thereof using a critical dimension scanning electron microscope (SEM S-938011 manufactured by Hitachi High Technologies Corporation), the line width was measured at any 32 points and a measurement deviation thereof was evaluated as 3σ (unit: nm). The evaluation standards are shown below. A smaller value of 3σ indicates better performance,

A: The value of 3σ is 5 nm or less.

B: The value of 3σ is more than 5 nm and 7 nm or less.

C: The value of 3σ is more than 7 nm.

The evaluation results are summarized in Table 9.

TABLE 9

| | Photosensitive | Topcoat | | |
|---|---|---|---|---|
| | resin composition | composition | DOF | LWR |
| Example 1 | Re-1 | | A | A |
| Example 2 | Re-2 | | A | B |
| Example 3 | Re-3 | | B | A |
| Example 4 | Re-4 | | A | A |
| Example 5 | Re-5 | | A | A |
| Example 6 | Re-6 | | B | A |
| Example 7 | Re-7 | | A | B |
| Example 8 | Re-8 | | B | A |
| Example 9 | Re-9 | | A | A |
| Example 10 | Re-10 | | A | B |
| Example 11 | Re-11 | | A | A |
| Example 12 | Re-12 | | A | A |
| Example 13 | Re-13 | | B | A |
| Example 14 | Re-14 | TC-2 | A | A |
| Example 15 | Re-15 | TC-3 | B | B |
| Example 16 | Re-16 | | A | A |
| Example 17 | Re-17 | | B | A |
| Example 18 | Re-18 | | A | A |
| Example 19 | Re-19 | | A | A |
| Example 20 | Re-20 | | B | A |
| Example 21 | Re-21 | | A | A |
| Example 22 | Re-22 | | A | A |
| Example 23 | Re-23 | | B | B |
| Example 24 | Re-24 | | B | A |
| Example 25 | Re-25 | | A | A |
| Comparative Example 1 | Re-A | | C | C |
| Comparative Example 2 | Re-B | | C | C |
| Comparative Example 3 | Re-C | | C | C |
| Comparative Example 4 | Re-D | | C | C |
| Comparative Example 5 | Re-E | | C | C |

As shown in Table 9, the photosensitive resin composition according to the present disclosure has a high tolerance of a depth of focus during formation of a punched pattern, as compared with the photosensitive resin compositions of Comparative Examples.

In addition, a pattern having excellent linearity was obtained with the photosensitive resin composition according to the present disclosure.

What is claimed is:

1. A photosensitive resin composition comprising:
a resin having a constitutional unit having an acid-decomposable group;
a photoacid generator;
a solvent; and
a compound represented by Formula D, 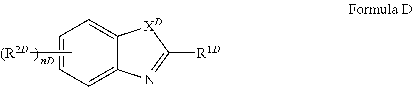
in Formula D, X^{D} represents an O atom or an S atom, R^{1D} represents a hydrogen atom, a hydrocarbon group, an acyl group, an acyloxy group, or an alkoxycarbonyl group, R^{2D} represents a halogen atom, an alkyl group, an alkenyl group, an aryl group, a hydroxyl group, an alkoxy group, an aryloxy group, a mercapto group, an alkylthio group, an arylthio group, an acyl group, an acyloxy group, an alkoxycarbonyl group, a cyano group, or a nitro group, nD represents an integer from 0 to 4, and two or more of R^{2D}'s may be bonded to each other to form a ring.

2. The photosensitive resin composition according to claim 1,
wherein a pKa of a conjugate acid of the compound represented by Formula D is 0.0 to 3.0.

3. The photosensitive resin composition according to claim 1,
wherein the compound represented by Formula D is a compound represented by Formula D-1, 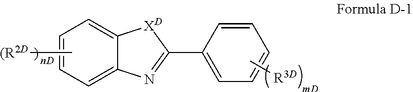
in Formula D-1, X^{D} represents an O atom or an S atom, R^{2D} and R^{3D} each independently represent a halogen atom, an alkyl group, an alkenyl group, an aryl group, a hydroxyl group, an alkoxy group, an aryloxy group, a mercapto group, an alkylthio group, an arylthio group, an acyl group, an acyloxy group, an alkoxycarbonyl group, a cyano group, or a nitro group, nD represents an integer from 0 to 4, mD represents an integer from 0 to 5, and two or more of R^{2D}'s may be bonded to each other to form a ring.

4. The photosensitive resin composition according to claim 1,
wherein the compound represented by Formula. D is a compound represented by Formula D-2, 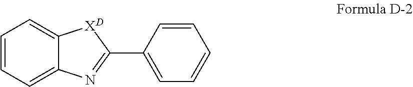
in Formula D-2, X^{D} represents an O atom or an S atom.

5. The photosensitive resin composition according to claim 1,
wherein the photoacid generator is an ionic compound including a cation and an anion, and the anion includes an ion represented by any one of Formula An-1, Formula An-2, or Formula An-3, 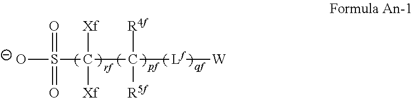
in Formula An-1, pf represents an integer of 0 to 10, qf represents an integer of 0 to 10, rf represents an integer of 1 to 3, Xf's each independently represent a fluorine atom or an alkyl group substituted with at least one fluorine atom, in a case where rf is an integer of 2 or more, a plurality of —C(XF)₂-'s may be the same as or different from each other, R^{4f} and R^{5f} each independently represent a hydrogen atom, a fluorine atom, an alkyl group, or an alkyl group substituted with at least one fluorine atom, in a case where pf is an integer of 2 or more, a plurality of —CR^{4f}R^{5f}-'s may be the same as or different from each other, L^{f} represents a divalent linking group, in a case where qf is an integer of 2 or more, a plurality of L^{f}-'s may be the same as or different from each other, and W represents an organic group including a cyclic structure, 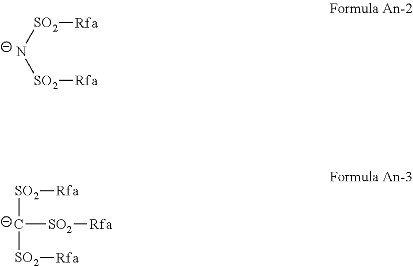
in Formula An-2 and Formula An-3, Rfa's each independently represent a monovalent organic group having a fluorine atom, and a plurality of Rfa's may be bonded to each other to form a ring.

6. The photosensitive resin composition according to claim 1,
wherein the resin having a constitutional unit having an acid-decomposable group includes a constitutional unit represented by Formula III, 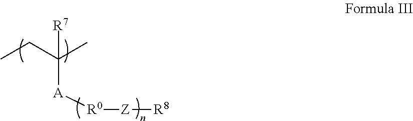
in Formula III, A represents an ester bond or an amide bond, n represents an integer of 0 to 5, R⁰ represents an alkylene group, a cycloalkylene group, or a combination thereof, Z represents a single bond, an ether bond, an ester bond, an amide bond, a urethane bond, or a urea bond, R⁸ represents a monovalent organic group having a lactone structure or a sultone structure, and R⁷ represents a hydrogen atom, a halogen atom, or a monovalent organic group.

7. The photosensitive resin composition according to claim 1,
wherein the constitutional unit having an acid-decomposable group includes a constitutional unit represented by Formula AI, 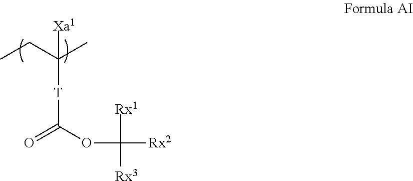
in Formula AI, Xa¹ represents a hydrogen atom, a halogen atom, or a monovalent organic group. T represents a single bond or a divalent linking group, Rx¹ to Rx³ each independently represent an alkyl group or a cycloalkyl group, and any two of Rx¹ to Rx³ may or may not be bonded to each other to form a ring structure,

8. The photosensitive resin composition according to claim 1, further comprising a fluorine-containing resin having a constitutional unit represented by Formula X, 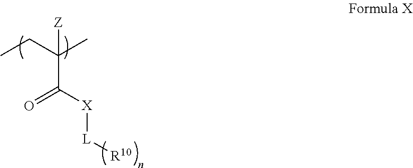
in Formula X, Z represents a halogen atom, a group represented by R¹¹OCH₂—, or a group represented by R¹²OC(═O)CH₂—, R¹¹ and R¹² each independently represent a substituent, X represents an oxygen atom or a sulfur atom, L represents an n+1-valent linking group, R¹⁰ represents a group having a group that decomposes by the action of an aqueous alkali solution to increase a solubility of the fluorine-containing resin in an aqueous alkali solution, n represents a positive integer, and in a case where n is 2 or more, a plurality of R¹⁰'s may be the same as or different from each other.

9. The photosensitive resin composition according to claim 1, further comprising a compound represented by Formula B, 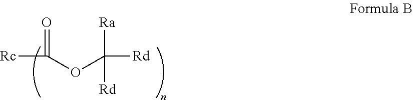
in Formula B, Ra represents an electron-withdrawing group, Rc represents an n-valent hydrocarbon group, Rd's each independently represent a hydrogen atom or a substituent, and n represents an integer of 1 to 3.

10. The photosensitive resin composition according to claim 1, further comprising a compound represented by any one of Formula d1-1, Formula d1-2, or Formula d1-3, 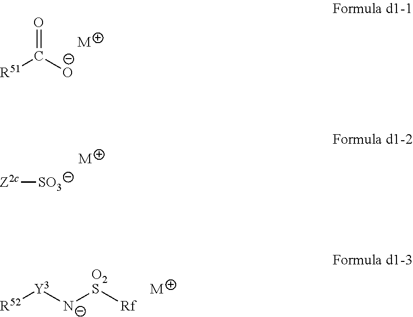
in Formula d1-1 to Formula d1-3, R⁵¹ represents a hydrocarbon group which may have a substituent, Z^{2c} represents a hydrocarbon group having 1 to 30 carbon atoms, which may have a substituent, in which a fluorine atom is not bonded to the carbon atom adjacent to the S atom, R⁵² represents an organic group, Y³ represents a linear, branched, or cyclic alkylene group or an arylene group, Rf represents a hydrocarbon group including a fluorine atom, and M⁺'s each independently represent an ammonium cation, a sulfonium cation, or an iodonium cation.

11. The photosensitive resin composition according to claim 1,
wherein the solvent includes γ-hutyrolactone.

12. The photosensitive resin composition according to claim 1, further comprising a low-molecular-weight ester compound having alkali decomposability and having a molecular weight of less than 1,500.

13. A resist film which is a solidified product of the photosensitive resin composition according to claim 1.

14. A pattern forming method comprising:
exposing the resist film according to claim 13; and
developing the exposed resist film.

15. A method for manufacturing an electronic device, comprising the pattern forming method according to claim 4.
